SEQUENCE LISTING (119 sequences)

SEQ ID NO: 1 - DNA, 24 bp, Artificial Sequence
  Oligonucleotide primer
cagaaacacctgtaaggaccagaa

SEQ ID NO: 2 - DNA, 24 bp, Artificial Sequence
  Oligonucleotide primer
gcaggagctagagagggataagaa

SEQ ID NO: 3 - DNA, 25 bp, Artificial Sequence
  Oligonucleotide primer
ggccaagaatgtggtgaaagtaaat

SEQ ID NO: 4 - DNA, 21 bp, Artificial Sequence
  Oligonucleotide primer
tgaagacacgtgggtcgaatg

SEQ ID NO: 5 - DNA, 25 bp, Artificial Sequence
  Oligonucleotide primer
gctgaactagcaactaagaaaccac

SEQ ID NO: 6 - DNA, 20 bp, Artificial Sequence
  Oligonucleotide primer
cagcctgcgagacctaagag

SEQ ID NO: 7 - DNA, 24 bp, Artificial Sequence
  Oligonucleotide primer
gggacacgtaaatggtcttcagtt

SEQ ID NO: 8 - DNA, 19 bp, Artificial Sequence
  Oligonucleotide primer
cgcactgcgaggagaagat

SEQ ID NO: 9 - DNA, 25 bp, Artificial Sequence
  Oligonucleotide primer
actgcagtggaaaaacatcttgact

SEQ ID NO: 10 - DNA, 25 bp, Artificial Sequence
  Oligonucleotide primer
gcaagtcatcttccctacccatatt

SEQ ID NO: 11 - DNA, 17 bp, Artificial Sequence
  Oligonucleotide primer
ggcctttgcgcgtacag

SEQ ID NO: 12 - DNA, 18 bp, Artificial Sequence
  Oligonucleotide primer
gccgaccaaagggaagca

SEQ ID NO: 13 - DNA, 27 bp, Artificial Sequence
  Oligonucleotide primer
caagcatgttgtgaagttgaaagatga

SEQ ID NO: 14 - DNA, 24 bp, Artificial Sequence
  Oligonucleotide primer
gctggcagagttattggaaaagga

SEQ ID NO: 15 - DNA, 25 bp, Artificial Sequence
  Oligonucleotide primer
gaaactgaatgaccaagcaaacact

SEQ ID NO: 16 - DNA, 22 bp, Artificial Sequence
  Oligonucleotide primer
aggaggaaacggtgaaggacta

SEQ ID NO: 17 - DNA, 25 bp, Artificial Sequence
  Oligonucleotide primer
ccgatttgtggaggatgagaaagat

SEQ ID NO: 18 - DNA, 20 bp, Artificial Sequence
  Oligonucleotide primer
ccctgcaactggaagaagga

SEQ ID NO: 19 - DNA, 20 bp, Artificial Sequence
  Oligonucleotide primer
aggaagggttctgccagaga

SEQ ID NO: 20 - DNA, 22 bp, Artificial Sequence
  Oligonucleotide primer
gcattcagcctagttcctggtt

SEQ ID NO: 21 - DNA, 20 bp, Artificial Sequence
  Oligonucleotide primer
agcgagctctcaaagcaaga

SEQ ID NO: 22 - DNA, 18 bp, Artificial Sequence
  Oligonucleotide primer
cctgcaggcatccctgta

SEQ ID NO: 23 - DNA, 20 bp, Artificial Sequence
  Oligonucleotide primer
ccgtccctctctgacagttc

SEQ ID NO: 24 - DNA, 25 bp, Artificial Sequence
  Oligonucleotide primer
cctcagatgatgcctatccagaaat

SEQ ID NO: 25 - DNA, 21 bp, Artificial Sequence
  Oligonucleotide primer
cgtgggtgtcatgatgatgca

SEQ ID NO: 26 - DNA, 21 bp, Artificial Sequence
  Oligonucleotide primer
caaatggctgccaaactggaa

SEQ ID NO: 27 - DNA, 22 bp, Artificial Sequence
  Oligonucleotide primer
cgccttccagctgttacatctt

SEQ ID NO: 28 - DNA, 25 bp, Artificial Sequence
  Oligonucleotide primer
tggctcatccctatgttcaaattca

SEQ ID NO: 29 - DNA, 25 bp, Artificial Sequence
  Oligonucleotide primer
gctatcctctactgcttcctcaatg

SEQ ID NO: 30 - DNA, 20 bp, Artificial Sequence
  Oligonucleotide primer
cccgtgtcaacgagataagc

SEQ ID NO: 31 - DNA, 20 bp, Artificial Sequence
  Oligonucleotide primer
agcttcccacagcatgaaga

SEQ ID NO: 32 - DNA, 20 bp, Artificial Sequence
  Oligonucleotide primer
gagtcctccaaaccaacagc

SEQ ID NO: 33 - DNA, 21 bp, Artificial Sequence
  Oligonucleotide primer
agaggagcgagatgttcaaga

SEQ ID NO: 34 - DNA, 20 bp, Artificial Sequence
  Oligonucleotide primer
cctcggtgaggtagaccact

SEQ ID NO: 35 - DNA, 24 bp, Artificial Sequence
  Oligonucleotide primer
tttagaactcagtagccatcttgc

SEQ ID NO: 36 - DNA, 22 bp, Artificial Sequence
  Oligonucleotide primer
tcatgatctgcttgactgtgag

SEQ ID NO: 37 - DNA, 18 bp, Artificial Sequence
  Oligonucleotide primer
atgctggatgccgctact

SEQ ID NO: 38 - DNA, 25 bp, Artificial Sequence
  Oligonucleotide primer
aggaaagatacctcctactccattc

SEQ ID NO: 39 - DNA, 22 bp, Artificial Sequence
  Oligonucleotide primer
ttgtggaaagaagacttggcta

SEQ ID NO: 40 - DNA, 27 bp, Artificial Sequence
  Oligonucleotide primer
tgatagatccattcctatgactgtaga

SEQ ID NO: 41 - DNA, 25 bp, Artificial Sequence
  Oligonucleotide primer
tccatcaccatttgaatagcttgca

SEQ ID NO: 42 - DNA, 22 bp, Artificial Sequence
  Oligonucleotide primer
ttgtaacgggagaggagacctt

SEQ ID NO: 43 - DNA, 22 bp, Artificial Sequence
  Oligonucleotide primer
caggagtttgctgcttgcatac

SEQ ID NO: 44 - DNA, 19 bp, Artificial Sequence
  Oligonucleotide primer
gcccagagcagaggttgtc

SEQ ID NO: 45 - DNA, 24 bp, Artificial Sequence
  Oligonucleotide primer
cttctggccacacttcattattgg

SEQ ID NO: 46 - DNA, 25 bp, Artificial Sequence
  Oligonucleotide primer
cagctaatttgtcccgaaactcatg

SEQ ID NO: 47 - DNA, 24 bp, Artificial Sequence
  Oligonucleotide primer
cagctccacaaagtctcctattcc

SEQ ID NO: 48 - DNA, 19 bp, Artificial Sequence
  Oligonucleotide primer
gctcctgacctcggtacct

SEQ ID NO: 49 - DNA, 23 bp, Artificial Sequence
  Oligonucleotide primer
tggcaaaggagcaaatagtccat

SEQ ID NO: 50 - DNA, 25 bp, Artificial Sequence
  Oligonucleotide primer
catgcctcagatgtttgaaaacctt

SEQ ID NO: 51 - DNA, 22 bp, Artificial Sequence
  Oligonucleotide primer
gcccattcagcaactctttctc

SEQ ID NO: 52 - DNA, 22 bp, Artificial Sequence
  Oligonucleotide primer
tggtttccttcctggagttgtg

SEQ ID NO: 53 - DNA, 20 bp, Artificial Sequence
  Oligonucleotide primer
caagctgacagcggagtttt

SEQ ID NO: 54 - DNA, 25 bp, Artificial Sequence
  Oligonucleotide primer
gacaacaacttctgcacttgacaaa

SEQ ID NO: 55 - DNA, 27 bp, Artificial Sequence
  Oligonucleotide primer
gtcttcactcctttcgtttaagtcaga

SEQ ID NO: 56 - DNA, 21 bp, Artificial Sequence
  Oligonucleotide primer
ccagaacttctccagcccata

SEQ ID NO: 57 - DNA, 18 bp, Artificial Sequence
  Oligonucleotide primer
gtggagtggctcgcctta

SEQ ID NO: 58 - DNA, 19 bp, Artificial Sequence
  Oligonucleotide primer
cgcaccccagttctcaaac

SEQ ID NO: 59 - DNA, 30 bp, Artificial Sequence
  Oligonucleotide primer
tctggattcactaatctagttgtagtcaca

SEQ ID NO: 60 - DNA, 30 bp, Artificial Sequence
  Oligonucleotide primer
gccaattctttcaaaatatgcttcaatgtc

SEQ ID NO: 61 - DNA, 24 bp, Artificial Sequence
  Oligonucleotide primer
ctagtctctcacgaacacaaagct

SEQ ID NO: 62 - DNA, 16 bp, Artificial Sequence
  Oligonucleotide primer
cgggccatcccttggt

SEQ ID NO: 63 - DNA, 25 bp, Artificial Sequence
  Oligonucleotide primer
gtagcatcagatttggaagcctttg

SEQ ID NO: 64 - DNA, 23 bp, Artificial Sequence
  Oligonucleotide primer
ctcttcaggcaggtcaaaactct

SEQ ID NO: 65 - DNA, 23 bp, Artificial Sequence
  Oligonucleotide primer
tccttctcgtcgtcgtagtagtt

SEQ ID NO: 66 - DNA, 24 bp, Artificial Sequence
  Oligonucleotide primer
gttcttccgcacttcttcaatgtg

SEQ ID NO: 67 - DNA, 24 bp, Artificial Sequence
  Oligonucleotide primer
cctgcatggattagcacatagtct

SEQ ID NO: 68 - DNA, 18 bp, Artificial Sequence
  Oligonucleotide primer
ccagttaaccaaatggcc

SEQ ID NO: 69 - DNA, 15 bp, Artificial Sequence
  Oligonucleotide primer
cagcgccgccacttc

SEQ ID NO: 70 - DNA, 18 bp, Artificial Sequence
  Oligonucleotide primer
cagagccatgcggatgta

SEQ ID NO: 71 - DNA, 19 bp, Artificial Sequence
  Oligonucleotide primer
gtggtaaggtccccgtgag

SEQ ID NO: 72 - DNA, 19 bp, Artificial Sequence
  Oligonucleotide primer
tccgctgagcaactttgac

SEQ ID NO: 73 - DNA, 24 bp, Artificial Sequence
  Oligonucleotide primer
tcaggcttcattatgttcttctca

SEQ ID NO: 74 - DNA, 19 bp, Artificial Sequence
  Oligonucleotide primer
ctgtggaggctcagggaat

SEQ ID NO: 75 - DNA, 24 bp, Artificial Sequence
  Oligonucleotide primer
agctctcagacatgtcctatcttt

SEQ ID NO: 76 - DNA, 21 bp, Artificial Sequence
  Oligonucleotide primer
tttttccaacgaatcacctgt

SEQ ID NO: 77 - DNA, 19 bp, Artificial Sequence
  Oligonucleotide primer
cccttagggctgttctgga

SEQ ID NO: 78 - DNA, 18 bp, Artificial Sequence
  Oligonucleotide primer
ataccagggcgaggagga

SEQ ID NO: 79 - DNA, 22 bp, Artificial Sequence
  Oligonucleotide primer
catcttgtttttccttggcttc

SEQ ID NO: 80 - DNA, 27 bp, Artificial Sequence
  Oligonucleotide primer
aagacattctttccagttaaagttgag

SEQ ID NO: 81 - DNA, 21 bp, Artificial Sequence
  Oligonucleotide probe
ctggcttaagtcttgaaacta

SEQ ID NO: 82 - DNA, 17 bp, Artificial Sequence
  Oligonucleotide probe
tcatgctggccaccttg

SEQ ID NO: 83 - DNA, 18 bp, Artificial Sequence
  Oligonucleotide probe
cttgatggcgatgaattt

SEQ ID NO: 84 - DNA, 19 bp, Artificial Sequence
  Oligonucleotide probe
caagtcaaagggatcttca

SEQ ID NO: 85 - DNA, 16 bp, Artificial Sequence
  Oligonucleotide probe
caaagctctgaaaatc

SEQ ID NO: 86 - DNA, 19 bp, Artificial Sequence
  Oligonucleotide probe
cagaccatcaagcaataca

SEQ ID NO: 87 - DNA, 17 bp, Artificial Sequence
  Oligonucleotide probe
ctgctgaggatttcttt

SEQ ID NO: 88 - DNA, 18 bp, Artificial Sequence
  Oligonucleotide probe
ttggtggtgatgataacc

SEQ ID NO: 89 - DNA, 22 bp, Artificial Sequence
  Oligonucleotide probe
tccaggattttcaatatgtccc

SEQ ID NO: 90 - DNA, 25 bp, Artificial Sequence
  Oligonucleotide probe
cttgaggaaagaaatctagtattat

SEQ ID NO: 91 - DNA, 17 bp, Artificial Sequence
  Oligonucleotide probe
ctgctcaatggactttc

SEQ ID NO: 92 - DNA, 15 bp, Artificial Sequence
  Oligonucleotide probe
aagacccggaaaacc

SEQ ID NO: 93 - DNA, 20 bp, Artificial Sequence
  Oligonucleotide probe
caactcaaatcggaagtatc

SEQ ID NO: 94 - DNA, 18 bp, Artificial Sequence
  Oligonucleotide probe
ttcattcaccgttttgcc

SEQ ID NO: 95 - DNA, 18 bp, Artificial Sequence
  Oligonucleotide probe
caaagacccagaacatca

SEQ ID NO: 96 - DNA, 16 bp, Artificial Sequence
  Oligonucleotide probe
cagttggccagcttca

SEQ ID NO: 97 - DNA, 19 bp, Artificial Sequence
  Oligonucleotide probe
aatgagacgagaacacttc

SEQ ID NO: 98 - DNA, 18 bp, Artificial Sequence
  Oligonucleotide probe
tttggagccgactgcaag

SEQ ID NO: 99 - DNA, 20 bp, Artificial Sequence
  Oligonucleotide probe
ccagaagactaaagcttcac

SEQ ID NO: 100 - DNA, 16 bp, Artificial Sequence
  Oligonucleotide probe
ctggccaaagggatca

SEQ ID NO: 101 - DNA, 18 bp, Artificial Sequence
  Oligonucleotide probe
attggagcagaaagaaca

SEQ ID NO: 102 - DNA, 16 bp, Artificial Sequence
  Oligonucleotide probe
ccccatccagtggctg

SEQ ID NO: 103 - DNA, 16 bp, Artificial Sequence
  Oligonucleotide probe
cttcagcgagaacttt

SEQ ID NO: 104 - DNA, 20 bp, Artificial Sequence
  Oligonucleotide probe
cttcaatcctctagactttg

SEQ ID NO: 105 - DNA, 16 bp, Artificial Sequence
  Oligonucleotide probe
ccggacaggcctctac

SEQ ID NO: 106 - DNA, 19 bp, Artificial Sequence
  Oligonucleotide probe
tttgcgagagaaggataag

SEQ ID NO: 107 - DNA, 19 bp, Artificial Sequence
  Oligonucleotide probe
cagcctgaacatttcccac

SEQ ID NO: 108 - DNA, 15 bp, Artificial Sequence
  Oligonucleotide probe
ccgcctgcacctcac

SEQ ID NO: 109 - DNA, 8 bp, Artificial Sequence
  Oligonucleotide probe
ccaggctg

SEQ ID NO: 110 - DNA, 8 bp, Artificial Sequence
  Oligonucleotide probe
cttcctcc

SEQ ID NO: 111 - DNA, 8 bp, Artificial Sequence
  Oligonucleotide probe
ggcagcag

SEQ ID NO: 112 - DNA, 8 bp, Artificial Sequence
  Oligonucleotide probe
gaaggcag

SEQ ID NO: 113 - DNA, 8 bp, Artificial Sequence
  Oligonucleotide probe
ctgggcaa

SEQ ID NO: 114 - DNA, 8 bp, Artificial Sequence
  Oligonucleotide probe
tctggagc

SEQ ID NO: 115 - DNA, 8 bp, Artificial Sequence
  Oligonucleotide probe
caggagaa

SEQ ID NO: 116 - DNA, 8 bp, Artificial Sequence
  Oligonucleotide probe
ctggggct

SEQ ID NO: 117 - DNA, 8 bp, Artificial Sequence
  Oligonucleotide probe
ctcctcct

SEQ ID NO: 118 - DNA, 8 bp, Artificial Sequence
  Oligonucleotide probe
ggaggctg

SEQ ID NO: 119 - DNA, 8 bp, Artificial Sequence
  Oligonucleotide probe
tggtggag

(19) United States
(12) Patent Application Publication
(10) Pub. No.: US 2011/0159498 A1
(43) Pub. Date: Jun. 30, 2011

(54) METHODS, AGENTS AND KITS FOR THE DETECTION OF CANCER

(75) Inventors: Kuo-Jang Kao, Gainesville, FL (US); Ta-Yuan Chen, Taiwan (CN); To-Yu Huang, Taiwan (CN); Andrew T. Huang, Durham, NC (US)

(73) Assignee: China Synthetic Rubber Corporation, Taipei , TAIWAN (CN)

(21) Appl. No.: 12/937,207

(22) PCT Filed: Apr. 8, 2009
(86) PCT No.: PCT/US09/02196

§ 371 Date: Nov. 22, 2010

Related U.S. Application Data
(60) Provisional application No. 61/123,761, filed on Apr. 11, 2008.

(51) Int. Cl. C12Q 1/68

(52) U.S. Cl. 435/6.12

(57) ABSTRACT

The present invention relates to methods of diagnosing a cancer in a subject, and methods of providing a prognosis for a subject that has a cancer. The invention also relates to diagnostic and prognostic kits for cancer.

### RELATED APPLICATION

This application claims the benefit of U.S. Provisional Application No. 61/123,761, filed on Apr. 11, 2008. The entire teachings of the above application are incorporated herein by reference.

### BACKGROUND OF THE INVENTION

Cancer, a group of diseases characterized by uncontrolled growth and spread of malignant cells, is a significant cause of human mortality and morbidity world-wide, and a national economic burden in the United States.

Like all living cells, the behavior of cancer cells is controlled by the expression of a large number of different genes. Genes that are differentially expressed between cancer cells and normal cells, or between two different types of cancer cells, collectively constitute a gene expression profile that can be used to detect the presence of a cancer in an individual, classify tumor subtypes and/or predict a patient's clinical outcome. In addition, the products of these genes (e.g., mRNA, protein) provide potential targets for therapy.

The successful treatment of cancer depends, in part, on early detection and diagnosis of the cancer in an individual. Accordingly, there is a need for the identification of gene expression profiles that can be relied upon for the accurate detection and diagnosis of various types of cancers at early stages. In addition, there is a further need for a gene expression profile that includes genes that are common to many different types of cancers and, thus, can be used to screen a large patient population for the presence of a cancer. There is also a need for more efficient methods of identifying useful gene expression profiles for cancer.

### SUMMARY OF THE INVENTION

The present invention encompasses, in one embodiment, a method of diagnosing whether a subject has a cancer. The method comprises detecting in a sample from the subject the level of expression of a subset of genes that are overexpressed in the cancer. According to the invention, the genes in the subset are selected from the group of genes known in the art as MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARP1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1 and NUSAP1. Increased levels of expression of the subset of genes in the sample from the subject, relative to a control, indicate that the subject has a cancer.

In another embodiment, the invention relates to a method of providing a prognosis for a subject that has a cancer, comprising detecting the level of expression of one or more genes selected from the group consisting of PRC1, CENPF, RDBP, CCNB2 and RAD54B in a sample from the subject, and comparing the level of expression of the gene in the sample to a control. An increased level of expression of PRC1, CENPF, RDBP, CCNB2 and/or RAD54B in the sample from the subject, relative to the control, indicates a poor prognosis (e.g., an increased risk of metastasis). In a particular embodiment, the cancer is hepatocellular carcinoma, nasopharyngeal cancer or breast cancer.

In a further embodiment, the invention relates to a method of providing a prognosis for a subject that has a cancer, comprising detecting the level of expression of one or more genes selected from the group consisting of CDC2, CCHCR1, and HMGA1 in a sample from the subject, and comparing the level of expression of that gene in the sample to a control. An increased level of expression of CDC2, CCHCR1, and/or HMGA1 in the sample from the subject, relative to the control, indicates a poor prognosis (e.g., shorter survival). In a particular embodiment, the cancer is hepatocellular carcinoma, nasopharyngeal cancer or breast cancer.

The present invention also provides, in one embodiment, a kit for diagnosing whether a subject has a cancer, comprising a collection of probes capable of detecting the level of expression of at least about twenty genes selected from the group consisting of the genes known in the art as MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARP1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1 and NUSAP1. In a particular embodiment, the probes are nucleic acid probes that hybridize to RNA (e.g., mRNA) products of these genes. In another embodiment, the probes are antibodies that bind to proteins encoded by these genes.

The invention also provides, in another embodiment, a kit for determining a prognosis (e.g., risk of metastasis) for a subject that has a cancer, comprising a probe that is capable of detecting the level of expression of one or more genes selected from the group consisting of PRC1, CENPF, RDBP, CCNB2 and RAD54B.

In yet another embodiment, the invention further provides a kit for determining a prognosis (e.g., survival) for a subject that has a cancer, comprising a probe that is capable of detecting the level of expression of one or more genes selected from the group consisting of PRC1, CDC2, CCHCR1, and HMGA1.

In another embodiment, the invention relates to a method of determining a gene expression profile for a cancer. The method comprises detecting the expression of genes in both cancerous and non-cancerous samples from the same individual (i.e., subject) and identifying genes that are differentially expressed between the cancerous and non-cancerous samples. According to the method, a gene that is differentially expressed between the cancerous sample and the non-cancerous sample is included in a gene expression profile for the cancer.

In an additional embodiment, the invention relates to a method of diagnosing whether a subject has a cancer. The method comprises detecting in a sample from the subject the level of expression of a subset of genes that are underexpressed in the cancer. According to the invention, the genes in the subset are selected from the group of genes known in the art as NAT2, CD5L, CXCL14, VIPR1, CCL14/15, FCN3, CRHBP, GPD1, KCNN2, HGFAC, FOSB, LCAT, MARCO, CYP1A2, FCN2, and DPT. Decreased levels of expression, or an absence of expression, of the subset of genes in the sample from the subject, relative to a control, indicate that the subject has a cancer.

In a further embodiment, the invention provides a kit for diagnosing whether a subject has a cancer, comprising a collection of probes capable of detecting the level of expression of at least about five genes selected from the group consisting of the genes known in the art as NAT2, CD5L, CXCL14, VIPR1, CCL14/15, FCN3, CRHBP, GPD1, KCNN2, HGFAC, FOSB, LCAT, MARCO, CYP1A2, FCN2, and DPT. In a particular embodiment, the probes are nucleic acid probes that hybridize to RNA (e.g., mRNA) products of these genes. In another embodiment, the probes are antibodies that bind to proteins encoded by these genes.

The diagnostic and prognostic methods and the kits for cancer that are provided by the present invention are based, in part, on the discovery of a universal gene expression profile, or common neoplastic signature, that is capable of distinguishing tissue samples of many different types and subtypes of cancer from corresponding normal tissue samples, and predicting clinical survival outcomes for multiple types of cancers. Unlike many gene expression profiles for cancer that have been reported previously (Whitfield M L, et al. Nature Review Cancer 6:99-106 (2006); Rhodes D R, et al. Proc. Nat. Acad. Sci. USA 101:9309-9314 (2004); see FIG. 33), which were determined by assembling information from various reports in the literature, and are frequently based on a single cancer and/or are limited to a particular feature of a cancer (e.g., proliferation, neoplastic transformation), the common neoplastic signature described herein has been determined experimentally, and has been shown to be universal for cancer using a systematic study.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a flow chart diagram depicting an algorithm for the identification of genes that show significant differential expression between tumor and adjacent non-tumorous tissues.

FIG. 2 is a graph depicting an example of the density distribution of probe-sets on an array showing significant expression differences (p<0.05) between tumor and normal tissue when 41 probe-sets are randomly selected. Random selection was repeated 10,000 times. Values along the y-axis indicate the density of genes with a p-value less than 0.05.

FIG. 3 is a chart showing p-values for the number of probe sets (second row, entitled “Number of selected probe sets”) selected at different stringencies (first row, entitled “Stringency of probe selection”) that differentiate cancer from corresponding normal tissues for each of the listed cancers (left column). The total number of different cancers showing a p-value of less than 0.005 are listed in the bottom row. A selection stringency of 12 differentiated the greatest number of cancers from corresponding normal tissues (19 out of 20 different types of cancer). The p values were calculated using a binomial test and indicate how the selected probe sets are enriched to differentiate tumor and corresponding normal tissues compared to randomly selected probe sets.

FIG. 4 is a list of hepatocellular carcinoma (HCC) tumor-specific genes showing significant differential expression in at least 12 of 18 paired HCC and adjacent non-cancerous liver tissue samples (stringency level of 12). The listed genes show significant expression in HCC tissue samples, but not in adjacent non-cancerous liver tissue samples. For each gene, the affymetrix ID number of the corresponding probe-set on the Affymetrix chip (AFFY_ID), the gene symbol, the known or putative function of the gene, and the stringency level at which the gene(s) were selected are shown. A total of 55 genes are represented by the 59 probe-sets, as TOP2A, CCHCR1, HMMR and CDC2 are each represented by two probe-sets. Broad classes of gene functions are assigned a shade as indicated.

FIG. 5 is a list of genes specific for non-cancerous liver tissue, which show significant differential expression in at least 12 of 18 paired HCC and adjacent non-cancerous liver tissue samples. The listed genes show significant expression in non-cancerous liver tissue samples, but not in adjacent HCC tissue samples. For each gene, the affymetrix ID number of the corresponding probe-set on the Affymetrix chip (AFFY_ID), the gene symbol, the function of the gene and the number of 18 paired HCC and adjacent non-cancerous liver tissue samples showing differential expression of the gene at a stringency level of greater than or equal to 12 (Stringency for Selection) are shown. Broad classes of gene functions are assigned a shade as indicated.

FIGS. 6-10 are a series of graphs depicting the expression intensities of genes represented in 75 probe-sets that showed significant differential expression between paired hepatocellular carcinoma and adjacent non-tumorous liver tissues. The gene for which the expression intensities are indicated is shown in the top left corner of each graph. Each of FIGS. 6-10 contain 15 graphs showing the expression intensities of individual genes represented in the 75 probe-sets. Expression intensities are shown for non-cancerous liver tissue (PN) and HCC (PHCC) tissue samples from 18 paired adjacent tissue samples, as well as 82 additional HCC samples (HCC), which were not paired with a corresponding adjacent non-cancerous liver tissue sample.

FIG. 11 is a chart showing t-statistics of gene expression for each of 75 probe sets showing significant differential expression between paired hepatocellular carcinoma and adjacent non-tumorous liver tissues. For each gene, the affymetrix ID number of the corresponding probe-set on the Affymetrix chip (Affymetrix Probe Set ID), the number and percentage of 18 paired HCC and adjacent non-cancerous liver tissue samples showing differential expression of the gene at a stringency level of 12 (Involved sample pairs (%)), the gene symbol, the mean signal intensity of the gene's expression in non-cancerous liver tissue (PN) and HCC (PHCC) tissue samples from 18 paired adjacent tissue samples, as well as in 82 additional HCC samples (HCC), as determined using MAS 5.0 software (MAS 5.0 Signal Intensity), and p-values based on paired t-tests for PN vs. PHCC ((A) vs (B)) and PHCC vs. HCC ((B)vs (C)) are shown.

FIGS. 12-14 are a series of graphs depicting the expression intensities of 39 genes represented in 75 probe-sets that showed significant differential expression between paired hepatocellular carcinoma and adjacent non-tumorous liver tissues, as determined by real time quantitative RT-PCR. The gene for which the expression intensities are indicated is shown in the top left corner of each graph. Expression intensities are shown for normal (PN) and HCC (PHCC) tissue samples from 18 paired adjacent tissue samples.

FIG. 15 lists the results of Ingenuity Pathway analysis of 55 HCC-specific genes represented in 75 probe-sets that showed significant differential expression between paired HCC and non-tumorous liver tissue. “Focus Genes” represents the number of the submitted genes that are included in the identified networks of indicated top functions. “Score” was generated by the Ingenuity Pathway software without important significance.

FIG. 16 is a graph depicting the biological functions (x-axis) assigned by Ingenuity pathway analysis to genes represented by 59 tumor-specific probe-sets. Significance levels are expressed as the −log(p-value) along the y-axis. The threshold line is set at 1.301=−log(0.05).

FIG. 17 depicts hierarchical cluster analysis of microarray datasets for HCC (n=100) and non-tumorous liver tissues (n=18). The samples highlighted in gray at the top of the figure are non-tumorous liver tissues. The probe sets highlighted in gray on the left are probe sets that are specific for adjacent non-tumorous liver tissues in 12 out of 18 pairs of HCC and non-tumorous liver tissues (see FIG. 5).

FIG. 18 depicts hierarchical cluster analysis of microarray datasets for nasopharyngeal carcinoma (n=168) and normal nasopharyngeal tissues (n=15). The samples highlighted in gray at the top of the figure are non-tumorous liver tissues. The probe sets highlighted in gray on the left are probe sets that are specific for adjacent non-tumorous liver tissues in 12 out of 18 pairs of HCC and non-tumorous liver tissues (see FIG. 5).

FIG. 19 depicts hierarchical cluster analysis of microarray datasets for breast cancer (n=232) and normal breast tissues (n=25). The datasets used include 207 breast cancer samples from International Genomics Consortium (see Table 3). The samples highlighted in gray at the top of the figure are normal breast tissues. The probe sets highlighted in gray on the left are probe sets that are specific for adjacent non-tumorous liver tissues in 12 out of 18 pairs of HCC and non-tumorous liver tissues (see FIG. 5).

FIG. 20 depicts hierarchical cluster analysis of microarray datasets for lung cancer (n=200) and normal lung tissues (n=15). The datasets used represent 74 lung cancer samples from International Genomic Consortium (see Table 3), 1 1 1 lung cancer samples from Duke University (see Table 3), 15 lung cancer samples and 15 normal lung tissue samples from the Koo Foundation Sun-Yat-Sen Cancer Center (Taipei, Taiwan). The samples highlighted in gray on the top are normal lung tissues. The probe sets highlighted in gray on the left are probe sets that are specific for adjacent non-tumorous liver tissues in 12 out of 18 pairs of HCC and non-tumorous liver tissues (see FIG. 5).

FIG. 21 depicts hierarchical cluster analysis of microarray datasets for colon cancer (n=161) and normal colon tissues (n=15). The datasets represent 146 colon cancer samples from International Genomics Consortium (Table 3), and 15 colon cancer and 15 normal colon tissue samples from the Koo Foundation Sun-Yat-Sen Cancer Center. The samples highlighted in gray on the top are normal colon tissue samples. The probe sets highlighted in gray on the left are probe sets that are specific for adjacent non-tumorous liver tissues in 12 out of 18 pairs of HCC and non-tumorous liver tissues (see FIG. 5).

FIG. 22 depicts hierarchical cluster analysis of microarray datasets for renal cell carcinoma (n=9) and normal kidney tissues (n=8). The dataset was obtained from Boston University (Table 3). The samples highlighted in gray on the top are normal kidney tissue samples. The probe sets highlighted in gray on the left are probe sets that are specific for adjacent non-tumorous liver tissues in 12 out of 18 pairs of HCC and non-tumorous liver tissues (see FIG. 5).

FIG. 23A depicts hierarchical cluster analysis of t-statistics results, comparing gene expression intensities of the 75 selected probe-sets (see FIGS. 4 and 5) between 20 different types of cancer and their corresponding normal tissues from the SCIANTIS™ ProSystem database. The 20 different types of cancers are listed at the top of the figure. The results revealed a cluster of 59 tumor-specific probe-sets with high positive t-values and a cluster of 16 normal tissue-specific probe-sets with negative t-values for all types of cancer tested except for gastrointestinal stromal tumor (GIST) at the right end of the figure. Gray represents t-values of +9, white represents t-values of 0 and black represents t-values of −9. Intermediate values are colored accordingly.

FIG. 23B depicts hierarchical cluster analyses of t-statistics results for 75 randomly selected probe-sets using the gene expression data for the same 20 different types of cancer and their corresponding normal tissues from the SCIANTIS™ Pro System as described in FIG. 23A. A disorderly cluster pattern is observed for these randomly selected probes.

FIG. 24 is a graph depicting sorted p-values oft-tests performed using gene expression data obtained from the SCIANTIS™ Pro System database for 20 different types of cancer samples and their corresponding normal tissues using the 75 probe sets listed in FIGS. 4 and 5. Sorted p-values for all seventy-five (75) probe-sets and 20 types of cancer are depicted by the line from the lowest at the left to the highest at the far right end of the graph. For a control, 75 probe-sets were randomly selected 10,000 times and the results of 10,000 random selections were analyzed statistically and plotted as 10,000 lines (shown to the left of the far right line).

FIG. 25 depicts hierarchical cluster analysis of gene expression data from the Gene Expression Omnibus (GEO) dataset for different normal organs and tissues using the 75 probe-sets that showed significant differential expression between paired hepatocellular carcinoma and adjacent non-tumorous liver tissues listed in FIGS. 4 and 5. Twelve lymphoma/leukemia cell lines and two adenocarcinomas of the colon were also included in this dataset. The data set was listed under GEO accession number: GSE1133. The normal tissues/cells on top are bone marrow cells, testicular cells, tonsil and fetal liver. The remaining normal tissues/cells include various parts of brain, spinal cord, adrenal gland, appendix, heart, islet cells, kidney, liver, lung, lymph node, ovary, pancreas, pituitary, prostate, salivary gland, skeletal muscle, skin, thymus, thyroid, tongue, trachea, uterus, whole blood and different subsets of white blood cells (not highlighted).

FIG. 26 depicts a heat map of hierarchical cluster analysis for gene expression data of 100 HCC samples using 75 probe-sets that showed significant differential expression between paired hepatocellular carcinoma and adjacent non-tumorous liver tissues. The gene expression profiling data of 100 HCC samples were generated at the Koo Foundation Sun-Yat-Sen Cancer Center. Group 1 denotes the cluster of HCC samples that showed reduced expression for the 59 tumor-specific probe-sets (see FIG. 4) and Group 2 showed increased expression. The 16 probe-sets that are specific to normal tissues are indicated using light shading.

FIG. 27 depicts a heat map of hierarchical cluster analysis for gene expression data of 168 NPC samples using 75 probe-sets that showed significant differential expression between paired hepatocellular carcinoma and adjacent non-tumorous liver tissues. The gene expression profiling data of 168 NPC samples were generated at the Koo Foundation Sun-Yat-Sen Cancer Center. Group 1 denotes the cluster of NPC samples that showed reduced expression for the 59 tumor-specific probe-sets (see FIG. 4) and Group 2 showed increased expression. The 16 probe-sets that are specific to normal tissues are indicated using light shading.

FIG. 28 depicts a heat map of hierarchical cluster analysis for gene expression data of 295 breast cancer samples from the Netherlands Cancer Institute (NKI) using genes from the 75 probe-sets that could be matched to the NKI breast cancer dataset. The probe-sets that are specific to normal tissues are indicated using light shading. Some genes of the 75 probe-sets are not present in the gene expression profiling dataset of NKI and, therefore, were not included in the hierarchical cluster analysis. Group 1 denotes breast cancer samples that showed reduced expression of tumor-specific probe-sets and Group 2 denotes breast cancer samples that showed increased expression of the same probe-sets. Sample numbers are shown at the top of the figure. The genes matched to the 75 probe-sets are shown on the left. Genes that are specific to normal tissues are indicated using light shading.

FIG. 29A is a graph depicting metastasis-free survival curves for two groups of HCC patients as determined by hierarchical cluster analysis (see FIG. 26). The numbers in parentheses represent events of metastases.

FIG. 29B is a graph depicting overall survival curves for two groups of HCC patients as determined by hierarchical cluster analysis (see FIG. 26). The numbers in parentheses represent events of deaths.

FIG. 30A is a graph depicting metastasis-free survival curves for two groups of breast cancer patients as determined by hierarchical cluster analysis (see FIG. 28). The numbers in parentheses represent events of metastases.

FIG. 30B is a graph depicting overall survival curves for two groups of breast cancer patients as determined by hierarchical cluster analysis (see FIG. 28). The numbers in parentheses represent events of death.

FIG. 31A is a graph depicting metastasis-free survival curves for two groups of nasopharyngeal carcinoma (NPC) patients as determined by hierarchical cluster analysis (see FIG. 27). The numbers in parentheses represent events of metastases.

FIG. 31B is a graph depicting overall survival curves for two groups of nasopharyngeal carcinoma (NPC) patients as determined by hierarchical cluster analysis (see FIG. 27). The numbers in parentheses represent events of death.

FIG. 32 depicts hierarchical clustering analysis of normal testis and adult germ cell tumors with different degrees of differentiation (see key) using the 75 probe-sets that showed significant differential expression between paired hepatocellular carcinoma and adjacent non-tumorous liver tissues. The light background shading on the right indicates a cluster of 16 normal tissue-specific probe-sets. The less differentiated tumors (embryonal carcinomas, yolk sac tumors and seminomas) showed higher expression of tumor-specific probe-sets and less expression of the 16 probe-sets specific to normal tissues than well differentiated tumors (e.g., teratomas).

FIG. 33 is a comparison of three different previously-reported common signatures for cancer (first column: Whitfield M L, et al. Nature Review Cancer 6:99-106 (2006); second and third columns: Rhodes D R, et al. Proc. Nat. Acad. Sci. USA 101:9309-9314 (2004)) with the Common Neoplastic Signature (fourth column) described herein (see Example 1 and FIGS. 4 and 5).

### DETAILED DESCRIPTION OF THE INVENTION

### Definitions

As used herein, “gene expression” refers to the translation of information encoded in a gene into a gene product (e.g., RNA, protein). Expressed genes include genes that are transcribed into RNA (e.g., mRNA) that is subsequently translated into protein, as well as genes that are transcribed into non-coding functional RNA molecules that are not translated into protein (e.g., transfer RNA (tRNA), ribosomal RNA (rRNA), microRNA, ribozymes).

“Level of expression,” “expression level” or “expression intensity” refers to the level (e.g., amount) of one or more products (e.g., mRNA, protein) encoded by a given gene in a sample or reference standard.

As used herein, “differentially expressed” or “differential expression” refers to any statistically significant difference (p<0.05) in the level of expression of a gene between two samples (e.g., two biological samples), or between a sample and a reference standard. Whether a difference in expression between two samples is statistically significant can be determined using an appropriate t-test (e.g., one-sample t-test, two-sample t-test, Welch's t-test) or other statistical test known to those of skill in the art.

As used herein, the phrase “subset of genes overexpressed in cancer” refers to a combination of two or more genes, each of which display an elevated or increased level of expression in a cancer sample relative to a suitable control (e.g., a non-cancerous tissue or cell sample, a reference standard), wherein the elevation or increase in the level of gene expression is statistically-significant (p<0.05). Whether an increase in the expression of a gene in a cancer sample relative to a control is statistically significant can be determined using an appropriate t-test (e.g., one-sample t-test, two-sample t-test, Welch's t-test) or other statistical test known to those of skill in the art. Genes that are overexpressed in a cancer can be, for example, genes that are known, or have been previously determined, to be overexpressed in a cancer.

As used herein, the phrase “subset of genes underexpressed in cancer” refers to a combination of two or more genes, each of which display a reduced or decreased level of expression in a cancer sample relative to a suitable control (e.g., a non-cancerous tissue or cell sample, a reference standard), wherein the reduction or decrease in the level of gene expression is statistically-significant (p<0.05). In some embodiments, the reduced or decreased level of gene expression can be a complete absence of gene expression, or an expression level of zero. Whether a decrease in the expression of a gene in a cancer sample relative to a control is statistically significant can be determined using an appropriate t-test (e.g., one-sample t-test, two-sample t-test, Welch's t-test) or other statistical test known to those of skill in the art. Genes that are underexpressed in a cancer can be, for example, genes that are known, or have been previously determined, to be underexpressed in a cancer.

A “gene expression profile” or “expression profile” refers to a set of genes which have expression levels that are associated with a particular biological activity (e.g., cell proliferation, cell cycle regulation, metastasis), cell type, disease state (e.g., cancer), state of cell differentiation or condition.

A “common neoplastic signature” or “CNS” refers to a gene expression profile that is associated with (e.g., is diagnostic of) many different common cancers.

“Tumor-specific genes” as used herein are genes which have expression levels that are characterized as “present” in a cancer (e.g., a hepatocellular carcinoma) tissue sample, and “absent” or “marginal” in an adjacent non-tumor tissue (e.g., normal liver tissue) sample, by both Affymetrix Microarray Analysis Suite (MAS) 5.0 and DNA Chip Analyzer (dChip) software applications.

“Non-tumor tissue-specific genes” as used herein are genes which have expression levels that are characterized as “absent” or “marginal” in a cancer (e.g., a hepatocellular carcinoma) tissue sample, and “present” in an adjacent non-tumor tissue (e.g., normal liver tissue) sample, by both MAS 5.0 and dChip software applications.

The term “stringency,” “stringency filter,” or “stringency level” as used herein refers to a number that directly corresponds to the number, out of a total of 18, of paired HCC and adjacent non-tumorous liver tissue samples that display significant differential expression of a particular gene or group of genes by microarray expression profiling analysis, as determined by both Affymetrix Microarray Analysis Suite (MAS) 5.0 and DNA Chip Analyzer (dChip) software applications using “present” vs “absent” or “marginal” status. Thus, the values for a “stringency,” “stringency filter,” or “stringency level” used herein range from a high stringency of eighteen to a low stringency of one.

The term “probe set” refers to probes on an array (e.g., a microarray) that are complementary to the same target gene or gene product. A probe set may consist of one or more probes.

As used herein, the term “sample” refers to a biological sample (e.g., a tissue sample, a cell sample, a fluid sample) that expresses genes that display differential levels of expression when cancer cells are present in the sample versus when cancer cells are absent from the sample, for a given type of cancer.

As used herein, “adjacent samples,” “adjacent tissue samples,” “paired samples” or “paired tissue samples” refer to two or more biological samples that are present in, or isolated from, the same tissue or organ of a subject.

The term “oligonucleotide” as used herein refers to a nucleic acid molecule (e.g., RNA, DNA) that is about 5 to about 150 nucleotides in length. The oligonucleotide may be a naturally occurring oligonucleotide or a synthetic oligonucleotide. Oligonucleotides may be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

As used herein, “probe oligonucleotide” or “probe oligodeoxynucleotide” refers to an oligonucleotide that is capable of hybridizing to a target oligonucleotide.

“Target oligonucleotide” or “target oligodeoxynucleotide” refers to a molecule to be detected (e.g., via hybridization).

“Distant metastasis” refers to cancer cells that have spread from the original (i.e., primary) tumor to distant organs or distant lymph nodes.

“Detectable label” as used herein refers to any moiety that is capable of being specifically detected, either directly or indirectly, and therefore, can be used to distinguish a molecule that comprises the detectable label from a molecule that does not comprise the detectable label.

The phrase “specifically hybridizes” refers to the specific association of two complementary nucleotide sequences (e.g., DNA, RNA or a combination thereof) in a duplex under stringent conditions. The association of two nucleic acid molecules in a duplex occurs as a result of hydrogen bonding between complementary base pairs.

“Stringent conditions” or “stringency conditions” refer to a set of conditions under which two complementary nucleic acid molecules can hybridize. However, stringent conditions do not permit hybridization of two nucleic acid molecules that are not complementary (two nucleic acid molecules that have less than 70% sequence complementarity).

As used herein, “low stringency conditions” include, for example, hybridization in 6× sodium chloride/sodium citrate (SSC) at about 45° C., followed by two washes in 0.2×SSC, 0.1% SDS at least at 50° C. (the temperature of the washes can be increased to 55° C. for low stringency conditions).

“Medium stringency conditions” include, for example, hybridization in 6×SSC at about 45° C., followed by one or more washes in 0.2×SSC, 0.1% SDS at 60° C.

As used herein, “high stringency conditions” include, for example, hybridization in 6×SSC at about 45° C., followed by one or more washes in 0.2×SSC, 0.1% SDS at 65° C.;

“Very high stringency conditions” include, but are not limited to, hybridization in 0.5M sodium phosphate, 7% SDS at 65° C., followed by one or more washes at 0.2×SSC, 1% SDS at 65° C.

As used herein, the term “polypeptide” refers to a polymer of amino acids of any length and encompasses proteins, peptides, and oligopeptides.

As used herein, the term “antibody” refers to a polypeptide having affinity for a target, antigen, or epitope, and includes both naturally-occurring and engineered antibodies. The term “antibody” encompasses polyclonal, monoclonal, human, chimeric, humanized, primatized, veneered, and single chain antibodies, as well as fragments of antibodies (e.g., Fv, Fc, Fd, Fab, Fab′, F(ab′), scFv, scFab, dAb). (See e.g., Harlow et al., Antibodies A Laboratory Manual, Cold Spring Harbor Laboratory, 1988).

As defined herein, the term “antigen binding fragment” refers to a portion of an antibody that contains one or more CDRs and has affinity for an antigenic determinant by itself. Non-limiting examples include Fab fragments, F(ab)′₂ fragments, heavy-light chain dimers, and single chain structures, such as a complete light chain or a complete heavy chain.

As used herein, “specifically binds” refers to a probe (e.g., an antibody, an aptamer) that binds to a target protein (e.g., the protein product of a CNS gene) with an affinity (e.g., a binding affinity) that is at least about 5 fold, preferably at least about 10 fold, greater than the affinity with which the probe binds a non-target protein.

“Target protein” refers to a protein to be detected (e.g., using a probe comprising a detectable label).

As used herein, a “subject” refers to a mammal. The term “subject” therefore, includes, for example, primates (e.g., humans), cows, sheep, goats, horses, dogs, cats, rabbits, guinea pigs, rats, mice or other bovine, ovine, equine, canine, feline, rodent or murine species. In a preferred embodiment, the subject is a human. Examples of suitable subjects include, but are not limited to, human patients that have, or are at risk for developing, a cancer (e.g., HCC).

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 2d ed. (1989) Cold Spring Harbor Laboratory Press, Cold Spring Harbor, N.Y. and Ausubel et al., Short Protocols in Molecular Biology (1999) 4th Ed, John Wiley & Sons, Inc. which are incorporated herein by reference) and chemical methods.

As described herein, a gene expression profile that includes genes that are differentially expressed between paired hepatocellular carcinoma (HCC) and normal liver tissues can serve as a common neoplastic signature (“CNS”) that is capable of differentiating several different types of cancers from corresponding normal tissues. As described herein, a common neoplastic signature of 55 genes was able to distinguish tissue samples representing six major types of cancers, and 19 out of 20 subtypes of cancers, from corresponding normal tissue samples. In addition, a subset of the genes in the CNS were associated with poor prognoses, including shorter survival or increased risk of distant metastasis, for three different types of cancer (HCC, nasopharyngeal cancer and breast cancer).

### Diagnostic and Prognostic Methods

The present invention encompasses, in one embodiment, a method of diagnosing whether a subject has a cancer. The method comprises detecting in a sample from the subject the level of expression of a subset of genes that are overexpressed in the cancer (e.g., tumor). Increased levels of expression of the genes of the subset in the sample from the subject, relative to a control, indicate that the subject has cancer.

The subset of genes that are overexpressed in the cancer can include any combination of two or more genes from a common neoplastic signature that includes the following 55 genes: MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARP1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1 and NUSAP1. The gene known in the art as HCAP-G is also known in the art as NCAPG, and these two gene designations are used interchangeably herein.

Different subsets of genes from the CNS are likely to be overexpressed in different cancers (e.g., hepatocellular carcinoma, nasopharyngeal cancer, breast cancer, lung cancer, renal cell carcinoma, colon cancer). Therefore, the particular genes and/or number of genes in the CNS that are overexpressed in a given type or subtype of cancer may differ from the genes and/or number of genes from the CNS that are overexpressed in another type or subtype of cancer. The subset of genes that are overexpressed in a cancer can include 2 or more genes of the CNS, up to, and including all 55 genes of the CNS described herein. In one embodiment, the subset of genes that are overexpressed in a cancer includes all 55 genes of the common neoplastic signature. In another embodiment, the subset of genes that are overexpressed in a cancer includes about 20 genes of the CNS. The nucleotide sequences of the genes of the common neoplastic signature and the nucleotide and amino acid sequences of their RNA and protein products, respectively, have been reported (see Table 1) and can be readily ascertained by those of skill in the art.

TABLE 1
Gene Symbols and GenBank ® Accession Numbers
for Genes in the Common Neoplastic Signature

| | GenBank ® Accession | | GenBank ® Accession |
|---|---|---|---|
| Gene Symbol | Number | Gene Symbol | Number |
| MELK | NM_014791 | CHEK1 | NM_001274 |
| PLVAP | NM_031310 | KIF2C | NM_006845 |
| TOP2A | NM_001067 | AURKB | NM_004217 |
| NEK2 | NM_002497 | NPEPPS | NM_006310 |
| CDKN3 | NM_005192 | KIF4A | NM_012310 |
| PRC1 | NM_199413, NM_003981, | E2F8 | NM_024680 |
| | NM_199414 | | |
| ESM1 | NM_007036 | EZH2 | NM_004456, NM_152998 |
| PTTG1 | NM_004219 | ZNF193 | NM_006299 |
| TTK | NM_003318 | ILF3 | NM_004516, NM_153464, |
| | | | NM_012218 |
| CENPF | NM_016343 | EHMT2 | NM_025256, NM_006709 |
| RDBP | NM_002904 | SF3A2 | NM_007165 |
| CCHCR1 | NM_019052 | NPAS2 | NM_002518 |
| DEPDC1 | NM_017779 | PSME3 | NM_005789, NM_176863 |
| TP53i3 | NM_004881, NM_147184 | INPPL1 | NM_001567 |
| CCNB2 | NM_004701 | BIRC5 | NM_001012271, |
| | | | NM_001168 |
| CAD | NM_004341 | SULT1C2 | NM_001056, NM_176825 |
| CDC2 | NM_001786, NM_033379 | NSUN5B | NM_145645, |
| | | | NM_001039575 |
| HMMR | NM_012484, NM_012485 | HN1 | NM_017617, |
| | | | NM_001002033, |
| | | | NM_001002032 |
| STMN1 | NM_005563, NM_203401, | NUSAP1 | NM_018454, NM_016359 |
| | NM_203399 | | |
| NCAPG | NM_022346 | NAT2 | NM_000015 |
| MDK | NM_002391, | CD5L | NM_005894 |
| | NM_001012333, | | |
| | NM_001012334 | | |
| RAD54B | NM_012415 | CXCL14 | NM_004887 |
| ASPM | NM_018136 | VIPR1 | NM_004624 |
| HMGA1 | NM_145902, NM_145903 | CCL14, CCI15 | NM_032963, NM_004166, |
| | | | NM_032964, NM_032965 |
| SNRPC | NM_003093 | FCN3 | NM_003665, NM_173452 |
| IGF2BP3 | NM_006547 | CRHBP | NM_001882 |
| SERPINH1 | NM_001235 | GPD1 | NM_005276 |
| COL4A1 | NM_001845 | KCNN2 | NM_021614, NM_170775 |
| LARP1 | NM_015315, NM_033551 | HGFAC | NM_001528. |
| LRRC1 | NM_018214 | FOSB | NM_006732 |
| FOXM1 | NM_021953, NM_202003, | LCAT | NM_000229 |
| | NM_202002 | | |
| CDC20 | NM_001255 | MARCO | NM_006770 |
| UBE2M | NM_003969 | CYP1A2 | NM_000761 |
| DNAJC6 | NM_014787 | FCN2 | NM_004108, NM_015837 |
| FEN1 | NM_004111 | DPT | NM_001937 |
| ASNS | NM_183356, NM_133436, | | |
| | NM_001673 | | |

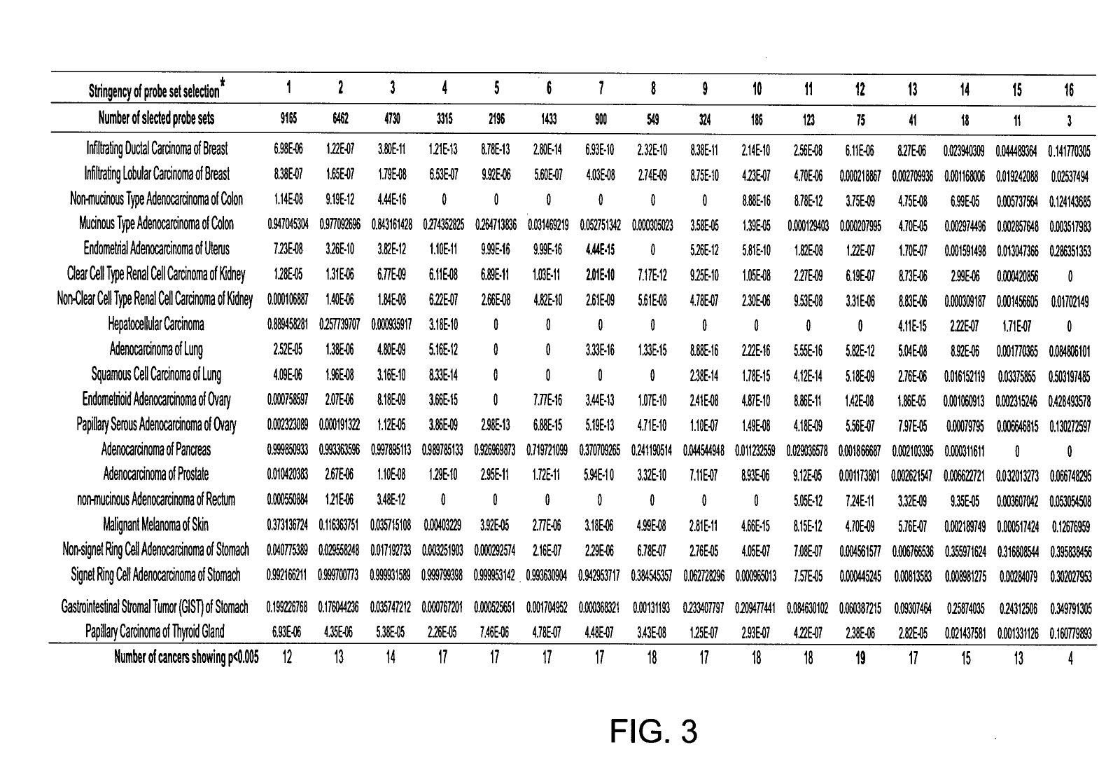

The methods described herein can be used to diagnose many different types of cancers. In a particular embodiment, the methods of the invention can be used to diagnose a cancer selected from the group consisting of breast cancer, colon cancer, endometrial cancer, renal cell carcinoma, liver cancer, lung cancer, ovarian cancer, pancreatic cancer, prostate cancer, rectal cancer, skin cancer, stomach cancer, and thyroid cancer. Various cancer subtypes can also be diagnosed using the methods of the inventions. Such cancer subtypes include, but are not limited to the cancer subtypes listed in FIG. 3. In a preferred embodiment, the cancer is hepatocellular carcinoma. Not all of the genes in the common neoplastic signature identified herein will have expression levels that are associated with (e.g., are diagnostic of) every type or subtype of cancer described herein. Thus, different types or subtypes of cancer may be diagnosed using various subsets of the CNS genes identified herein.

In another embodiment, the invention relates to a method of providing a prognosis for a subject that has a cancer, comprising detecting the level of expression of one or more genes of the CNS. According to the invention, expression (e.g., overexpression) of certain genes in the CNS is indicative of a poor prognosis. The prognosis can be, but is not limited to, a prognosis for patient survival, risk of metastases, or risk of relapse after treatment. In a particular embodiment, the prognosis is for a patient that has hepatocellular carcinoma, nasopharyngeal cancer or breast cancer.

As described herein, a strong association exists between expression (e.g., overexpression) of certain genes in the CNS in cancer samples and a poor patient prognosis (e.g., shorter survival, increased risk of metastases (see, e.g., Examples 4-7)). Specifcally, expression (e.g., elevated expression) of PRC1, CENPF, RDBP, CCNB2 and/or RAD54B in samples from subjects that have hepatocellular carcinoma, nasopharyngeal cancer or breast cancer, is associated with an increased risk of distant metastasis. In addition, expression (e.g., elevated expression) of CDC2, CCHCR1, and/or HMGA1 in samples from subjects that have hepatocellular carcinoma, nasopharyngeal cancer or breast cancer, is associated with a shorter survival.

For the diagnostic and prognostic methods of the invention, gene expression can be assessed in a suitable sample from a subject. A suitable sample can be a tissue sample, a biological fluid sample, a cell (e.g., a tumor cell) sample, and the like. Any means of sampling from a subject, for example, by blood draw, spinal tap, tissue smear or scrape, or tissue biopsy can be used to obtain a sample. Thus, the sample can be a biopsy specimen (e.g., tumor, polyp, mass (solid, cell)), aspirate, smear or blood sample. In a preferred embodiment, the sample is a blood sample (e.g., a blood serum sample). The sample can be a tissue from an organ that has a tumor (e.g., cancerous growth) and/or tumor cells, or is suspected of having a tumor and/or tumor cells. For example, a tumor biopsy can be obtained in an open biopsy, a procedure in which an entire (excisional biopsy) or partial (incisional biopsy) mass is removed from a target area. Alternatively, a tumor sample can be obtained through a percutaneous biopsy, a procedure performed with a needle-like instrument through a small incision or puncture (with or without the aid of an imaging device) to obtain individual cells or clusters of cells (e.g., a fine needle aspiration (FNA)) or a core or fragment of tissues (core biopsy). The biopsy samples can be examined cytologically (e.g., smear), histologically (e.g., frozen or paraffin section) or using any other suitable method (e.g., molecular diagnostic methods). A tumor sample can also be obtained by in vitro harvest of cultured human cells derived from an individual's tissue. Tumor samples can, if desired, be stored before analysis by suitable storage means that preserve a sample's protein and/or nucleic acid in an analyzable condition, such as quick freezing, or a controlled freezing regime. If desired, freezing can be performed in the presence of a cryoprotectant, for example, dimethyl sulfoxide (DMSO), glycerol, or propanediol-sucrose. Tumor samples can be pooled, as appropriate, before or after storage for purposes of analysis.

In one embodiment, a cancer can be diagnosed, or a prognosis for a subject can be provided, by detecting expression of a subset of genes from the CNS, or their gene products (e.g., mRNA, protein), in a sample from a patient. Thus, the method does not require that expression in the sample from the patient be compared to a control. The presence or absence of gene expression can be ascertained by the methods described herein or other suitable assays known to those of skill in the art.

A difference (e.g., an increase, a decrease) in gene expression can be determined by comparison of the level of expression of the gene in a sample from a subject to that of a suitable control. Suitable controls include, for instance, a non-neoplastic tissue sample (e.g., a non-neoplastic tissue sample from the same subject from which the cancer sample has been obtained), a sample of non-cancerous cells, non-metastatic cancer cells, non-malignant (benign) cells or the like, or a suitable known or determined reference standard. The reference standard can be a typical, normal or normalized range of levels, or a particular level, of expression of a protein or RNA (e.g., an expression standard). The standards can comprise, for example, a zero gene expression level, the gene expression level in a standard cell line, or the average level of gene expression previously obtained for a population of normal human controls. Thus, the method does not require that expression of the gene/gene product be assessed in, or compared to, a control sample.

Suitable assays that can be used to assess the level of expression of a gene, or the level (e.g., amount) of a gene product (e.g., mRNA, protein), in a sample (e.g., biological sample) from a subject are known to those of skill in the art. For example, the level of an RNA (e.g., mRNA) gene product in a sample can be measured using any technique that is suitable for detecting RNA expression levels in a biological sample. Several suitable techniques for determining RNA expression levels in cells from a biological sample (e.g., Northern blot analysis, RT-PCR, in situ hybridization) are well known to those of skill in the art. In a particular embodiment, the level of at least one gene product is detected using Northern blot analysis. For example, total cellular RNA can be purified from cells by homogenization in the presence of nucleic acid extraction buffer, followed by centrifugation. Nucleic acids are precipitated, and DNA is removed by treatment with DNase and precipitation. The RNA molecules are then separated by gel electrophoresis on agarose gels according to standard techniques, and transferred to nitrocellulose filters. The RNA is then immobilized on the filters by heating. Detection and quantification of specific RNA is accomplished using appropriately labeled DNA or RNA probes complementary to the RNA in question. See, for example, Molecular Cloning: A Laboratory Manual, J. Sambrook et al., eds., 2nd edition, Cold Spring Harbor Laboratory Press, 1989, Chapter 7, the entire disclosure of which is incorporated by reference.

Suitable probes for Northern blot hybridization include nucleic acid probes that are complementary to the nucleotide sequences of the RNA (e.g., mRNA) and/or cDNA sequences of the genes of the CNS. Methods for preparation of labeled DNA and RNA probes, and the conditions for hybridization thereof to target nucleotide sequences, are described in Molecular Cloning: A Laboratory Manual, J. Sambrook et al., eds., 2nd edition, Cold Spring Harbor Laboratory Press, 1989, Chapters 10 and 11, the disclosures of which are herein incorporated by reference.

For example, the nucleic acid probe can be labeled with, e.g., a radionuclide such as ³H, ³²P, ³³P, ¹⁴C, or ³⁵S; a heavy metal; or a ligand capable of functioning as a specific binding pair member for a labeled ligand (e.g., biotin, avidin or an antibody), a fluorescent molecule, a chemiluminescent molecule, an enzyme or the like.

Probes can be labeled to high specific activity by either the nick translation method of Rigby et al. (1977), J. Mol. Biol. 113:237-251 or by the random priming method of Fienberg et al. (1983), Anal. Biochem. 132:6-13, the entire disclosures of which are herein incorporated by reference. The latter is the method of choice for synthesizing ³²P-labeled probes of high specific activity from single-stranded DNA or from RNA templates. For example, by replacing preexisting nucleotides with highly radioactive nucleotides according to the nick translation method, it is possible to prepare ³²P-labeled nucleic acid probes with a specific activity well in excess of 10⁸ cpm/microgram. Autoradiographic detection of hybridization can then be performed by exposing hybridized filters to photographic film. Densitometric scanning of the photographic films exposed by the hybridized filters provides an accurate measurement of gene transcript levels. Using another approach, gene transcript levels can be quantified by computerized imaging systems, such the Molecular Dynamics 400-B 2D Phosphorimager available from Amersham Biosciences, Piscataway, N.J.

Where radionuclide labeling of DNA or RNA probes is not practical, the random-primer method can be used to incorporate an analogue, for example, the dTTP analogue 5-(N—(N-biotinyl-epsilon-aminocaproyl)-3-aminoallyl)deoxyuridine triphosphate, into the probe molecule. The biotinylated probe oligonucleotide can be detected by reaction with biotin-binding proteins, such as avidin, streptavidin, and antibodies (e.g., anti-biotin antibodies) coupled to fluorescent dyes or enzymes that produce color reactions.

In addition to Northern and other RNA hybridization techniques, determining the levels of RNA transcripts can be accomplished using the technique of in situ hybridization. This technique requires fewer cells than the Northern blotting technique, and involves depositing whole cells onto a microscope cover slip and probing the nucleic acid content of the cell with a solution containing radioactive or otherwise labeled nucleic acid (e.g., cDNA or RNA) probes. This technique is particularly well-suited for analyzing tissue biopsy samples from subjects. The practice of the in situ hybridization technique is described in more detail in U.S. Pat. No. 5,427,916, the entire disclosure of which is incorporated herein by reference. Suitable probes for in situ hybridization of a given gene product can be produced, for example, from the nucleic acid sequences of the RNA products of the CNS genes described herein.

Levels of a nucleic acid (e.g., mRNA transcript) in a sample from a subject can also be assessed using any standard nucleic acid amplification technique, such as, for example, polymerase chain reaction (PCR) (e.g., direct PCR, quantitative real time PCR (qRT-PCR), reverse transcriptase PCR (RT-PCR)), ligase chain reaction, self sustained sequence replication, transcriptional amplification system, Q-Beta Replicase, or the like, and visualized, for example, by labeling of the nucleic acid during amplification, exposure to intercalating compounds/dyes, probes, etc. In a particular embodiment, the relative number of gene transcripts in a sample is determined by reverse transcription of gene transcripts (e.g., mRNA), followed by amplification of the reverse-transcribed products by polymerase chain reaction (e.g., RT-PCR). The levels of gene transcripts can be quantified in comparison with an internal standard, for example, the level of mRNA from a “housekeeping” gene present in the same sample. A suitable “housekeeping” gene for use as an internal standard includes, e.g., myosin or glyceraldehyde-3-phosphate dehydrogenase (G3PDH). The methods for quantitative RT-PCR and variations thereof are within the skill in the art.

In some instances, it may be desirable to simultaneously determine the expression level of several different gene products in a sample. For example, it may be desirable to determine the expression level of the transcripts of all genes in the

CNS described herein in a sample from a subject. Assessing cancer-specific expression levels for many genes individually is time consuming and requires a large amount of total RNA (at least about 20 μg for each Northern blot) and autoradiographic techniques that require radioactive isotopes. To overcome these limitations, an oligolibrary, in microchip format (e.g., a gene chip, a microarray), may be constructed containing a set of probe oligodeoxynucleotides that are specific for a set of genes. Using such a microarray, the expression level of multiple RNA transcripts in a biological sample can be determined by reverse transcribing the RNAs to generate a set of target oligodeoxynucleotides, and hybridizing them to probe oligodeoxynucleotides on the microarray to generate a hybridization, or expression, profile. The hybridization profile of the test sample can then be compared to that of a control sample to determine which RNAs have an altered expression level in a cancer sample.

The microarray may be fabricated using techniques known in the art. For example, probe oligonucleotides of an appropriate length can be 5′-amine modified at position C6 and printed using commercially available microarray systems, e.g., the GeneMachine OmniGrid™ 100 Microarrayer and Amersham CodeLink™ activated slides. Labeled cDNA oligomers corresponding to the target RNAs are prepared by reverse transcribing the target RNA with labeled primer. Following first strand synthesis, the RNA/DNA hybrids are denatured to degrade the RNA templates. The labeled target cDNAs thus prepared are then hybridized to the microarray chip under hybridizing conditions, e.g. 6×SSPE/30% formamide at 25° C. for 18 hours, followed by washing in 0.75× TNT at 37° C. for 40 minutes. At positions on the array where the immobilized probe DNA recognizes a complementary target cDNA in the sample, hybridization occurs. The labeled target cDNA marks the exact position on the array where binding occurs, allowing automatic detection and quantification. The output consists of a list of hybridization events, indicating the relative abundance of specific cDNA sequences, and therefore the relative abundance of the corresponding gene products, in the patient sample. According to one embodiment, the labeled cDNA oligomer is a biotin-labeled cDNA, prepared from a biotin-labeled primer. The microarray is then processed by direct detection of the biotin-containing transcripts using, e.g., Streptavidin-Alexa647 conjugate, and scanned utilizing conventional scanning methods. Images intensities of each spot on the array are proportional to the abundance of the corresponding gene product in the patient sample.

An “expression profile” or “hybridization profile” of a particular sample is essentially a fingerprint of the state of the sample; while two states may have any particular genes similarly expressed, the evaluation of a number of genes simultaneously allows the generation of a gene expression profile that is unique to the state of the cell. That is, normal tissue may be distinguished from cancer tissue, and within cancer tissue, different prognosis states (good or poor long term survival prospects, for example) may be determined. By comparing expression profiles of cancer tissue in different states, information regarding which genes are important (including both up- and down-regulation of genes) in each of these states is obtained. The identification of sequences that are differentially expressed in cancer tissue versus normal tissue, as well as differential expression resulting in different prognostic outcomes, allows the use of this information in a number of ways. For example, a particular treatment regime may be evaluated (e.g., to determine whether a chemotherapeutic drug act to improve the long-term prognosis in a particular patient). Similarly, diagnosis may be done or confirmed by comparing patient samples with the known expression profiles. Furthermore, these gene expression profiles (or individual genes) allow screening of drug candidates that suppress the breast cancer expression profile or convert a poor prognosis profile to a better prognosis profile.

In a particular embodiment, total RNA from a sample from a subject that has, or is suspected of having or being at risk for developing, a cancer is quantitatively reverse transcribed to provide a set of labeled target oligodeoxynucleotides complementary to the RNA in the sample. The target oligodeoxynucleotides are then hybridized to a microarray comprising gene-specific probe oligonucleotides to provide a hybridization profile for the sample. The result is a hybridization profile for the sample representing the expression pattern of genes in the sample. The hybridization profile comprises the signal from the binding of the target oligodeoxynucleotides from the sample to the gene-specific probe oligonucleotides in the microarray. The profile may be recorded as the presence or absence of binding (signal vs. zero signal). More preferably, the profile recorded includes the intensity of the signal from each hybridization. The profile is compared to the hybridization profile generated from a normal, i.e., noncancerous, control sample. An alteration (e.g., increase) in the signal is indicative of the presence of the cancer in the subject.

Gene expression on an array or gene chip can be assessed using an appropriate algorithm (e.g., statistical algorithm). Suitable software applications for assessing gene expression levels using a microarray or gene chip are known in the art. In a particular embodiment, gene expression on a microarray is assessed using Affymetrix Microarray Analysis Suite (MAS) 5.0 software and/or DNA Chip Analyzer (dChip) software, for example, as described herein in Example 1.

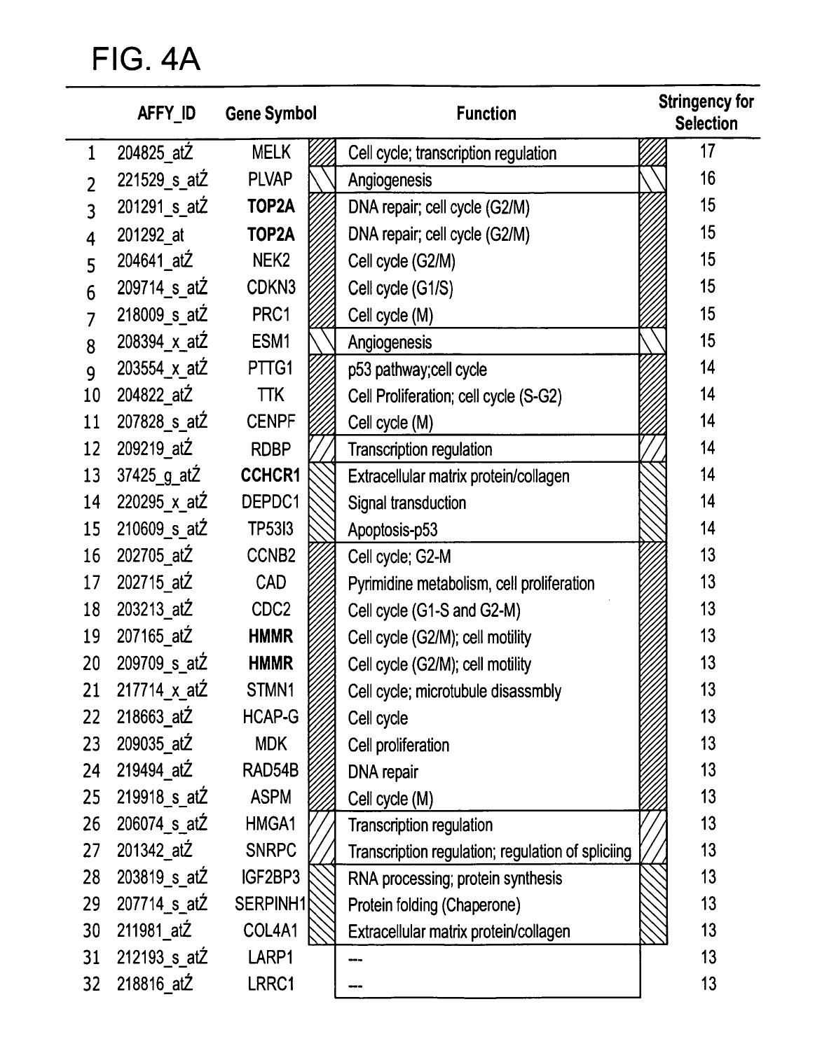

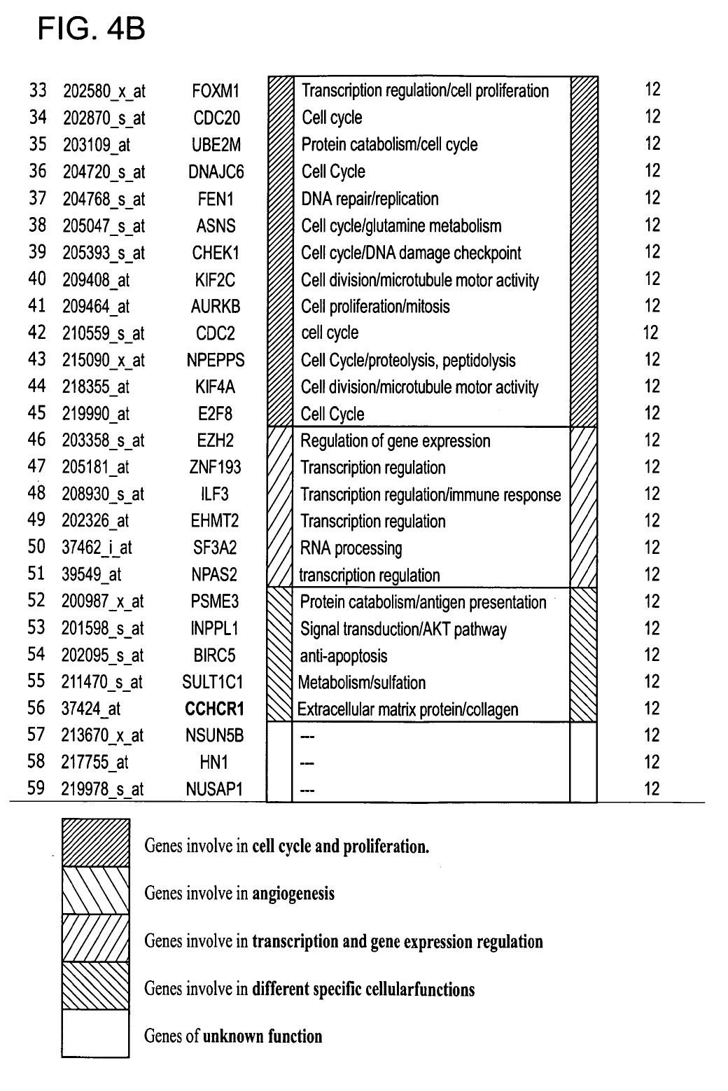

In a particular embodiment, fragments of RNA transcripts for any of the 55 tumor-specific genes described herein (see FIG. 4) can be identified in the blood (e.g., blood plasma) or other bodily fluids (e.g., blood or other body fluids that contain cancer cells) of a subject and quantified, e.g., by performing reverse transcription, PCR and parallel sequencing as described by Palacios G, et al., New Eng. J. Med. 358: 991-998 (2008). The identity of any RNA fragment can be determined by matching its sequence to one of the cDNA sequences of the 55 tumor specific genes. RNA fragments of the 55 tumor-specific genes can also be quantified according to the frequency with which a fragment having a particular DNA sequence from among the 55 tumor-specific genes is detected among all the sequenced PCR fragments from the sample. This approach can be used to screen and identify subjects that are positive for cancer cells. Alternatively, the identities of fragments of RNA transcripts for any of the 55 tumor-specific genes in a blood or biological fluid sample from a subject can be determined and quantified, for example, by performing reverse transcription of the RNA fragment(s), followed by PCR amplification and hybridization of the PCR product(s) to an array (e.g., a microarray, a gene chip).

Other techniques for measuring gene expression in a sample are also within the skill in the art, and include various techniques for measuring rates of RNA transcription and degradation.

The level of expression of a gene of the CNS can also be determined by assessing the level of a protein(s) encoded by the gene in a sample from a subject. Methods for detecting a protein product of a CNS gene include, for example, immunological and immunochemical methods, such as flow cytometry (e.g., FACS analysis), enzyme-linked immunosorbent assays (ELISA), chemiluminescence assays, radioimmunoassay, immunoblot (e.g., Western blot), immunohistochemistry (IHC), and mass spectrometry. For instance, antibodies to a protein product of a CNS gene can be used to determine the presence and/or expression level of the protein in a sample either directly or indirectly e.g., using immunohistochemistry (IHC). For example, paraffin sections can be taken from a biopsy, fixed to a slide and combined with one or more antibodies by suitable methods.

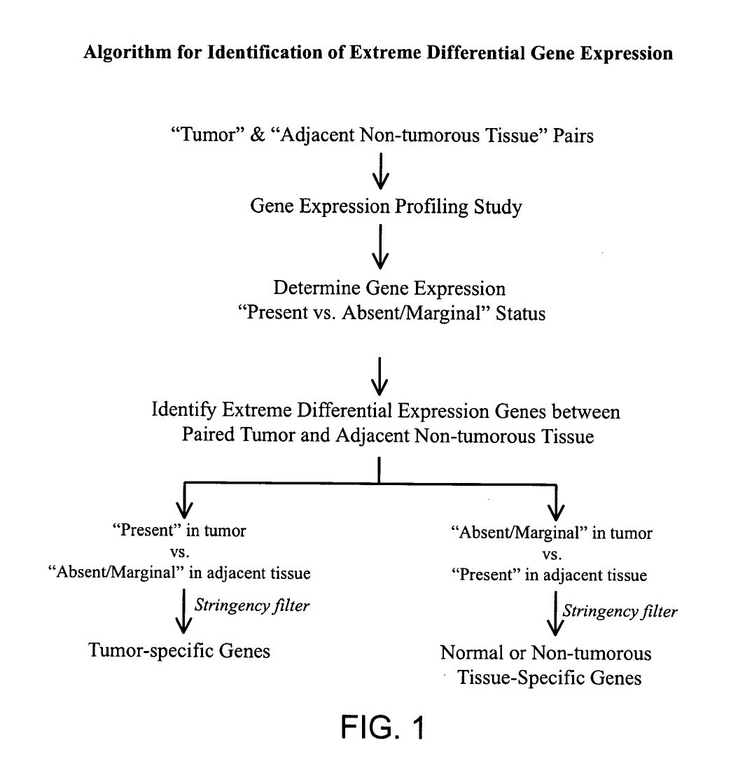

A difference (e.g., an increase, a decrease) in the level of expression of a gene between two samples, or between a sample and a reference standard, can be determined using an appropriate algorithm, several of which are know to those of skill in the art. For example, the identification of genes displaying differential expression (e.g., significant differential expression) between cancer (e.g., HCC) and adjacent non-tumor tissues, can be determined using the algorithm described herein in Example 1 and FIG. 1.

A statistically significant difference (e.g., an increase, a decrease) in the level of expression of a gene between two samples, or between a sample and a reference standard, can be determined using an appropriate statistical test(s), several of which are known to those of skill in the art. In a particular embodiment, a t-test (e.g., a one-sample t-test, a two-sample t-test) is employed to determine whether a difference in gene expression is statistically significant. For example, a statistically significant difference in the level of expression of a gene between two samples can be determined using a two-sample t-test (e.g., a two-sample Welch's t-test). A statistically significant difference in the level of expression of a gene between a sample and a reference standard can be determined using a one-sample t-test. Other useful statistical analyses for assessing differences in gene expression include a Chi-square test, Fisher's exact test, and log-rank and Wilcoxon tests (see Examples 1-7).

### Kits

The present invention also encompasses kits for diagnosing whether a subject has a cancer. Diagnostic kits of the invention include a collection of probes capable of detecting the level of expression of multiple genes of the CNS described herein (i.e., MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARD 1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1, NUSAP1). For example, the kits can include a collection of probes capable of detecting the level of expression of at least about two genes of the CNS, for example about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, or 55 genes of the common neoplastic signature. In one embodiment, the kit encompasses a collection of probes capable of detecting the level of expression of all 55 genes in the common neoplastic signature. In a particular embodiment, the kits encompass a collection of probes capable of detecting the level of expression of at least about ten (10) genes, preferably about fifteen (15) genes, and more preferably, about twenty (20) genes of the CNS described herein.

The invention also provides kits for determining the prognosis (e.g., risk of metastasis, survival) of a subject that has a cancer. In one embodiment, the kits comprise a probe that is capable of detecting the level of expression of at least one gene selected from the group consisting of PRC1, CENPF, RDBP, CCNB2 and RAD54B, or any combination thereof. In another embodiment, the invention relates to kits for determining the prognosis of a subject that has a cancer, comprising a probe that is capable of detecting the level of expression of at least one gene selected from the group consisting of PRC1, CDC2, CCHCR1 and HMGA1, or any combination thereof.

The diagnostic and prognostic kits of the invention include probes (e.g., nucleic acid probes, antibodies) for detecting the expression of CNS genes in a sample (e.g., a biological sample from a mammalian subject).

Accordingly, in one embodiment, the kit comprises nucleic acid probes (e.g., oligonucleotide probes, polynucleotide probes) that specifically hybridize to an RNA transcript (e.g., mRNA, hnRNA) of a CNS gene. Such probes are capable of binding (i.e., hybridizing) to a target nucleic acid of complementary sequence through one or more types of chemical bonds, usually through complementary base pairing via hydrogen bond formation. As used herein, a nucleic acid probe may include natural (i.e., A, G, U, C or T) or modified bases (7-deazaguanosine, inosine, etc.). In addition, the bases in the nucleic acid probes may be joined by a linkage other than a phosphodiester bond, so long as the linkage does not interfere with hybridization. Thus, probes may be peptide nucleic acids in which the constituent bases are joined by peptide bonds rather than phosphodiester linkages.

Guidance for performing hybridization reactions can be found in Current Protocols in Molecular Biology, John Wiley & Sons, N.Y. (1989), 6.3.1-6.3.6, the relevant teachings of which are incorporated herein by reference in their entirety. Suitable hybridization conditions resulting in specific hybridization vary depending on the length of the region of homology, the GC content of the region, and the melting temperature (“Tm”) of the hybrid. Thus, hybridization conditions may vary in salt content, acidity, and temperature of the hybridization solution and the washes. Complementary hybridization between a probe nucleic acid and a target nucleic acid involving minor mismatches can be accommodated by reducing the stringency of the hybridization media to achieve the desired detection of the target nucleic acid. In a particular embodiment, the nucleic acid probes in the kits of the invention are capable of hybridizing to RNA (e.g., mRNA) transcripts of CNS genes under conditions of high stringency.

In another embodiment, the kits include pairs of oligonucleotide primers that are capable of specifically hybridizing to an RNA transcript of a CNS gene, or a corresponding cDNA. Such primers can be used in any standard nucleic acid amplification procedure (e.g., polymerase chain reaction (PCR), for example, RT-PCR, quantitative real time PCR) to determine the level of the RNA transcript in the sample. As used herein, the term “primer” refers to an oligonucleotide, which is complementary to the template polynucleotide sequence and is capable of acting as a point for the initiation of synthesis of a primer extension product. In one embodiment, the primer is complementary to the sense strand of a polynucleotide sequence and acts as a point of initiation for synthesis of a forward extension product. In another embodiment, the primer is complementary to the antisense strand of a polynucleotide sequence and acts as a point of initiation for synthesis of a reverse extension product. The primer may occur naturally, as in a purified restriction digest, or be produced synthetically. The appropriate length of a primer depends on the intended use of the primer, but typically ranges from about 5 to about 200; from about 5 to about 100; from about 5 to about 75; from about 5 to about 50; from about 10 to about 35; from about 18 to about 22 nucleotides. A primer need not reflect the exact sequence of the template but must be sufficiently complementary to hybridize with a template for primer elongation to occur, i.e., the primer is sufficiently complementary to the template polynucleotide sequence such that the primer will anneal to the template under conditions that permit primer extension.

In another embodiment, the kits of the invention include antibodies that specifically bind a protein encoded by a gene of the CNS described herein. Such antibody probes can be polyclonal, monoclonal, human, chimeric, humanized, primatized, veneered, or single chain antibodies, as well as fragments of antibodies (e.g., Fv, Fc, Fd, Fab, Fab′, F(ab′), scFv, scFab, dAb), among others. (See e.g., Harlow et al., Antibodies A Laboratory Manual, Cold Spring Harbor Laboratory, 1988). Antibodies that specifically bind to protein encoded by a gene of the CNS described herein can be produced, constructed, engineered and/or isolated by conventional methods or other suitable techniques (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer '94), Ausubel, F.M. et al., Eds., (John Wiley & Sons: New York, N.Y.), Chapter 11, (1991); Chuntharapai et al., J. Immunol., 152:1783-1789 (1994); Chuntharapai et al. U.S. Pat. No. 5,440,021)). Other suitable methods of producing or isolating antibodies of the requisite specificity can be used, including, for example, methods which select a recombinant antibody or antibody-binding fragment (e.g., dAbs) from a library (e.g., a phage display library), or which rely upon immunization of transgenic animals (e.g., mice). Transgenic animals capable of producing a repertoire of human antibodies are well-known in the art (e.g., Xenomouse® (Abgenix, Fremont, Calif.)) and can be produced using suitable methods (see e.g., Jakobovits et al., Proc. Natl. Acad. Sci. USA, 90: 2551-2555 (1993); Jakobovits et al., Nature, 362: 255-258 (1993); Lonberg et al., U.S. Pat. No. 5,545,806; Surani et al., U.S. Pat. No. 5,545,807; Lonberg et al., WO 97/13852).

Once produced, an antibody specific for a protein encoded by a CNS gene described herein can be readily identified using methods for screening and isolating specific antibodies that are well known in the art. See, for example, Paul (ed.), Fundamental Immunology, Raven Press, 1993; Getzoff et al., Adv. in Immunol. 43:1-98, 1988; Goding (ed.), Monoclonal Antibodies: Principles and Practice, Academic Press Ltd., 1996; Benjamin et al., Ann. Rev. Immunol. 2:67-101, 1984. A variety of assays can be utilized to detect antibodies that specifically bind to proteins encoded by the CNS genes described herein. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radioimmunoassay, radioimmuno-precipitation, enzyme-linked immunosorbent assay (ELISA), dot blot or Western blot assays, inhibition or competition assays, and sandwich assays.

The probes in the diagnostic and prognostic kits of the invention can be conjugated to one or more labels (e.g., detectable labels). Numerous suitable labels for diagnostic probes are known in the art and include any of the labels described herein. Suitable detectable labels for use in the methods of the present invention include, but are not limited to, chromophores, fluorophores, haptens, radionuclides (e.g., ³H, ¹²⁵I, ¹³¹I, ³²P, ³³P, ³⁵S, ¹⁴C, ⁵¹Cr, ³⁶Cl, ⁵⁷Co, ⁵⁸Co, ⁵⁹Fe and ⁷⁵Se), fluorescence quenchers, enzymes, enzyme substrates, affinity tags (e.g., biotin, avidin, streptavidin, etc.), mass tags, electrophoretic tags and epitope tags that are recognized by an antibody (e.g., digoxigenin (DIG), hemagglutinin (HA), myc, FLAG). In certain embodiments, the label is present on the 5 carbon position of a pyrimidine base or on the 3 carbon deaza position of a purine base of a nucleic acid probe.

In a particular embodiment, the label that is conjugated to the probes is a fluorophore. Suitable fluorophores can be provided as fluorescent dyes, including, but not limited to Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), CAL dyes, Carboxyrhodamine 6G, carboxy-X-rhodamine (ROX), Cascade Blue, Cascade Yellow, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, Carboxy-fluorescein (FAM), Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Oyster dyes, Pacific Blue, PyMPO, Pyrene, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, and Texas Red-X.

Probes can also be labeled using fluorescence emitting metals such as ¹⁵²Eu, or others of the lanthanide series. These metals can be attached to the antibody molecule using such metal chelating groups as diethylenetriaminepentaacetic acid (DTPA), tetraaza-cyclododecane-tetraacetic acid (DOTA) or ethylenediaminetetraacetic acid (EDTA).

In addition to the various detectable moieties mentioned above, the probes in the kits of the invention may also be conjugated to other types of labels, such as spectrally resolvable quantum dots, metal nanoparticles or nanoclusters, etc., which may be directly attached to a nucleic acid probe. As mentioned above, detectable moieties need not themselves be directly detectable. For example, they may act on a substrate which is detected, or they may require modification to become detectable.

For in vivo detection, probes may be conjugated to radionuclides either directly or by using an intermediary functional group. An intermediary group which is often used to bind radioisotopes, which exist as metallic cations, to antibodies is diethylenetriaminepentaacetic acid (DTPA) or tetraaza-cyclododecane-tetraacetic acid (DOTA). Typical examples of metallic cations which are bound in this manner are ⁹⁹Tc, ¹²³I, ¹¹¹In, ¹³¹I, ⁹⁷Ru, ⁶⁷Cu, ⁶⁷Ga, and ⁶⁸Ga.

Moreover, probes may be tagged with an NMR imaging agent which include paramagnetic atoms. The use of an NMR imaging agent allows the in vivo diagnosis of the presence of and the extent of the cancer in a patient using NMR techniques. Elements which are particularly useful in this manner are ¹⁵⁷Gd, ⁵⁵Mn, ¹⁶²Dy, ⁵²Cr, and ⁵⁶Fe.

Detection of the labeled probes can be accomplished by a scintillation counter, for example, if the detectable label is a radioactive gamma emitter, or by a fluorometer, for example, if the label is a fluorescent material. In the case of an enzyme label, the detection can be accomplished by colorimetric methods which employ a substrate for the enzyme. Detection may also be accomplished by visual comparison of the extent of the enzymatic reaction of a substrate to similarly prepared standards.

### Methods of Determining Gene Expression Profiles for Cancer

In another embodiment, the invention relates to a method of determining a gene expression profile for a cancer. The method comprises detecting the expression of genes in both cancerous and non-cancerous samples (e.g., tissue samples) from the same individual (see Example 1 below). In a particular embodiment, the cancerous and non-cancerous samples from the same individual are adjacent or paired samples (e.g., adjacent or paired hepatocellular carcinoma and normal liver tissue samples). The expression of genes in a sample can be detected using any suitable gene expression detection method described herein. Moreover, suitable methods for determining differences in gene expression levels between two samples (e.g., adjacent or paired cancer and normal tissue samples) are known to those of skill in the art and include, for example, those described herein. According to the invention, genes that are identified as being differentially expressed between the cancerous and non-cancerous samples are included in the gene expression profile for the cancer.

A description of example embodiments of the invention follows.

### EXEMPLIFICATION

### Example 1

### Identification of Genes Showing Significant Differential Expression Between Paired HCC and Adjacent Non-Tumorous Liver Tissues

### Materials and Methods:

### Tissue Samples

Tissues of HCC and adjacent non-tumorous liver were collected from fresh specimens surgically removed from human patients for therapeutic purpose. These specimens were collected under direct supervision of attending pathologists. The collected tissues were immediately stored in liquid nitrogen at the Tumor Bank of the Koo Foundation Sun Yat-Sen Cancer Center (KF-SYSCC). Paired tissue samples from eighteen HCC patients were available for the study. The study was approved by the Institutional Review Board and written informed consent was obtained from all patients. The clinical characteristics of the eighteen HCC patients from this study are summarized in Table 2.

TABLE 2
Clinical data for eighteen HCC patients from which paired HCC and
adjacent non-tumorous liver tissue samples were obtained

| Case | | | | | | TNM | AFP | |
|---|---|---|---|---|---|---|---|---|
| No. | Sex | Age | HBsAg | HBsAb | HCVIgG | Stage | (ng/ml) | Differentiation |
| 1 | M | 70 | + | | − | 2 | 2 | Moderate |
| 2 | M | 75 | − | + | + | 4A | 5 | Well |
| 3 | M | 59 | + | | − | 4A | 1232 | Moderate |
| 4 | F | 53 | + | | + | 1 | 261 | Moderate |
| 5 | M | 45 | + | | − | 2 | 103 | Moderate |
| 6 | M | 57 | + | + | − | 2 | 5 | Moderate |
| 7 | M | 53 | + | + | − | 3A | 19647 | Moderate |
| 8 | M | 54 | − | − | + | 3A | 7 | Moderate |
| 9 | M | 44 | + | | − | 4A | 306 | Moderate |
| 10 | M | 76 | − | − | + | 3A | 371 | Moderate |
| 11 | F | 62 | + | − | − | 3A | 302 | Moderate |
| 12 | F | 73 | − | − | + | 2 | 42 | Moderate |
| 13 | m | 46 | + | | − | 4A | 563 | Moderate |
| 14 | M | 45 | − | | − | 3A | 64435 | Moderate |
| 15 | M | 41 | + | | − | 2 | 33.9 | Well |
| 16 | M | 44 | + | + | − | 2 | 350 | Moderate |
| 17 | M | 67 | + | | − | 3A | 51073 | Moderate |
| 18 | M | 34 | + | | − | 4A | 2331 | Moderate |

mRNA Transcript Profiling

Total RNA was isolated from tissues frozen in liquid nitrogen using Trizol reagents (Invitrogen, Carlsbad, Calif.). The isolated RNA was further purified using RNAEasy Mini kit (Qiagen, Valencia, Calif.), and its quality assessed using the RNA 6000 Nano assay in an Agilent 2100 Bioanalyzer (Agilent Technologies, Waldbronn, Germany). All RNA samples used for the study had an RNA Integrity Number (RIN) greater than 5.7 (8.2±1.0, mean±SD). Hybridization targets were prepared from 8 μg total RNA according to Affymetrix protocols and hybridized to an Affymetrix U133A GeneChip, which contains 22,238 probe-sets for approximately 13,000 human genes. Immediately following hybridization, the hybridized array underwent automated washing and staining using an Affymetrix GeneChip fluidics station 400 and the EukGE WS2v4 protocol. Thereafter, U133A GeneChips were scanned in an Affymetrix GeneArray scanner 2500.

### Determination of Present and Absent Call of Microarray Data

Affymetrix Microarray Analysis Suite (MAS) 5.0 software was used to generate present calls for the microarray data for all 18 pairs of HCC and adjacent non-tumor liver tissues. All parameters for present call determination were default values. Each probe-set was determined as “present”, “absent” or “marginal” by MAS 5.0. Similarly, the same microarray data were processed using dChip version-2004 software to determine “present”, “absent” or “marginal” status for each probe-set on the microarrays.

Identification of Probe-Sets with Significant Differential Expression

For identification of genes with significant differential expression (i.e., gene expression that is robust in one sample (e.g., an HCC sample), but absent or marginal in an adjacent sample (e.g., a normal liver sample)) between HCC and adjacent non-tumor liver tissues, software written using Practical Extraction and Report Language (PERL) was used according to the following rules: “Tumor-specific genes” were defined as probe-sets that were called “present” in HCC and “absent” or “marginal” in the adjacent non-tumor liver tissue by both MAS 5.0 and dChip. “Non-tumor liver tissue-specific genes” were defined as probe-sets called “absent” or “marginal” in HCC and “present” in the paired adjacent non-tumor liver tissue by both MAS 5.0 and dChip. A flowchart diagram depicting the identification algorithm is shown in FIG. 1.

### Microarray Datasets

In addition to the microarray data collected from the 18 pairs of HCC and adjacent non-tumorous liver tissues, further microarray data were obtained from 82 HCC tissue samples and 168 nasopharyngeal carcinoma (NPC) tissue samples that were collected in a similar manner. The SCIANTIS™ System Pro commercial microarray database (Gene Logic Inc., Gaithersburg, Md.) for various normal and tumor tissues was used for validation purposes. The commercial SCIANTIS™ gene expression datasets are based on Affymetrix HG-U133 A Genechip technology. For a given type of cancer or normal tissue, expression intensity of each probe-set was supplied as mean signal intensity plus standard deviation of a cohort after normalization of gene expression data of each microarray to a global trimmed mean of 100 by MAS 5.0. In addition, microarray datasets from public sources were also used in these studies (Table 3).

TABLE 3
Sources of public-domain microarray datasets.

| | | | GEO |
|---|---|---|---|
| Tissue | Source | Microarray | Accession* |
| Breast cancer | Netherlands Cancer Institute/Stanford | cDNA | — |
| Breast cancer | International Genomics Consortium | U133 plus2 | GSE2109 |
| Lung cancer | International Genomics Consortium | U133 plus2 | GSE2109 |
| Lung cancer | Duke University | U133 plus2 | GSE3141 |
| Renal cell carcinoma | Boston University | U133 A & B | GSE781 |
| Colon cancer | International Genomics Consortium | U133 plus2 | GSE2109 |
| Adult germ cell tumors | Memorial Sloan-Kettering Cancer Center | U133 A & B | GSE3218 |
| Normal organs/tissues | Novartis | U133A | GSE1133 |

*Gene Expression Omnibus (GEO) Accession Designation

### Hierarchical Clustering Analysis

One way or two ways hierarchical clustering analyses were conducted by using Cluster (Version 2.11) software, and results were visualized in TreeView (Version 1.60) software, both of which are provided for public use by the laboratory of Michael B. Eisen, Ph.D. of Lawrence Berkeley National Lab and the Department of Molecular and Cellular Biology, Univerisity of California at Berkeley.

Selection of Probe-Sets/Genes to Differentiate Cancers from Normal Tissues

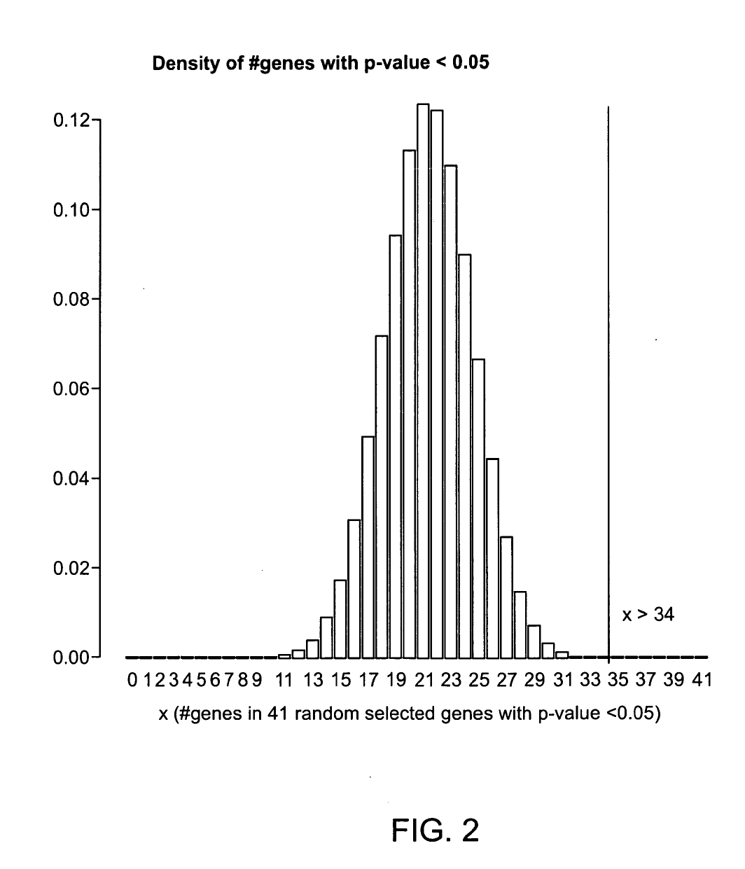

To determine the optimal stringency for selecting probe-sets that can differentiate cancerous from non-cancerous tissues, probe-sets of extreme differential expression between paired HCC and adjacent non-tumorous liver tissue were identified at different selection stringencies ranging from 1 to 16. A stringency of 17 or 18 was not considered because there was only 1 probe set for a stringency of 17 and 0 probe sets for a stringency of 18. These probe-sets were applied to gene expression data for various normal and tumor tissues available in the SCIANTIS™ System Pro microarray database. Data sets for different subtypes of human primary cancers and their corresponding normal tissues were selected for further statistical comparison only if the sets included a minimum of eight samples for both normal and affected cohorts. Data sets for a total of 20 different subtypes of cancers and corresponding normal tissues meeting these criteria were identified. The fraction (q) of total probe-sets (n=22,283) that exhibited a statistically significant difference in expression (p<0.05 by Welch's t-test) between a type of cancer and a normal counterpart according to the data provided in the SCIANTIS™ System Pro database, and the number of highly differentially expressed probe-sets (k), were determined for different selection stringencies. The density distribution [binomial (k,q)] of randomly selected probe-sets from the SCIANTIS™ System Pro database showing significant differences in expression between a specific type of cancer and a corresponding normal tissue was then determined. Using the resulting density distribution curve based on the randomly-selected probe-sets, the statistical significance of k probe-sets to differentiate a cancer from the corresponding normal tissue was determined. FIG. 2 shows an example of such a density distribution, which was constructed using 41 (k) probe-sets, wherein 52.1% (q) of the total probe-sets display a statistically significant difference in expression between breast infiltrating ductal carcinoma and normal breast tissue from the SCIANTIS™ System Pro. In this example, if 34 out of the 41 non-random probe-sets identified by comparison of HCC and adjacent normal tissues show statistically significant differences in expression between infiltrating ductal carcinoma and normal breast tissue based on the data from the SCIANTIS™ System Pro database, the probability of having more than 34 out of 41 randomly selected genes showing statistically significant differential expression between breast cancer and normal breast tissues is very small (p=8.27×10-6). Using this approach, p-values were determined for the probe-sets selected from the study of paired HCC and non-tumorous liver tissue at different stringencies to differentiate different types of cancer and normal tissues in comparison with randomly selected probe-sets. The p-values for all 20 different types of cancer are summarized in FIG. 3. A p-value of “0” means the p-value is less than 1×10⁻¹⁶.

### Validation of Universal Neoplastic Signature Genes

Two-sample Welch t-tests assuming unequal variance between normal and malignant groups were conducted for all 22,238 human probe-sets available on the U133A gene chips for each of 20 subtypes of cancer selected from the SCIANTIS™ System Pro commercial microarray database for this study. The associated t-statistics and p-values were calculated and used to build a distribution curve to assess the likelihood that any 75 randomly selected probe-sets would give smaller p-values than the 75 universal signature probe-sets that were identified in this study. To this end, 10,000 lists of 75 randomly selected probe-sets were generated and each list was applied to each of the 20 different subtypes of cancers. The 1,500 p-values associated with each random list for the 20 subtypes of cancers were sorted and plotted against their ranks. Hierarchical clustering analysis of t-values generated from t-statistics was also employed for validation purposes. Two analyses using 75 probe-sets and 20 different subtypes of cancer and their normal tissues were performed. The seventy five probe-sets identified as universal neoplastic signature in this study were evaluated for the 20 subtypes of cancers and normal tissues. Fifteen hundred t-values were obtained. The 1500 t-values were further analyzed by hierarchical clustering analysis (FIG. 23A). This analysis was repeated for 75 randomly selected probe-sets for the same 20 different sub types of cancers and normal tissues (FIG. 23B).

### Statistical Analyses

Statistical analyses, including Chi-square test, Fisher's exact test, t-test, and survival analyses (log-rank and Wilcoxon tests), were conducted using SAS software (Version 9.1.3).

### Real-Time Quantitative Reverse-Transcriptase Polymerase Chain Reaction (RT-PCR)

TaqMan™ real-time quantitative reverse transcriptase-PCR(qRT-PCR) was used to quantify mRNA. cDNA was synthesized from 8 μg of total RNA for each sample using 1500 ng oligo(dT) primer and 600 units SuperScript™ II Reverse Transcriptase from Invitrogen (Carlsbad, Calif.) in a final volume of 60 μl according to the manufacturer's instructions. For each RT-PCR reaction, 0.5 μl cDNA was used as template in a final volume of 25 μl following the manufacturers' instructions (ABI and Roche). The PCR reactions were carried out using an Applied Biosystems 7900HT Real-Time PCR system. Probes and reagents required for the experiments were obtained from Applied Biosystems (ABI) (Foster City, Calif.). The sequences of primers and the probes used for real-time quantitative RT-PCR are listed in Table 4. Hypoxanthine-guanine phosphoribosyltransferase (HPRT) housekeeping gene was used as an endogenous reference for normalization. All samples were run in duplicate on the same PCR plate for the same target mRNA and the endogenous reference HPRT mRNA. The relative quantities of target mRNAs were calculated by comparative Ct method according to manufacturer's instructions (User Bulletin #2, ABI Prism 7700 Sequence Detection System). A non-tumorous liver sample was chosen as the relative calibrator for calculation.

TABLE 4
Sequences of primers and probes used for real-time quantitative RT-PCR

| Probe | Gene | Probe | Forward | Reverse | |
|---|---|---|---|---|---|
| ID | Symbol | Source | Primer | Primer | Probe |
| 219918_s_at | ASPM | ABI | CAGAAACACCTG | TCCATCACCATTT | CTGGCTTAAGT |
| | | | TAAGGACCAGAA | GAATAGCTTGCA | CTTGAAACTA |
| | | | (SEQ ID NO: 1) | (SEQ ID NO: 41) | (SEQ ID NO: 81) |
| 37425_g_at | CCHCR1 | ABI | GCAGGAGCTAGA | TTGTAACGGGA | TCATGCTGG |
| | | | GAGGGATAAGAA | GAGGAGACCTT | CCACCTTG |
| | | | (SEQ ID NO: 2) | (SEQ ID NO: 42) | (SEQ ID NO: 82) |
| 202705_at | CCNB2 | ABI | GGCCAAGAATGTG | CAGGAGTTTGC | CTTGATGGC |
| | | | GTGAAAGTAAAT | TGCTTGCATAC | GATGAATTT |
| | | | (SEQ ID NO: 3) | (SEQ ID NO: 43) | (SEQ ID NO: 83) |
| 206680_at | CD5L | ABI | TGAAGACACGT | GCCCAGAGCA | CAAGTCAAAG |
| | | | GGGTCGAATG | GAGGTTGTC | GGATCTTCA |
| | | | (SEQ ID NO: 4) | (SEQ ID NO: 44) | (SEQ ID NO: 84) |
| 203213_at | CDC2 | ABI | GCTGAACTAGCAAC | CTTCTGGCCACA | CAAAGCTC |
| | | | TAAGAAACCACTT | CTTCATTATTGG | TGAAAATC |
| | | | (SEQ ID NO: 5) | (SEQ ID NO: 45) | (SEQ ID NO: 85) |
| 209714_s_at | CDKN3 | ABI | CAGCCTGCGA | CAGCTAATTTGTC | CAGACCATCA |
| | | | GACCTAAGAG | CCGAAACTCATG | AGCAATACA |
| | | | (SEQ ID NO: 6) | (SEQ ID NO: 46) | (SEQ ID NO: 86) |
| 205984_at | CRHBP | ABI | GGGACACGTAAA | CAGCTCCACAAA | CTGCTGAGG |
| | | | TGGTCTTCAGTT | GTCTCCTATTCC | ATTTCTTT |
| | | | (SEQ ID NO: 7) | (SEQ ID NO: 47) | (SEQ ID NO: 87) |
| 218002_s_at | CXCL14 | ABI | CGCACTGCGA | GCTCCTGACC | TTGGTGGTG |
| | | | GGAGAAGAT | TCGGTACCT | ATGATAACC |
| | | | (SEQ ID NO: 8) | (SEQ ID NO: 48) | (SEQ ID NO: 88) |
| 220295_x_at | DEPDC1 | ABI | ACTGCAGTGGAAA | TGGCAAAGGAGC | TCCAGGATTTT |
| | | | AACATCTTGACT | AAATAGTCCAT | CAATATGTCCC |
| | | | (SEQ ID NO: 9) | (SEQ ID NO: 49) | (SEQ ID NO: 89) |
| 208394_x_at | ESM1 | ABI | GCAAGTCATCTTC | CATGCCTCAGATG | CTTGAGGAAAGAA |
| | | | CCTACCCATATT | TTTGAAAACCTT | ATCTAGTATTAT |
| | | | (SEQ ID NO: 10) | (SEQ ID NO: 50) | (SEQ ID NO: 90) |
| 213706_at | GPD1 | ABI | GGCCTTTGC | GCCCATTCAGCA | CTGCTCAAT |
| | | | GCGTACAG | ACTCTTTCTC | GGACTTTC |
| | | | (SEQ ID NO: 11) | (SEQ ID NO: 51) | (SEQ ID NO: 91) |
| 206074_s_at | HMGA1 | ABI | GCCGACCAA | TGGTTTCCTTC | AAGACCCG |
| | | | AGGGAAGCA | CTGGAGTTGTG | GAAAACC |
| | | | (SEQ ID NO: 12) | (SEQ ID NO: 52) | (SEQ ID NO: 92) |
| 207165_at | HMMR | ABI | CAAGCATGTTGTGA | CAAGCTGACA | CAACTCAAAT |
| | | | AGTTGAAAGATGA | GCGGAGTTTT | CGGAAGTATC |
| | | | (SEQ ID NO: 13) | (SEQ ID NO: 53) | (SEQ ID NO: 93) |
| 209709_s_at | HMMR | ABI | CAAGCATGTTGTGA | CAAGCTGACA | CAACTCAAAT |
| | | | AGTTGAAAGATGA | GCGGAGTTTT | CGGAAGTATC |
| | | | (SEQ ID NO: 13) | (SEQ ID NO: 53) | (SEQ ID NO: 93) |
| 203819_s_at | IMP-3 | ABI | GCTGGCAGAGTT | GACAACAACTTCT | TTCATTCAC |
| | | | ATTGGAAAAGGA | GCACTTGACAAA | CGTTTTGCC |
| | | | (SEQ ID NO: 14) | (SEQ ID NO: 54) | (SEQ ID NO: 94) |
| 220116_at | KCNN2 | ABI | GAAACTGAATGAC | GTCTTCACTCCTTT | CAAAGACCC |
| | | | CAAGCAAACACT | CGTTTAAGTCAGA | AGAACATCA |
| | | | (SEQ ID NO: 15) | (SEQ ID NO: 55) | (SEQ ID NO: 95) |
| 212193_s_at | LARP1 | ABI | AGGAGGAAACG | CCAGAACTTCT | CAGTTGGC |
| | | | GTGAAGGACTA | CCAGCCCATA | CAGCTTCA |
| | | | (SEQ ID NO: 16) | (SEQ ID NO: 56) | (SEQ ID NO: 96) |
| 218816_at | LRRC1 | ABI | CCGATTTGTGGAG | GTGGAGTGG | AATGAGACGA |
| | | | GATGAGAAAGAT | CTCGCCTTA | GAACACTTC |
| | | | (SEQ ID NO: 17) | (SEQ ID NO: 57) | (SEQ ID NO: 97) |
| 209035_at | MDK | ABI | CCCTGCAACT | CGCACCCCAG | TTTGGAGCC |
| | | | GGAAGAAGGA | TTCTCAAAC | GACTGCAAG |
| | | | (SEQ ID NO: 18) | (SEQ ID NO: 58) | (SEQ ID NO: 98) |
| 204825_at | MELK | ABI | AGGAAGGGTT | TCTGGATTCACTAAT | CCAGAAGACT |
| | | | CTGCCAGAGA | CTAGTTGTAGTCACA | AAAGCTTCAC |
| | | | (SEQ ID NO: 19) | (SEQ ID NO: 59) | (SEQ ID NO: 99) |
| 206797_at | NAT2 | ABI | GCATTCAGCCT | GCCAATTCTTTCAAA | CTGGCCAA |
| | | | AGTTCCTGGTT | ATATGCTTCAATGTC | AGGGATCA |
| | | | (SEQ ID NO: 20) | (SEQ ID NO: 60) | (SEQ ID NO: 100) |
| 204641_at | NEK2 | ABI | AGCGAGCTCT | CTAGTCTCTCAC | ATTGGAGCA |
| | | | CAAAGCAAGA | GAACACAAAGCT | GAAAGAACA |
| | | | (SEQ ID NO: 21) | (SEQ ID NO: 61) | (SEQ ID NO: 101) |
| 221529_s_at | PLVAP | ABI | CCTGCAGGC | CGGGCCAT | CCCCATCC |
| | | | ATCCCTGTA | CCCTTGGT | AGTGGCTG |
| | | | (SEQ ID NO: 22) | (SEQ ID NO: 62) | (SEQ ID NO: 102) |
| 218009_s_at | PRC1 | ABI | CCGTCCCTCT | GTAGCATCAGATT | CTTCAGCG |
| | | | CTGACAGTTC | TGGAAGCCTTTG | AGAACTTT |
| | | | (SEQ ID NO: 23) | (SEQ ID NO: 63) | (SEQ ID NO: 103) |
| 203554_x_at | PTTG1 | ABI | CCTCAGATGATGC | CTCTTCAGGCAG | CTTCAATCCT |
| | | | CTATCCAGAAAT | GTCAAAACTCT | CTAGACTTTG |
| | | | (SEQ ID NO: 24) | (SEQ ID NO: 64) | (SEQ ID NO: 104) |
| 207714_s_at | SERPINH1 | ABI | CGTGGGTGTCA | TCCTTCTCGTCG | CCGGACAG |
| | | | TGATGATGCA | TCGTAGTAGTT | GCCTCTAC |
| | | | (SEQ ID NO: 25) | (SEQ ID NO: 65) | (SEQ ID NO: 105) |
| 217714_x_at | STMN1 | ABI | CAAATGGCTGC | GTTCTTCCGCAC | TTTGCGAGAG |
| | | | CAAACTGGAA | TTCTTCAATGTG | AAGGATAAG |
| | | | (SEQ ID NO: 26) | (SEQ ID NO: 66) | (SEQ ID NO: 106) |
| 210609_s_at | TP5313 | ABI | CGCCTTCCAGC | CCTGCATGGATT | CAGCCTGAAC |
| | | | TGTTACATCTT | AGCACATAGTCT | ATTTCCCAC |
| | | | (SEQ ID NO: 27) | (SEQ ID NO: 67) | (SEQ ID NO: 107) |
| 204822_at | TTK | ABI | TGGCTCATCCCTA | CCAGTTAAC | |
| | | | TGTTCAAATTCA | CAAATGGCC | |
| | | | (SEQ ID NO: 28) | (SEQ ID NO: 68) | |
| 205019_s_at | VIPR1 | ABI | GCTATCCTCTACT | CAGCGCCG | CCGCCTGC |
| | | | GCTTCCTCAATG | CCACTTC | ACCTCAC |
| | | | (SEQ ID NO: 29) | (SEQ ID NO: 69) | (SEQ ID NO: 108) |
| HPRT 1 | HPRT 1 | ABI | Not provided | | |
| | | | by the | | |
| | | | manufacturer | | |
| 202715_at | CAD | Roche | CCCGTGTCAA | CAGAGCCAT | CCAGGCTG |
| | | | CGAGATAAGC | GCGGATGTA | (SEQ ID NO: 109) |
| | | | (SEQ ID NO: 30) | (SEQ ID NO: 70) | |
| 205392_s_at | CCL14/// | Roche | AGCTTCCCAC | GTGGTAAGGT | CTTCCTCC |
| | CCL15 | | AGCATGAAGA | CCCCGTGAG | (SEQ ID NO: 110) |
| | | | (SEQ ID NO: 31) | (SEQ ID NO: 71) | |
| 207828_s_at | CENPF | Roche | GAGTCCTCCA | TCCGCTGAGC | GGCAGCAG |
| | | | AACCAACAGC | AACTTTGAC | (SEQ ID NO: 111) |
| | | | (SEQ ID NO: 32) | (SEQ ID NO: 72) | |
| 211981_at | COL4A1 | Roche | AGAGGAGCGAG | TCAGGCTTCATT | GAAGGCAG |
| | | | ATGTTCAAGA | ATGTTCTTCTCA | (SEQ ID NO: 112) |
| | | | (SEQ ID NO: 33) | (SEQ ID NO: 73) | |
| 205866_at | FCN3 | Roche | CCTCGGTGAG | CTGTGGAGGC | CTGGGCAA |
| | | | GTAGACCACT | TCAGGGAAT | (SEQ ID NO: 113) |
| | | | (SEQ ID NO: 34) | (SEQ ID NO: 74) | |
| 218663_at | HCAP-G | Roche | TTTAGAACTCAG | AGCTCTCAGACA | TCTGGAGC |
| | | | TAGCCATCTTGC | TGTCCTATCTTT | (SEQ ID NO: 114) |
| | | | (SEQ ID NO: 35) | (SEQ ID NO: 75) | |
| 219494_at | RAD54B | Roche | TCATGATCTGC | TTTTTCCAACG | CAGGAGAA |
| | | | TTGACTGTGAG | AATCACCTGT | (SEQ ID NO: 115) |
| | | | (SEQ ID NO: 36) | (SEQ ID NO: 76) | |
| 209219_at | RDBP | Roche | ATGCTGGAT | CCCTTAGGGC | CTGGGGCT |
| | | | GCCGCTACT | TGTTCTGGA | (SEQ ID NO: 116) |
| | | | (SEQ ID NO: 37) | (SEQ ID NO: 77) | |
| 201342_at | SNRPC | Roche | AGGAAAGATACCT | ATACCAGGG | CTCCTCCT |
| | | | CCTACTCCATTC | CGAGGAGGA | (SEQ ID NO: 117) |
| | | | (SEQ ID NO: 38) | (SEQ ID NO: 78) | |
| 201291_s_at | TOP2A | Roche | TTGTGGAAAGA | CATCTTGTTTT | GGAGGCTG |
| | | | AGACTTGGCTA | TCCTTGGCTTC | (SEQ ID NO: 118) |
| | | | (SEQ ID NO: 39) | (SEQ ID NO: 79) | |
| 201292_at | TOP2A | Roche | TTGTGGAAAGA | CATCTTGTTTT | GGAGGCTG |
| | | | AGACTTGGCTA | TCCTTGGCTTC | (SEQ ID NO: 118) |
| | | | (SEQ ID NO: 39) | (SEQ ID NO: 79) | |
| HPRT 1 | HPRT 1 | Roche | TGATAGATCCATTC | AAGACATTCTTTCC | TGGTGGAG |
| | | | CTATGACTGTAGA | AGTTAAAGTTGAG | (SEQ ID NO: 119) |
| | | | (SEQ ID NO: 40) | (SEQ ID NO: 80) | |

### Results

In order to identify tumor specific-genes that are specifically expressed in hepatocellular carcinoma tissues, gene expression profiles were generated for 18 pairs of HCC and adjacent non-tumorous liver tissue samples as described above. To ensure that the profiles included genes with robust expression, only those genes showing significant differential expression by both MAS 5.0 and dChip software were selected. The number of probe sets corresponding to genes showing significant differential expression between hepatocellular carcinoma and adjacent non-tumorous liver tissues in 18 paired samples using different selection stringencies are shown in Table 5. The number of probe-sets showing significant differential expression increased as the stringency was relaxed (i.e., from genes differentially expressed between HCC and normal tissues in all 18 sample pairs (high selection stringency of 18) to genes differentially expressed between HCC and normal tissues in 1 out of 18 sample pairs (low selection stringency of 1).

TABLE 5
Number of highly differentially expressed
genes at different stringencies.

| | Number of probe | | | Number of probe sets | | |
|---|---|---|---|---|---|---|
| | sets judged as | | | judged as “present” in | | |
| | “present” in | | | non-tumorous liver | | |
| | tissue of hepatocellular | | | tissues and “absent | | |
| | carcinoma and | | | or marginal” in | | |
| | “absent or marginal” | | | paired tissue | | |
| | in paired | | | of hepatocellular | | |
| | non-tumorous liver tissues | | | carcinoma | | |
| Selection | MAS | | | MAS | | |
| Stringency* | 5.0 | dChip | Both | 5.0 | dChip | Both |
| 18 (100%) | 4 | 1 | 0 | 0 | 0 | 0 |
| 17 (94%) | 10 | 4 | 1 | 0 | 1 | 0 |
| 16 (89%) | 14 | 12 | 2 | 2 | 2 | 1 |
| 15 (83%) | 40 | 22 | 8 | 7 | 6 | 3 |
| 14 (78%) | 75 | 50 | 15 | 13 | 13 | 3 |
| 13 (72%) | 130 | 95 | 32 | 28 | 22 | 9 |
| 12 (67%) | 232 | 160 | 59 | 43 | 33 | 16 |
| 11 (61%) | 392 | 269 | 94 | 65 | 58 | 29 |
| 10 (56%) | 587 | 458 | 142 | 119 | 95 | 44 |
| 9 (50%) | 919 | 733 | 253 | 201 | 174 | 71 |
| 8 (44%) | 1358 | 1184 | 439 | 310 | 290 | 110 |
| 7 (39%) | 1918 | 1747 | 725 | 490 | 492 | 175 |
| 6 (33%) | 2589 | 2522 | 1135 | 756 | 879 | 298 |
| 5 (28%) | 3444 | 3501 | 1705 | 1149 | 1500 | 499 |
| 4 (22%) | 4432 | 4717 | 2520 | 1771 | 2436 | 882 |
| 3 (17%) | 5623 | 6167 | 3633 | 2743 | 3729 | 1474 |
| 2 (11%) | 7059 | 7924 | 5105 | 4194 | 5628 | 2595 |
| 1 (6%) | 9309 | 10291 | 7558 | 6676 | 8609 | 4855 |
| 0 (0%) | 22283 | 22283 | 22283 | 22283 | 22283 | 22283 |

*Selection stringency is defined in page 13, lines 16-24.

To determine the optimal stringency for selecting probe-sets that can differentiate cancerous from non-cancerous tissues, different selection stringencies were applied to gene expression data sets for various normal and tumor tissues available in the SCIANTIS™ System Pro microarray database. Data sets for different subtypes of human primary cancers and their corresponding normal tissues were selected if the sets included a minimum of eight samples for both normal and affected cohorts. Data sets for a total of 20 different subtypes of cancers and corresponding normal tissues meeting these criteria were identified (Table 6).

TABLE 6
Numbers of samples in the SCIANTIS ™ System Pro Database for 20
different types of cancer and corresponding normal tissues used in the present study.

| | Sample | | Sample |
|---|---|---|---|
| Type of Cancer | No. | Normal Tissue | No. |
| Breast, Infiltrating Ductal Carcinoma, | 169 | Breast, Normal | 68 |
| Primary | | | |
| Breast, Infiltrating Lobular Carcinoma, | 17 | Breast, Normal | 68 |
| Primary | | | |
| Colon, Adenocarcinoma (Excluding | 77 | Colon, Normal | 180 |
| Mucinous Type), Primary | | | |
| Colon, Adenocarcinoma, Mucinous Type, | 7 | Colon, Normal | 180 |
| Primary | | | |
| Endometrium, Adenocarcinoma, | 50 | Endometrium, Normal | 23 |
| Endometrioid Type, Primary | | | |
| Kidney, Renal Cell Carcinoma, Clear Cell | 45 | Kidney, Normal | 81 |
| Type, Primary | | | |
| Kidney, Renal Cell Carcinoma, Non-Clear | 15 | Kidney, Normal | 81 |
| Cell Type, Primary | | | |
| Liver, Hepatocellular Carcinoma | 16 | Liver, Normal | 42 |
| Lung, Adenocarcinoma, Primary | 46 | Lung, Normal | 42 |
| Lung, Squamous Cell Carcinoma, Primary | 39 | Lung, Normal | 126 |
| Ovary, Adenocarcinoma, Endometrioid | 22 | Ovary, Normal | 89 |
| Type, Primary | | | |
| Ovary, Adenocarcinoma, Papillary | 36 | Ovary, Normal | 89 |
| SerousType, Primary | | | |
| Pancreas, Adenocarcinoma, Primary | 23 | Pancreas, Normal | 46 |
| Prostate, Adenocarcinoma, Primary | 86 | Prostate, Normal | 57 |
| Rectum, Adenocarcinoma (Excluding | 29 | Rectum, Normal | 44 |
| Mucinous Type), Primary | | | |
| Skin, Malignant Melanoma, Primary | 7 | Skin, Normal | 61 |
| Stomach, Adenocarcinoma (Excluding | 27 | Stomach, Normal | 52 |
| Signet Ring Cell Type), Primary | | | |
| Stomach, Adenocarcinoma, Signet Ring | 9 | Stomach, Normal | 52 |
| Cell Type, Primary | | | |
| Stomach, Gastrointestinal Stromal Tumor | 9 | Stomach, Normal | 52 |
| (GIST), Primary | | | |
| Thyroid Gland, Papillary Carcinoma, | 29 | Thyroid Gland, Normal | 24 |
| Primary; All Variants | | | |

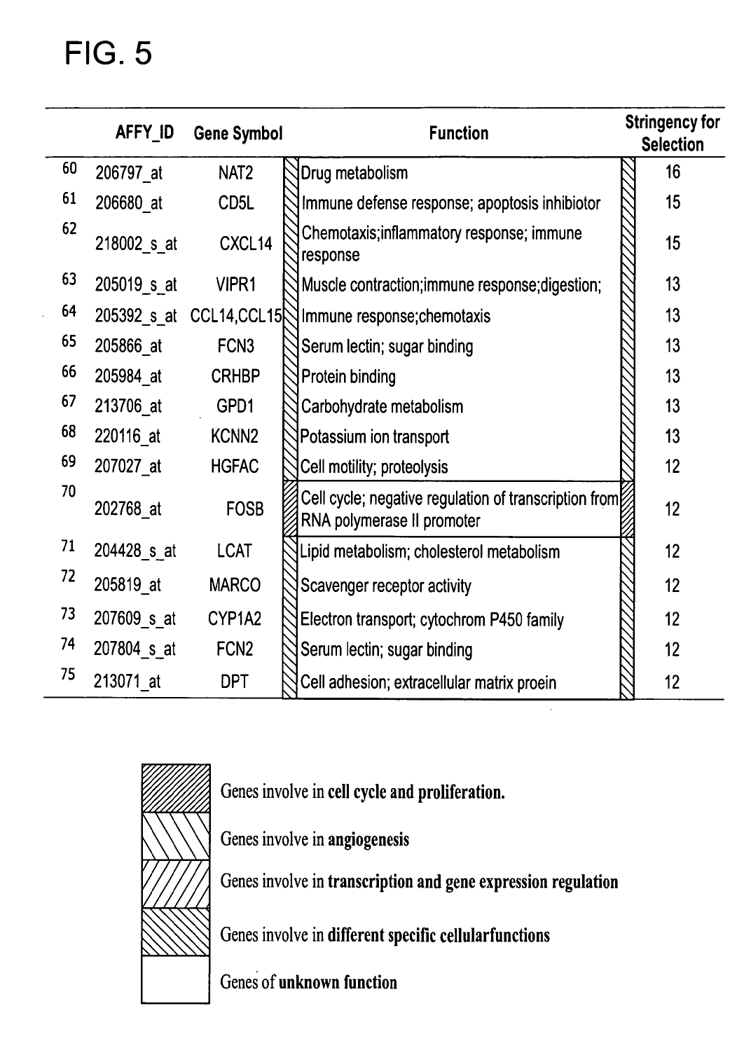

The fraction (q) of total probe-sets (n=22,283) that exhibited a statistically significant difference in expression (p<0.05 by Welch's t-test) between a type of cancer and a normal counterpart according to the data provided in the SCIANTIS™ System Pro database, and the number of highly differentially expressed probe-sets (k), were determined at the 18 different selection stringencies shown in Table 5. This systematic statistical analysis revealed that a stringency of 12 out of 18 pairs selected for 75 probe-sets that could differentiate cancer tissues from their respective normal tissues with p-values <0.005 for 19 out of 20 different cancer subtypes (FIG. 3). The 75 probe-sets selected at this stringency included 59 probe-sets that were specifically expressed in HCC tissues and 16 probe-sets that were specifically expressed in non-tumorous liver tissue. The 75 probe-sets represented a total of 71 different genes because four genes—Top2A, CCHCR1, CDC2 and HMMR—were each represented by two probe sets. These 71 genes and their functions are listed in FIGS. 4 and 5.

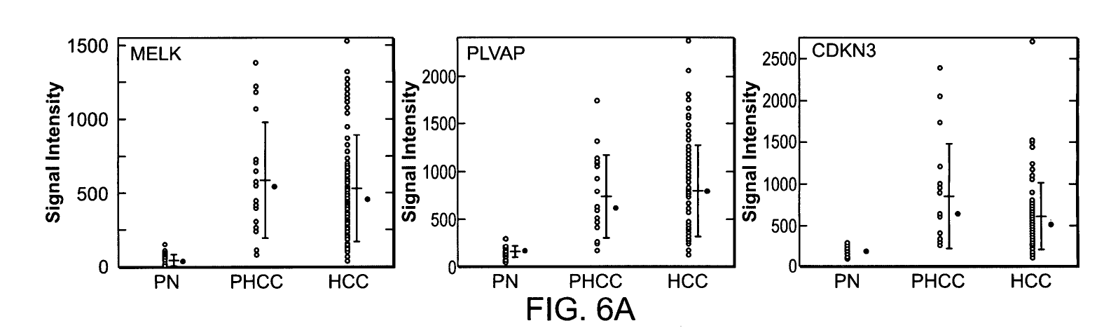

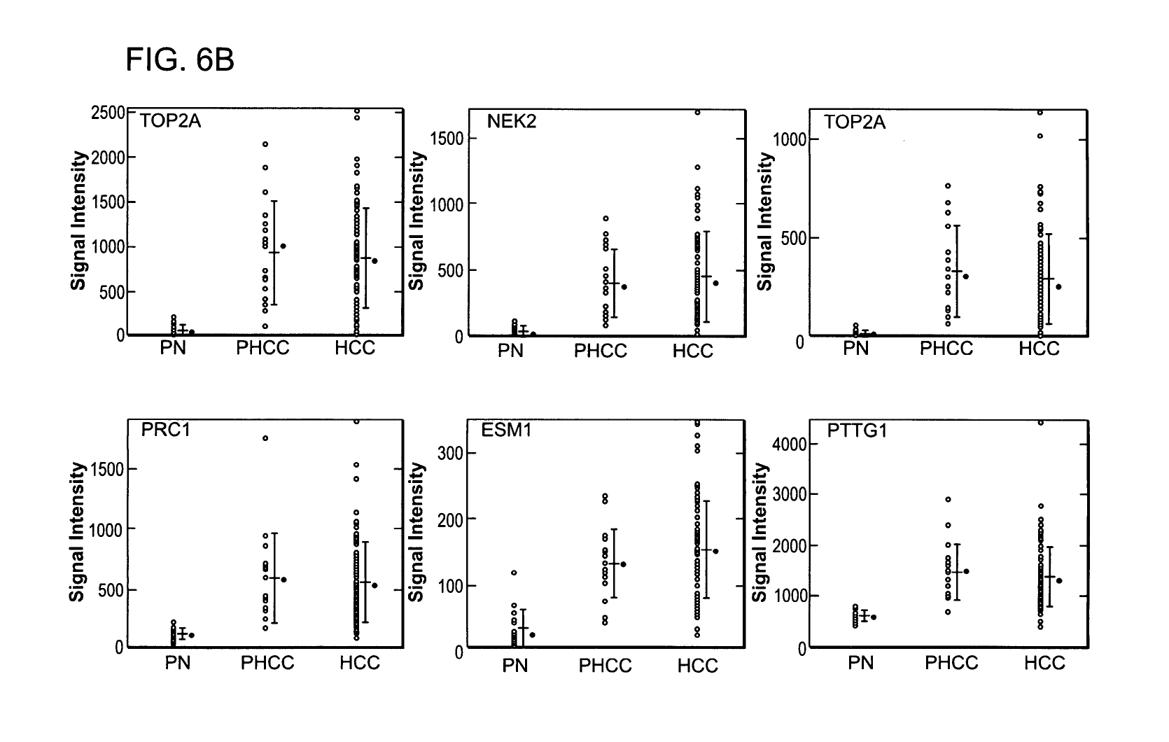

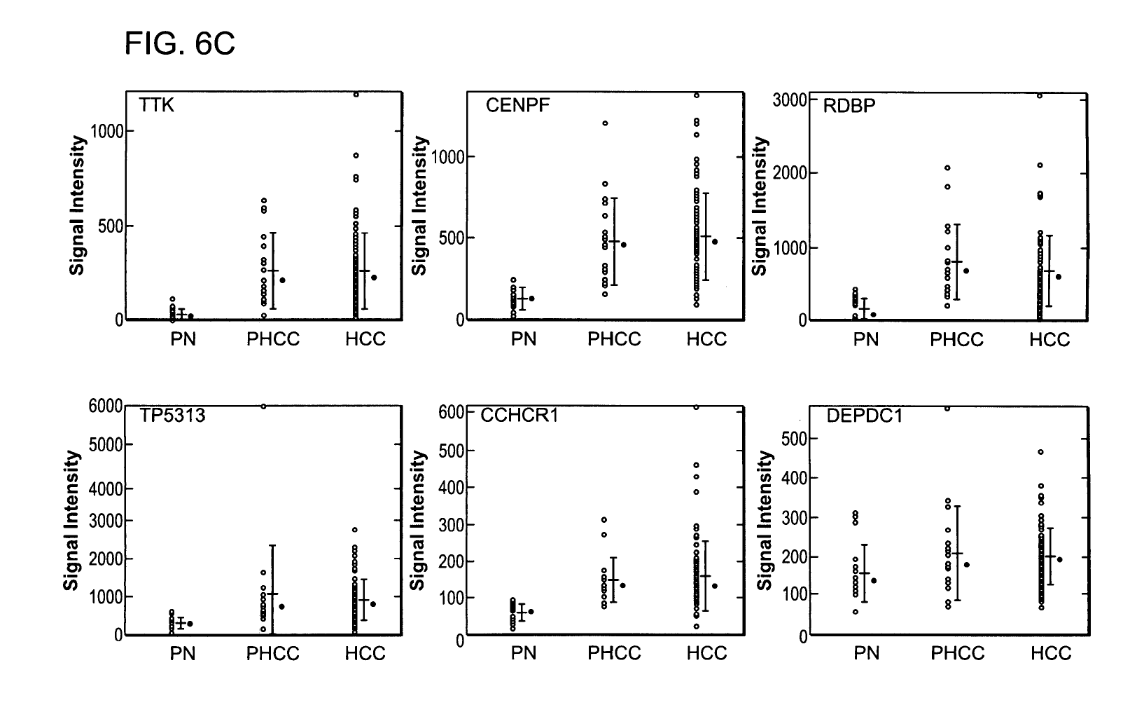

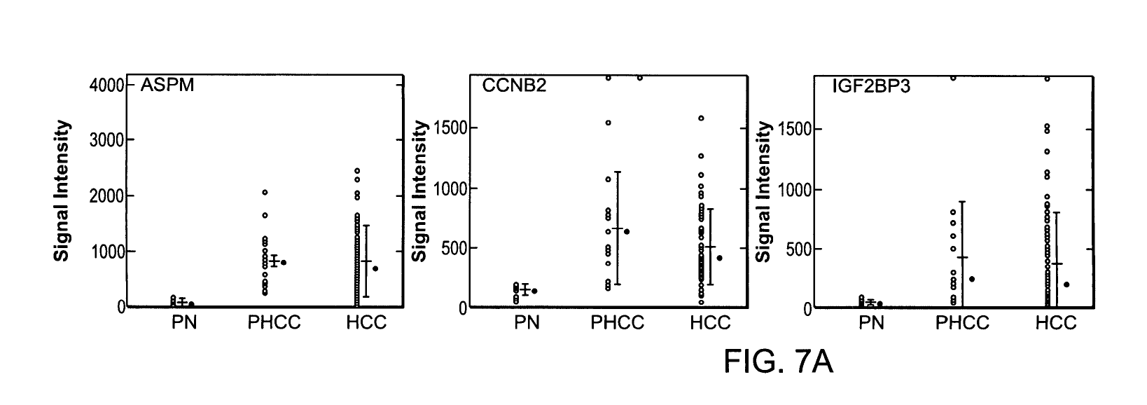

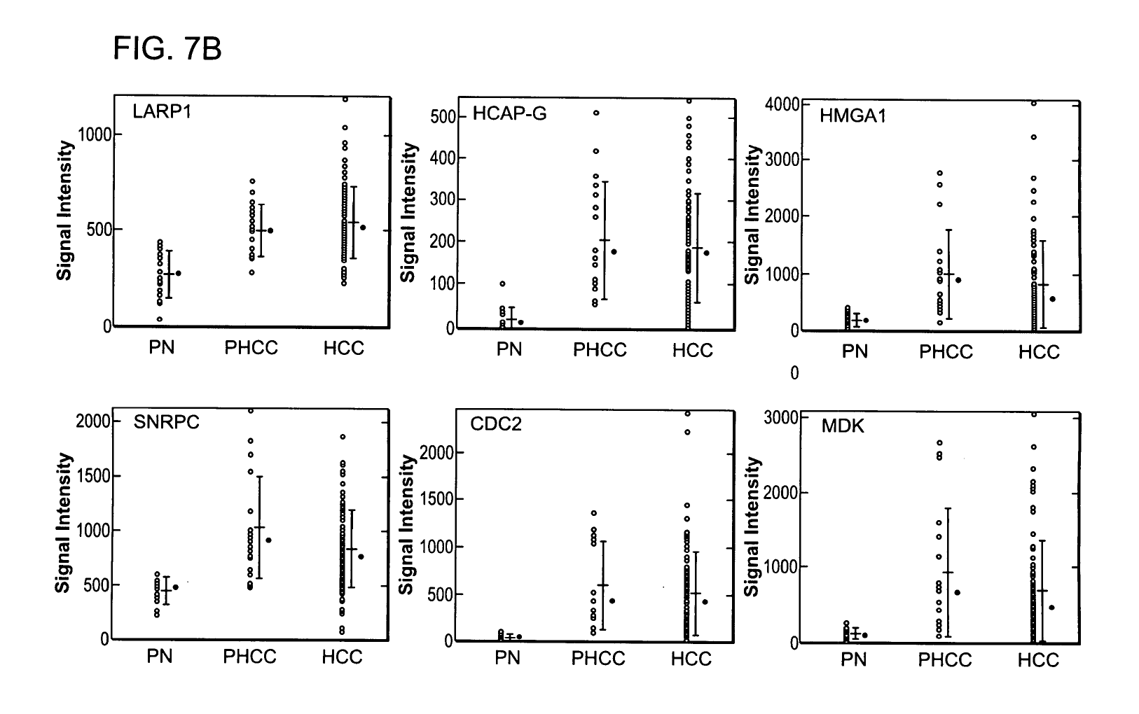

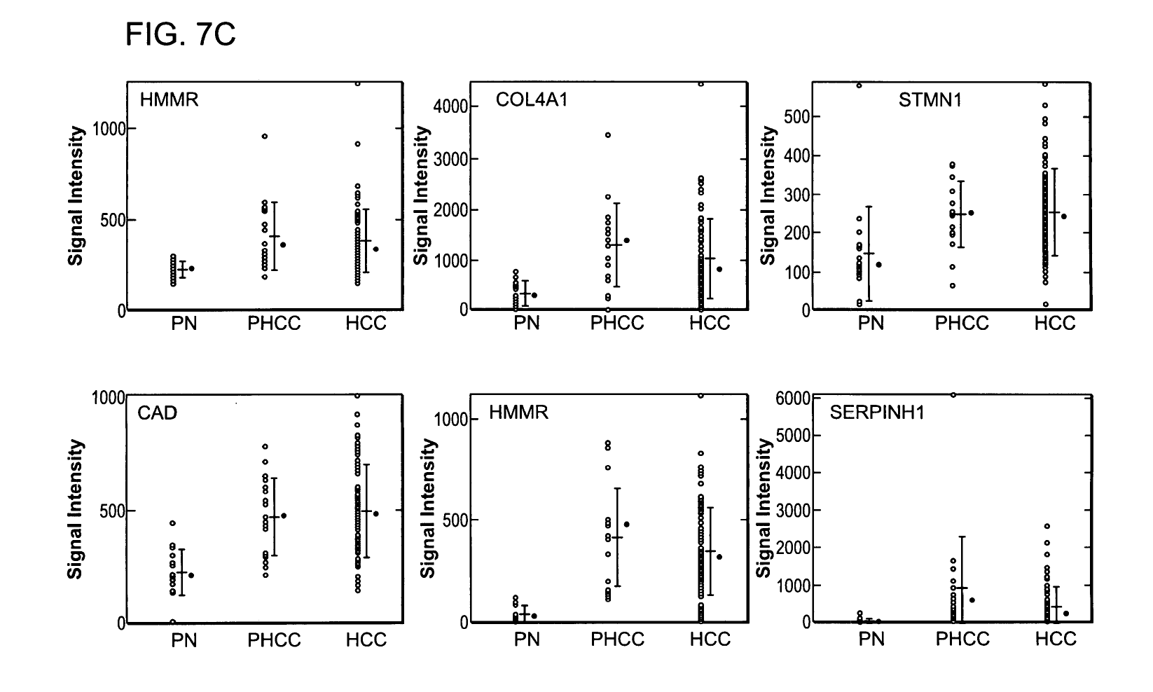

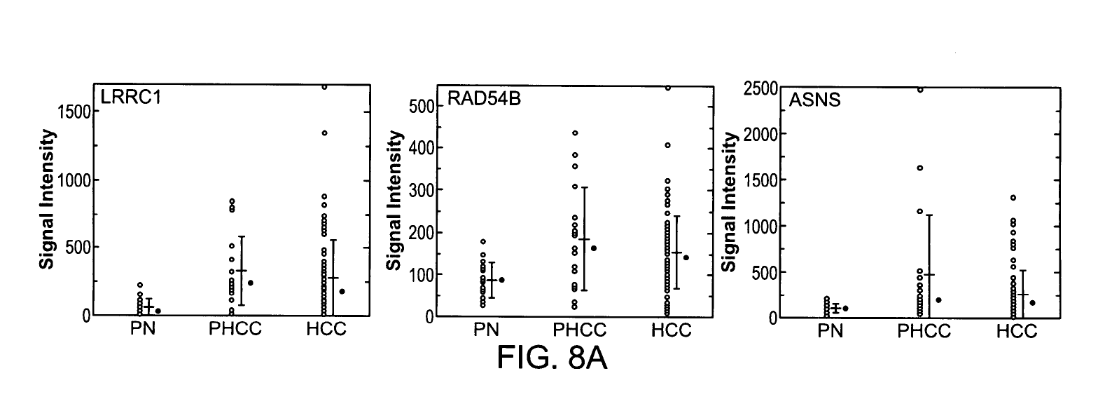

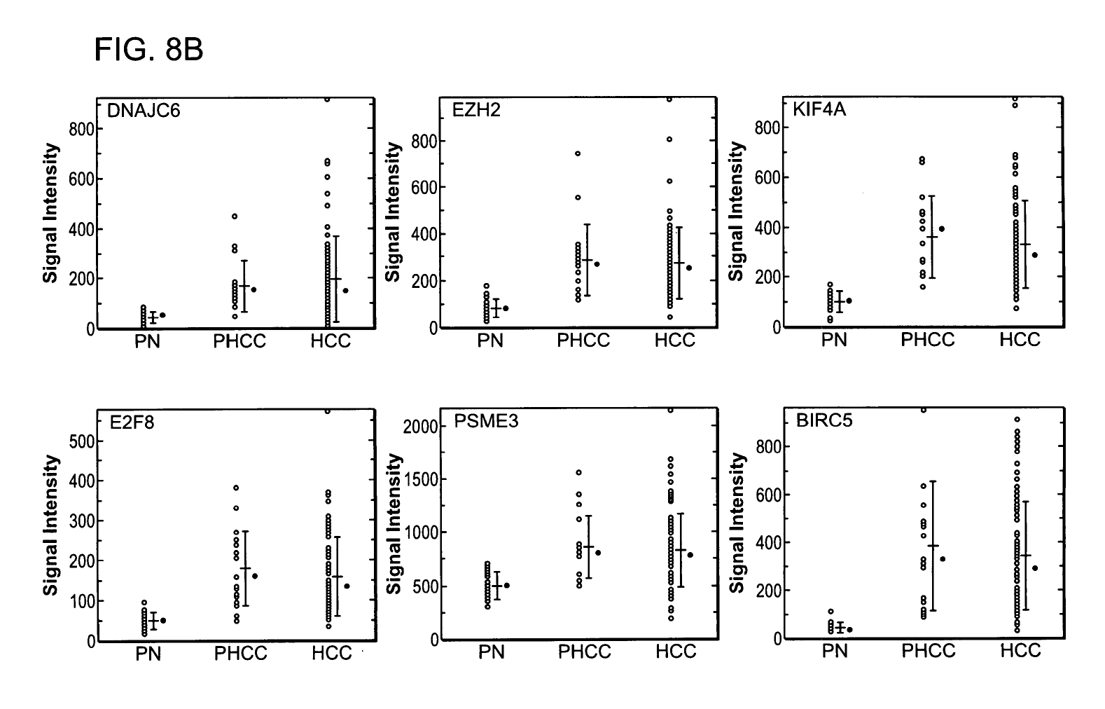

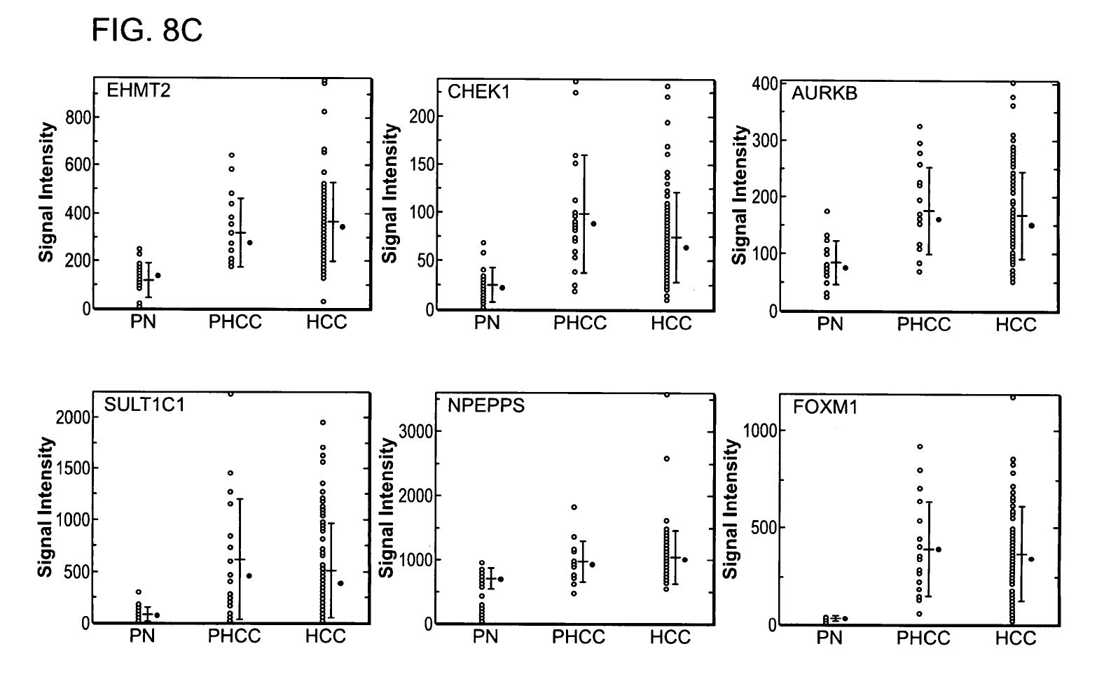

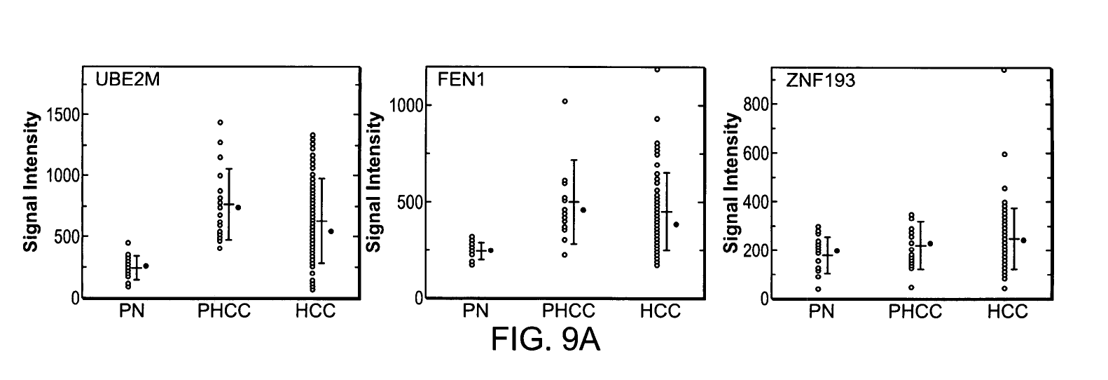

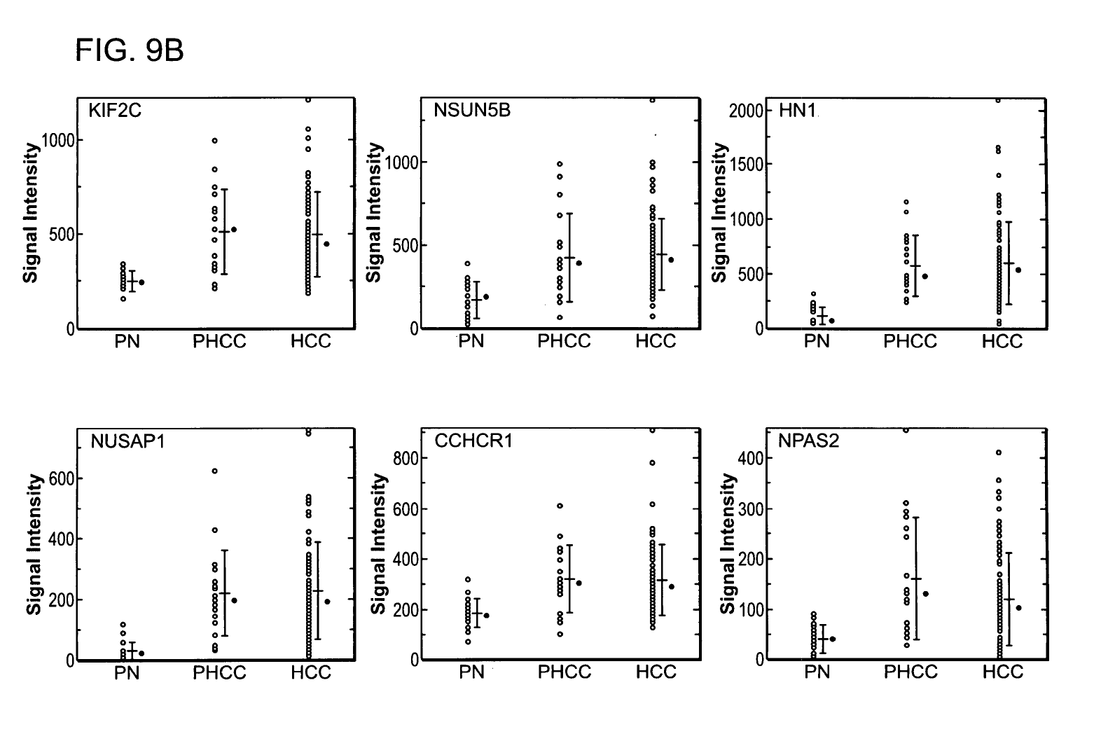

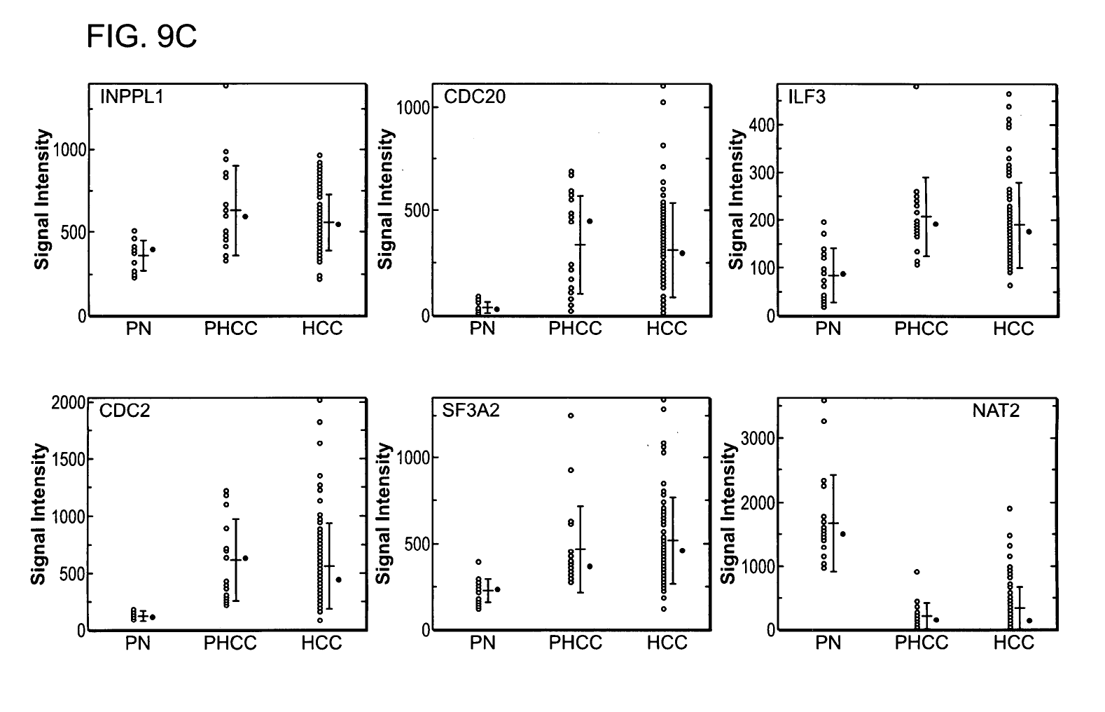

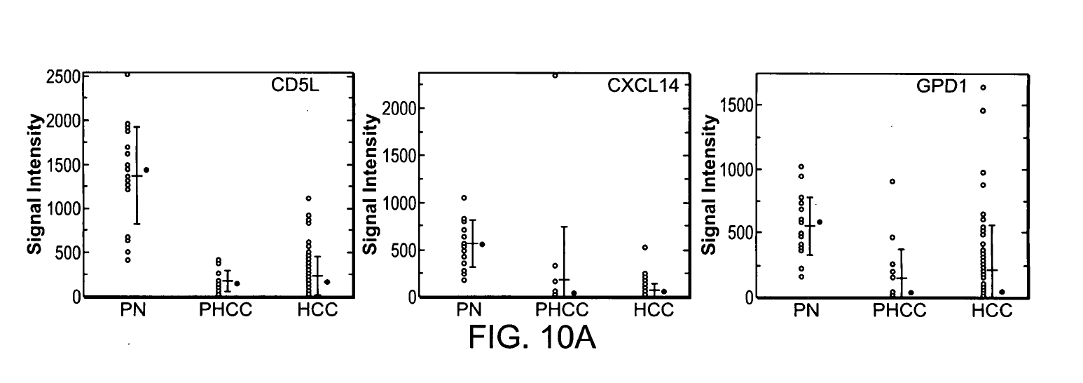

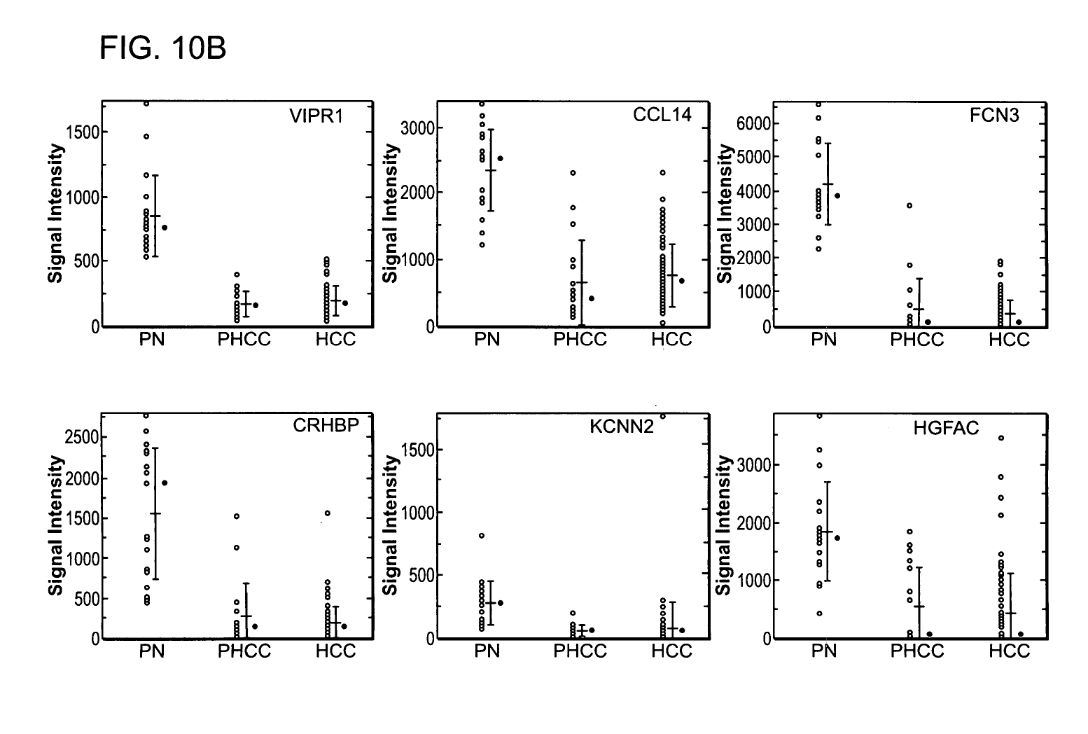

The expression intensities of the genes represented by the 75 probe-sets were compared in the microarray data obtained from HCC and adjacent non-tumorous liver tissues. There was little overlap in expression intensities of these genes between the paired HCC and adjacent non-tumorous liver tissue samples (FIGS. 6-10).

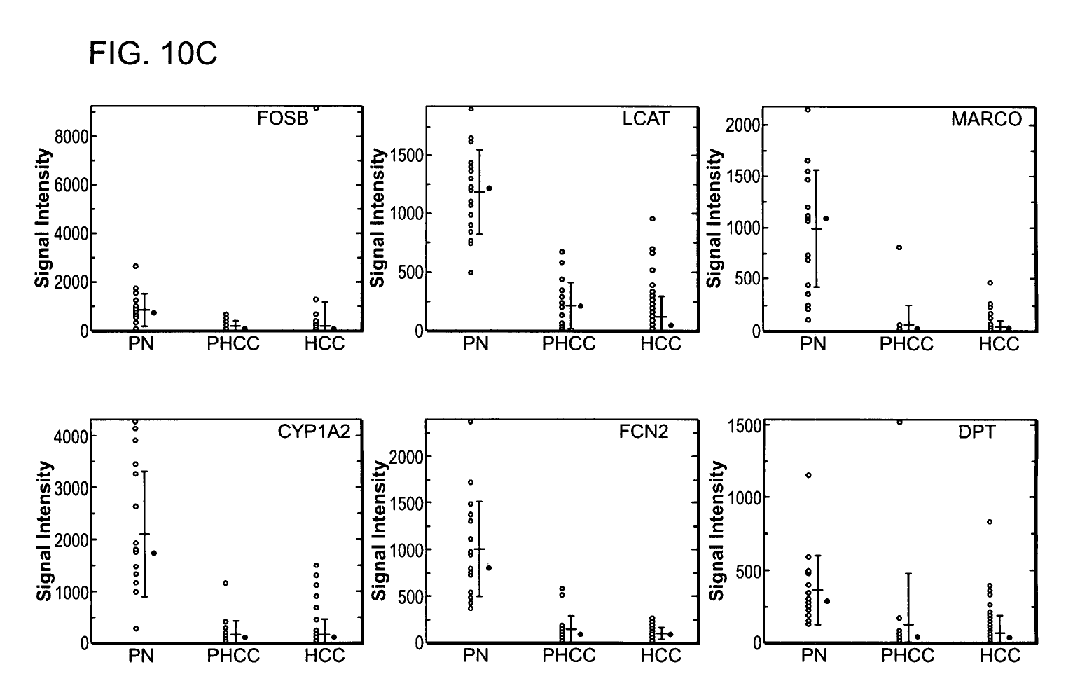

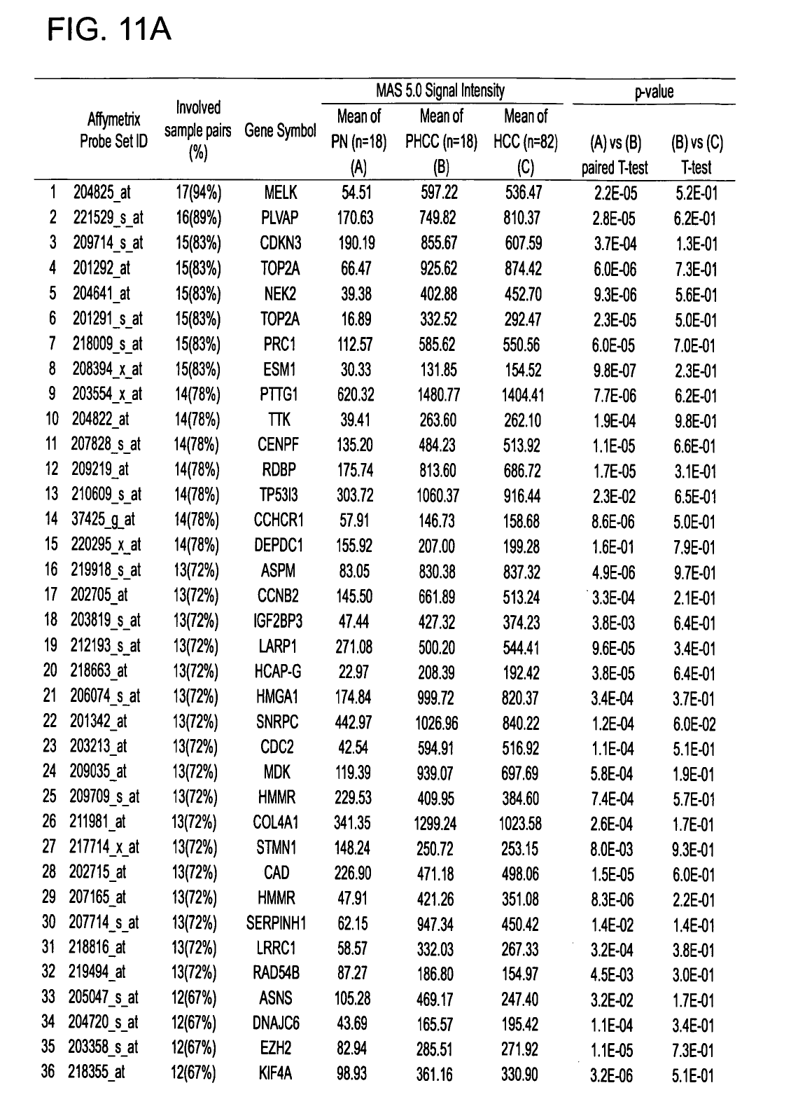

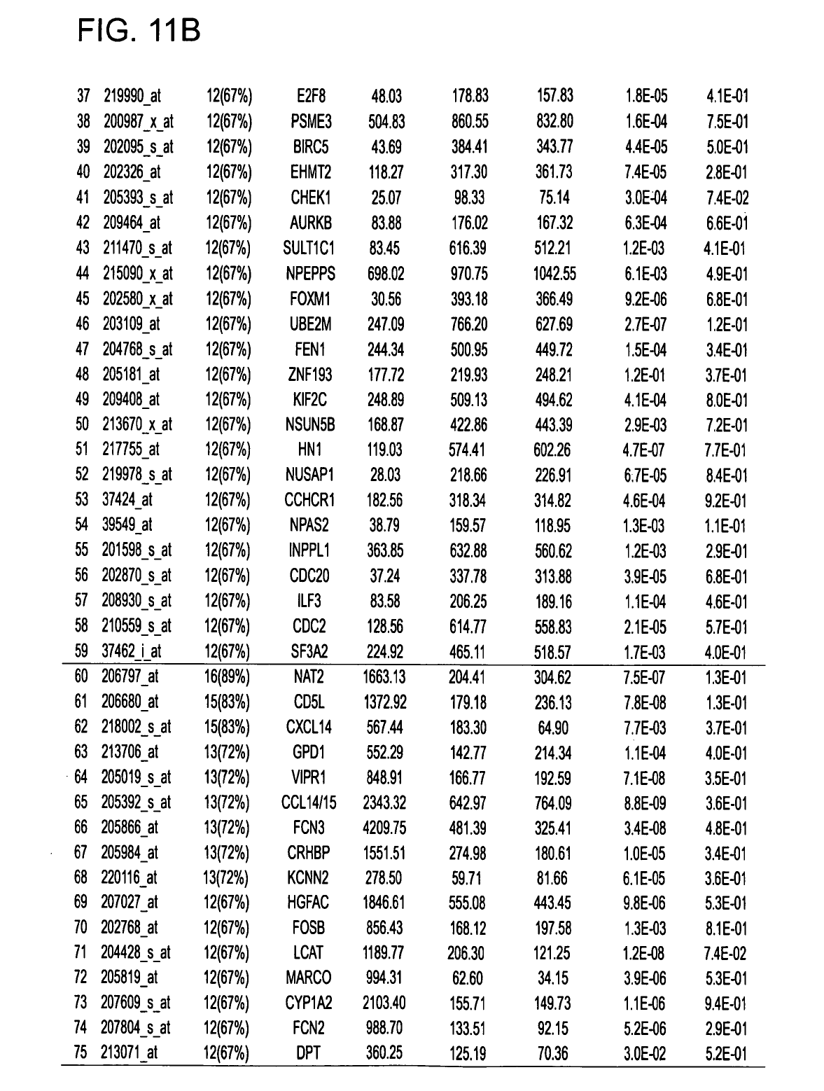

To confirm that the 18 paired HCC samples used in this study were sufficiently representative of this type of cancer, gene expression intensities of the 75 probe-sets were assessed in 82 additional HCC samples, in the absence of paired adjacent non-tumorous liver tissues. As shown in FIGS. 6-10, the gene expression intensities of the 75 probe-sets were similar between the 18 paired HCC samples and the 82 non-paired HCC samples. Statistical comparison of the paired HCC samples and the additional non-paired samples showed no significant difference in the expression of any of the genes in the 75 probes sets, and both groups exhibited similar average expression intensities for each of the 75 probe-sets (FIG. 11).

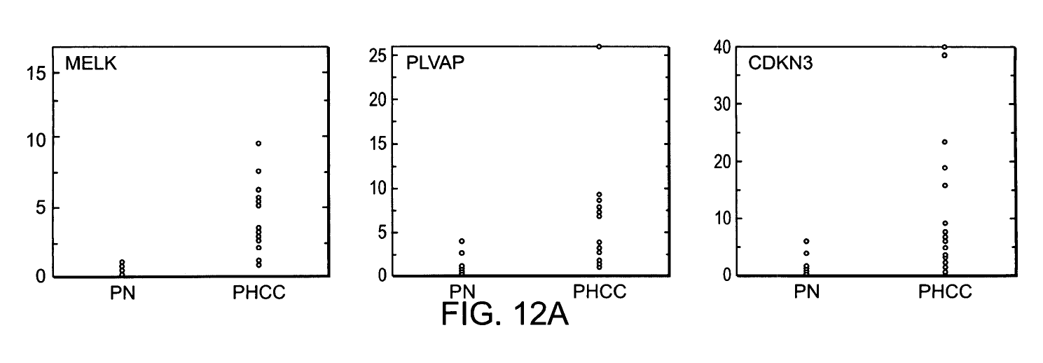

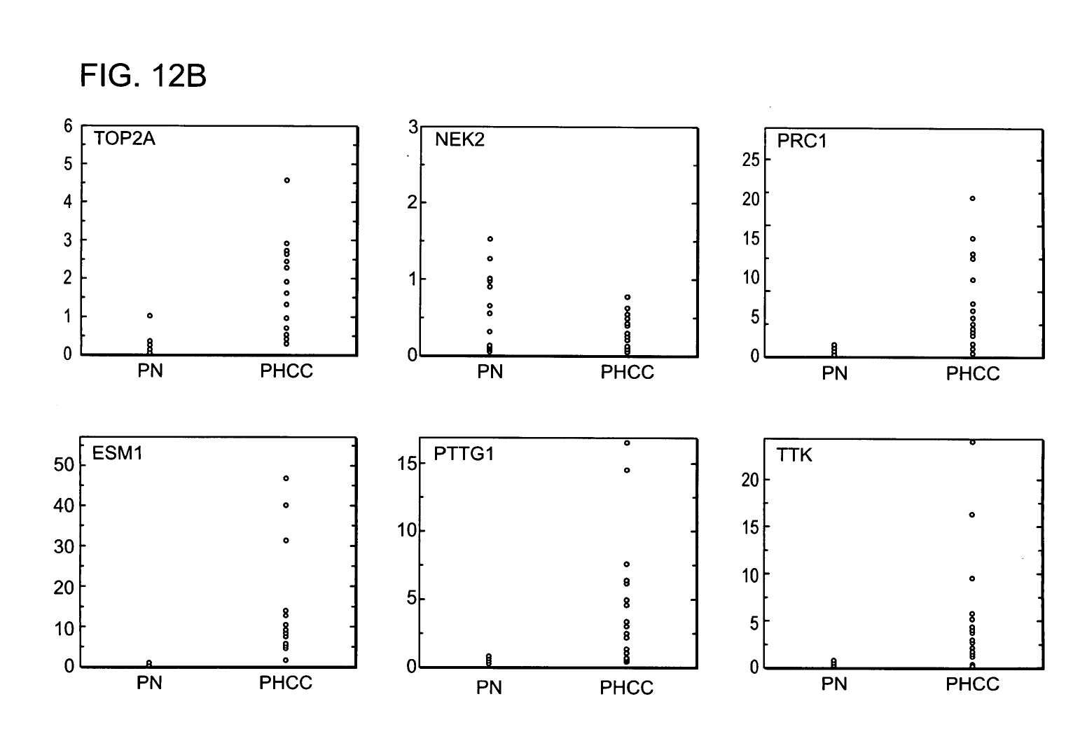

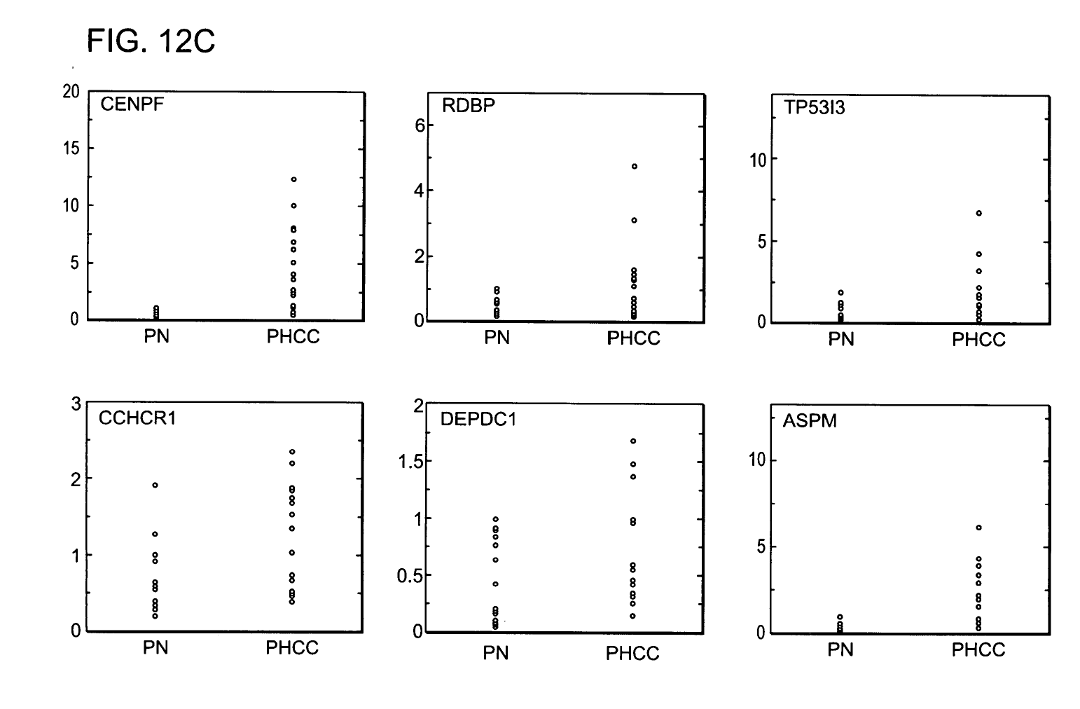

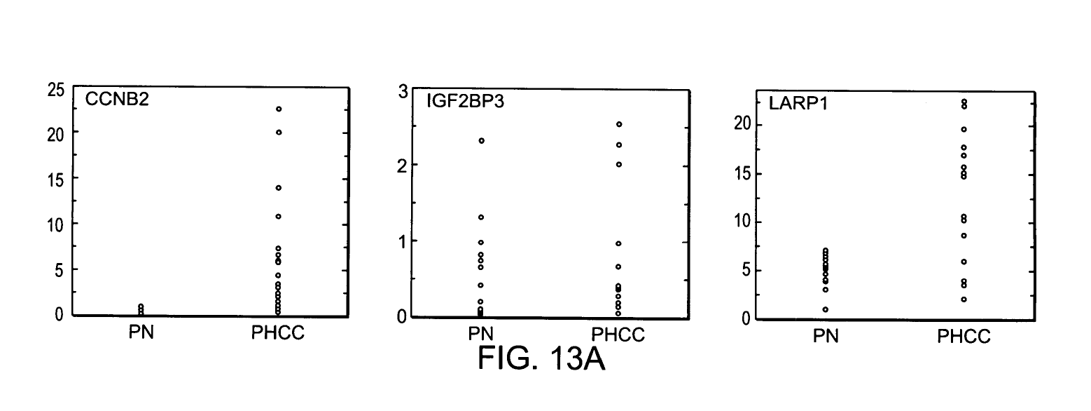

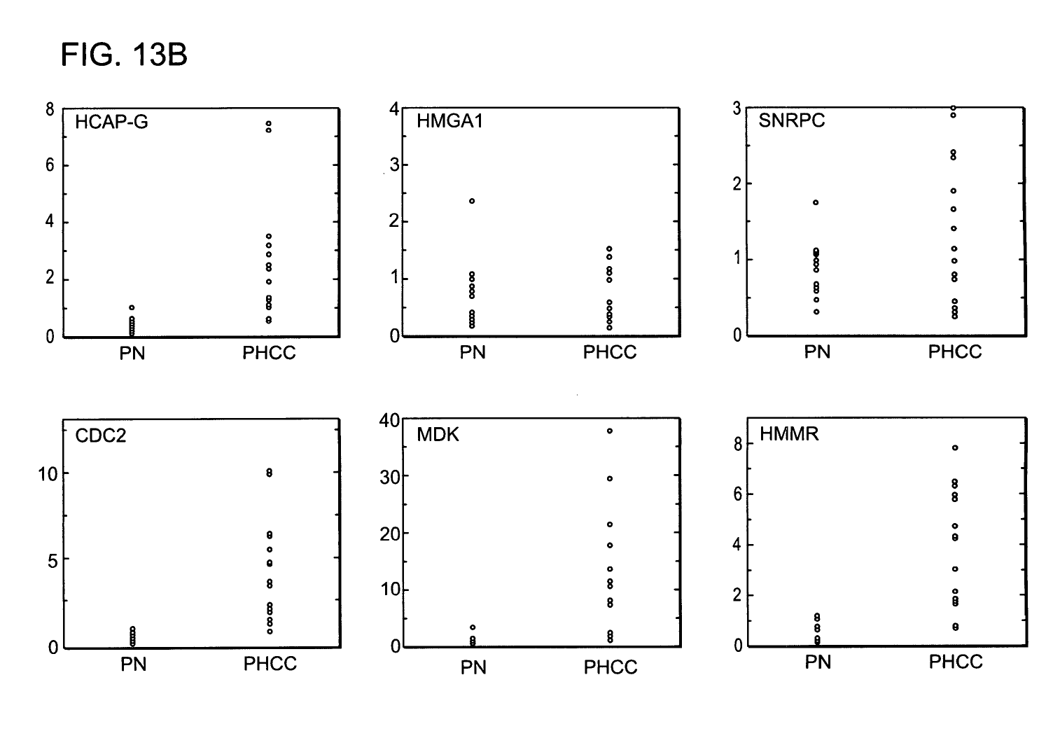

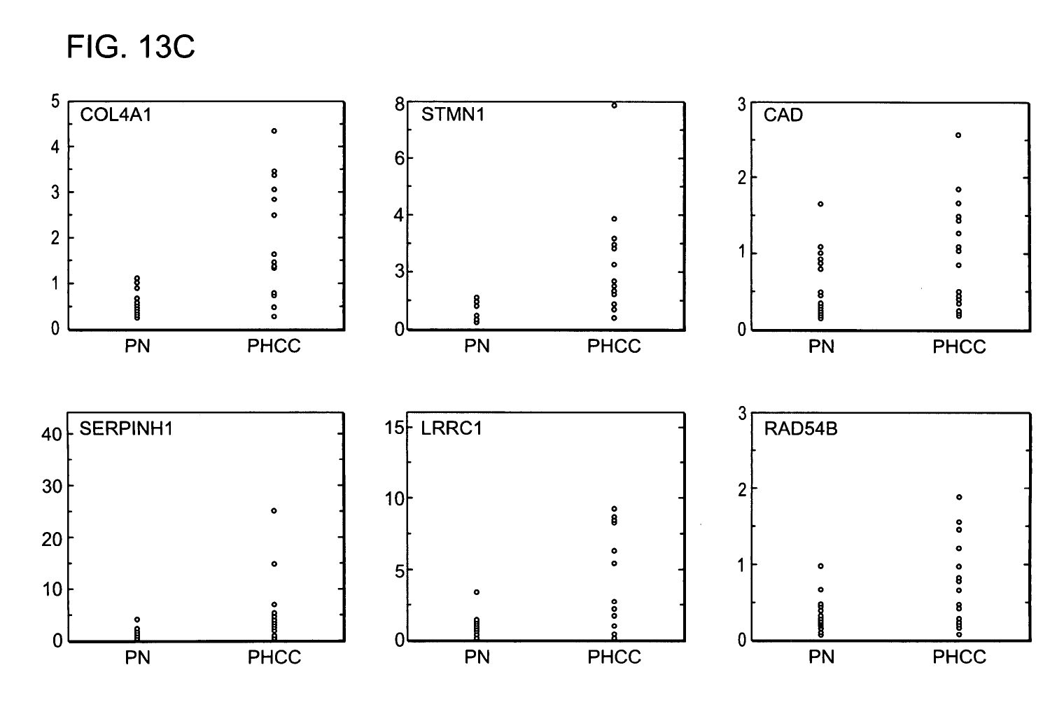

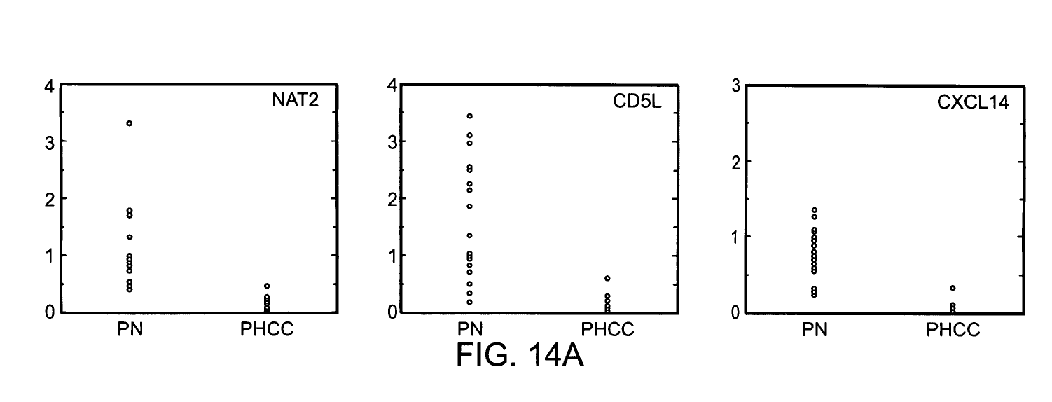

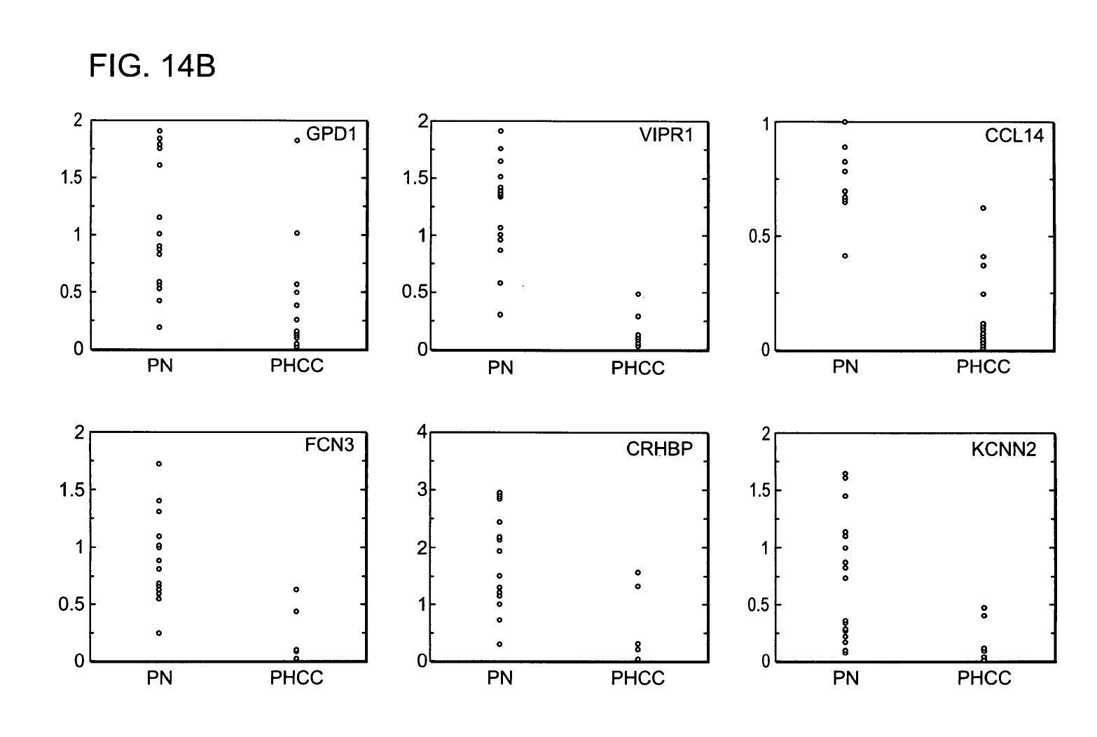

To validate the finding that these 75 probe-sets represented genes displaying significant differential expression between HCC and non-tumorous liver tissues, a series of real-time quantitative reverse transcriptase polymerase chain reaction (RT-qPCR) experiments were conducted on RNA samples from the 18 paired HCC and non-tumorous liver tissues used in the study. The available RNA samples were sufficient to study 39 of the genes represented in the CNS. All 39 genes had appropriate 3′ end DNA sequence across an intron for reliable RT-qPCR study. The results FIGS. 12-14 confirmed that these 39 genes were highly differentially expressed, consistent with the results of the microarray study (FIGS. 6-10).

### Example 2

### Functional Characteristics of the Genes Displaying Significant Differential Expression between Cancer and Normal Tissues

### Materials and Methods

Functional annotation of the significant differential expression genes represented by the 75 probe-sets described in Example 1 was obtained using the Bioinformatic Harvester database of the Karlsruhe Institute of Technology and the Ingenuity Pathway Analysis database (Ingenuity® Systems).

### Results

In the Bioinformatic Harvester database, the 55 genes represented by the 59 tumor-specific probe-sets were designated as having the following biological functions: cell cycle/proliferation (27 genes), regulation of gene transcription/expression (9 genes), cell differentiation (2 genes), angiogenesis (3 genes), signal transduction (2 genes), apoptosis (2 genes), other (5 genes) or unknown function (5 genes) (FIG. 4).

Of these 55 genes, 47 were found to be present in the Ingenuity Pathway

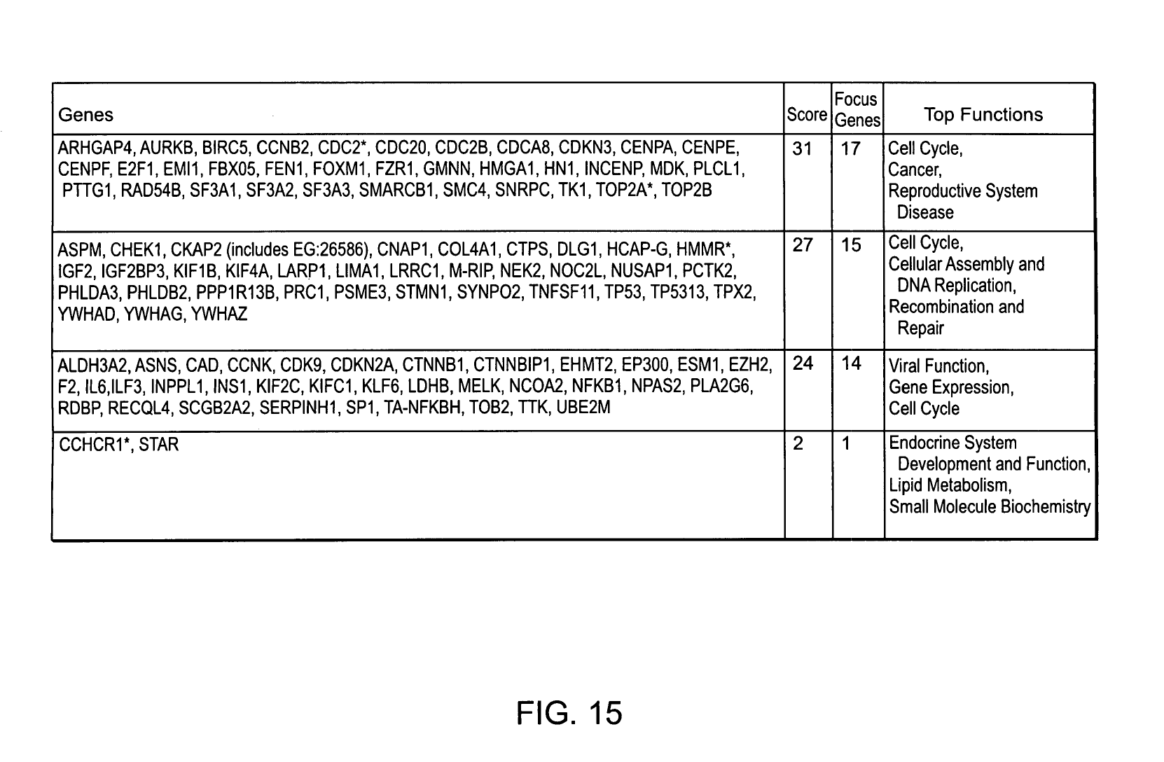

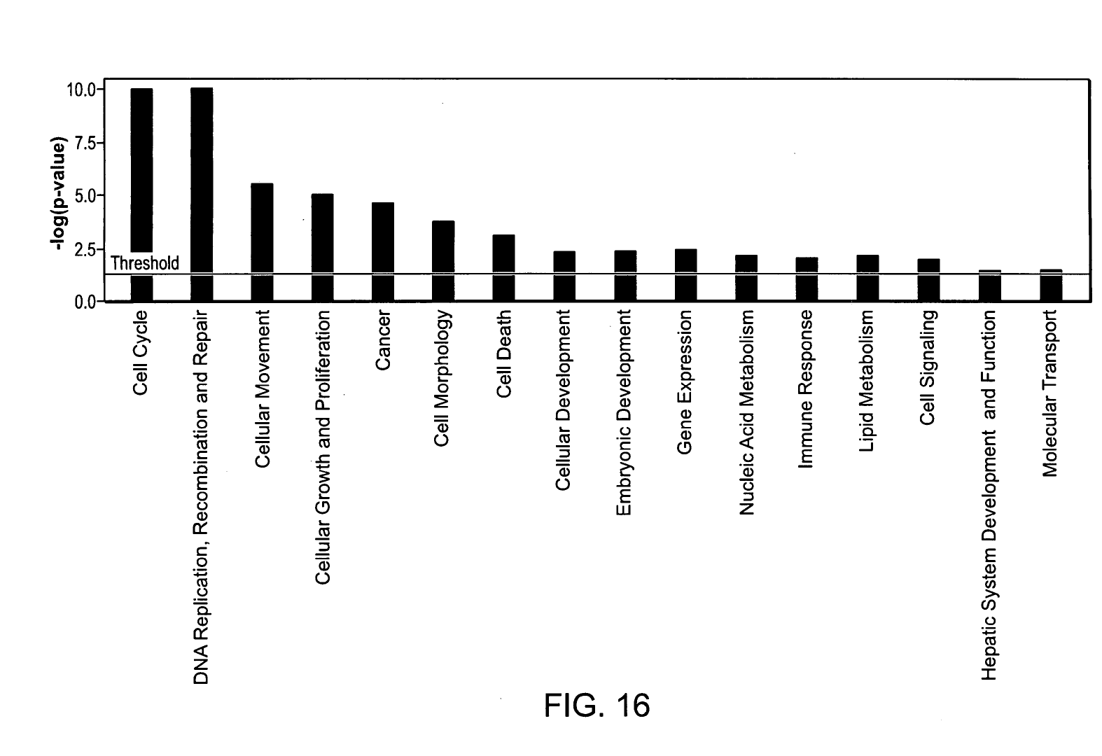

Analysis database, wherein 32 were designated as being involved in the cell cycle, 14 in regulation of gene expression and 1 in lipid metabolism (FIG. 15). Among the 32 genes involved in the cell cycle, 17 were associated with cancer and 15 were associated with DNA replication, repair and/or recombination (FIG. 15). The results of the Ingenuity analysis revealed that the 47 differentially-expressed genes in the database were highly enriched for genes associated with cell cycle and DNA replication/repair functions (p values at 10⁻¹⁰ using right-tailed Fisher's exact test), as well as for cell movement, cellular growth and cancer (FIG. 16).

The 16 probe-sets that showed specific expression in non-tumorous, normal liver tissue were determined to include genes having a variety of functions, including functions related to immune responses (3 genes), sugar binding (2 genes), drug metabolism (2 genes), binding of corticotropin releasing hormone (1 gene), muscle contraction/digestion (1 gene), carbohydrate metabolism (1 gene), lipid/cholesterol metabolism (1 gene), potassium ion transport (1 gene), scavenger receptor activity (1 gene), cell motility (1 gene), cell cycle (1 gene), and cell adhesion (1 gene) (FIG. 5).

### Example 3

### Genes Displaying Significant Differential Expression can Differentiate Neoplastic and Normal Tissues

### Materials and Methods

Hierarchical clustering analyses were performed as described in Example 1.

### Results

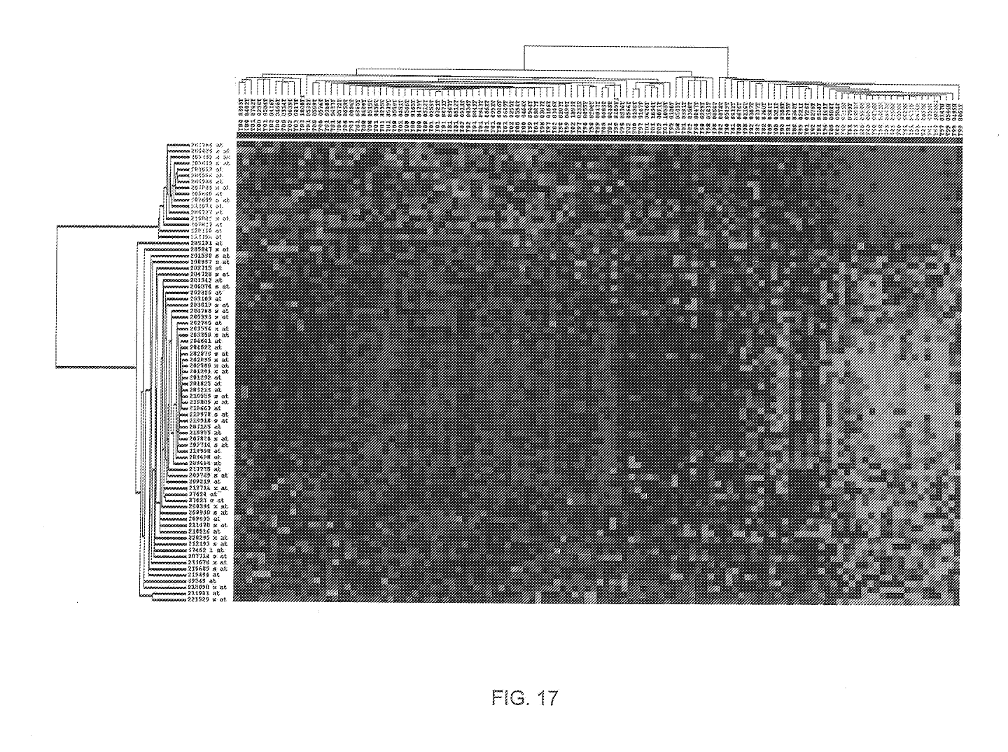

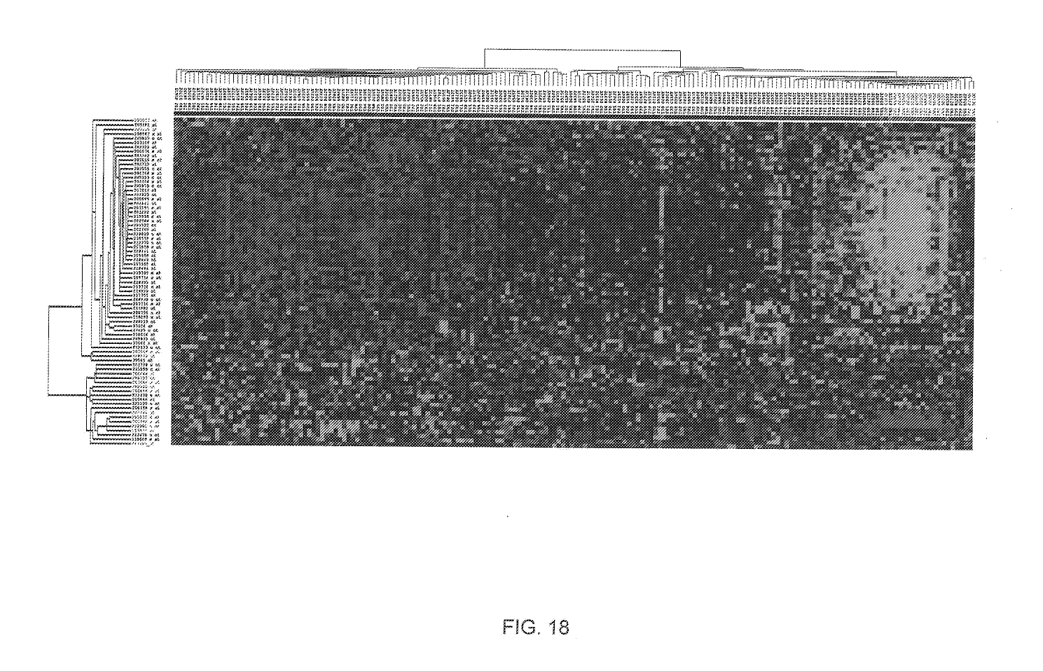

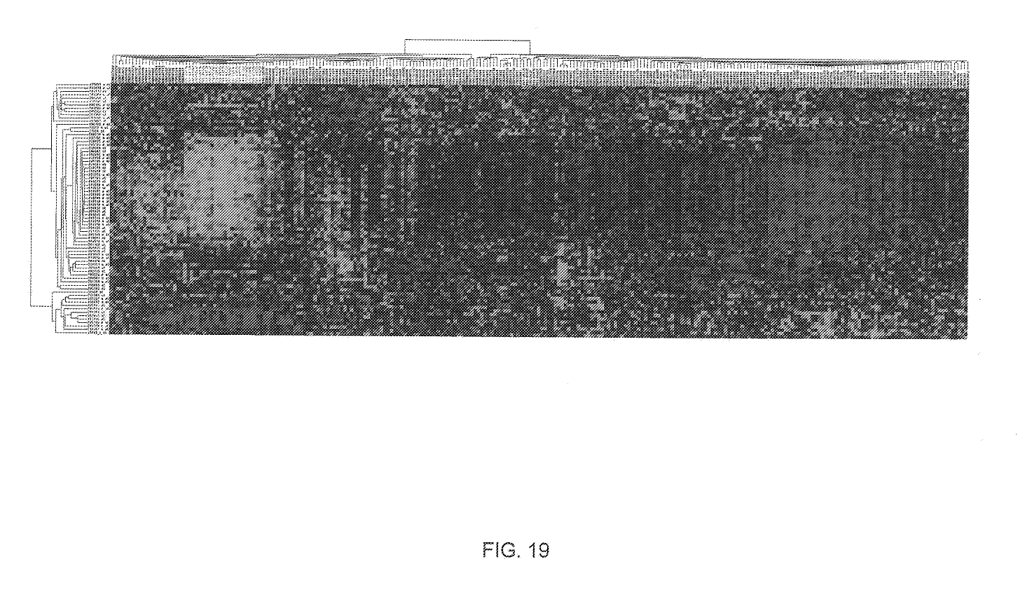

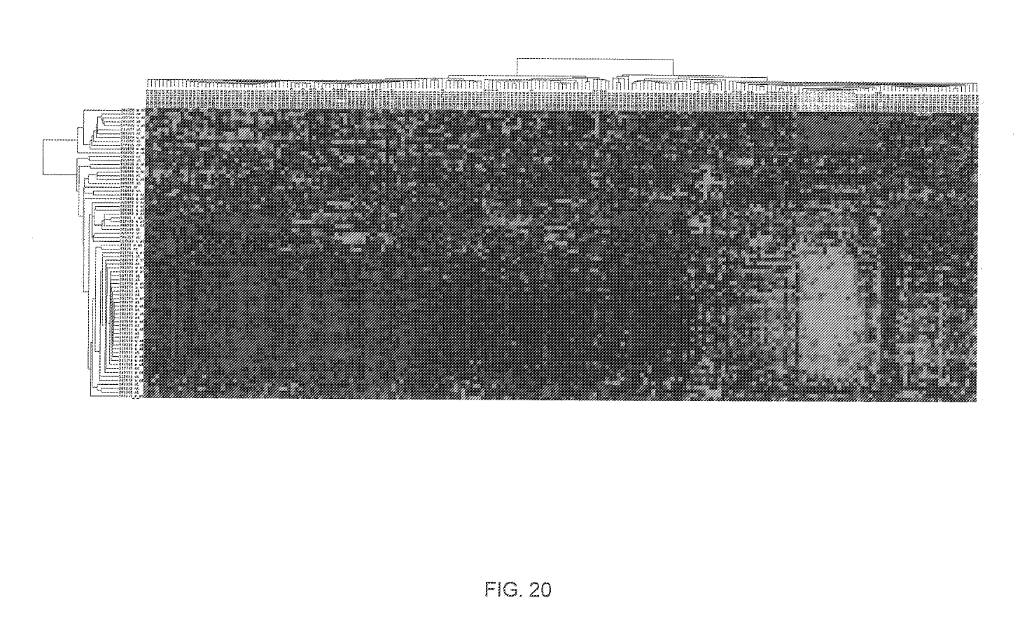

The majority of genes (55) represented by the 75 probe-sets identified in Example 1 were tumor-specific and were identified as being involved in the cell cycle and/or cell proliferation (FIGS. 4, 5 and 15), both of which are hallmarks of a neoplasm. To determine whether these 75 probe-sets are able to differentiate different types of cancers from normal tissues, hierarchical clustering analyses were performed on gene expression profiling data from six different types of major cancers, which included hepatocellular carcinoma, nasopharyngeal cancer, breast cancer, lung cancer, renal cell carcinoma, and colon cancer, and their corresponding normal tissues. The results showed that the 75 probe-sets readily differentiated neoplastic tissues from corresponding non-neoplastic normal tissues for all six types of cancers evaluated in this study (FIGS. 17-22).

To confirm this finding, statistical comparisons of gene expression in cancer and normal tissues were conducted for each of the 75 probe-sets using the datasets in the SCIANTIS™ System Pro database for the twenty different subtypes of cancer chosen for this study. Specifically, a two-sample Welch's t-test was performed for each gene for all 20 types of cancer. Hierarchical clustering analysis was then conducted using the t-values obtained from these comparisons (FIGS. 23A,B). High positive t-values were calculated for all tumor-specific probe-sets, while negative t-values were calculated for all normal tissue-specific probe-sets.

For any given cancer, a large number of genes showing significant differential expression between tumor and normal tissues is expected. Consistent with this expectation, 52% of probe-sets (n=22,283) in the dataset showed statistically significant (i.e., p-values <0.05) differences in gene expression between infiltrating ductal carcinomas and normal breast tissues. Thus, random selection of any group of genes is likely to include some genes that are differentially expressed between tumor and normal tissues. Therefore, it is critical to ensure that probe sets identified as differentially expressed between paired HCC and adjacent non-tumorous tissue samples are significantly greater in number than any randomly selected 75 probe-sets.

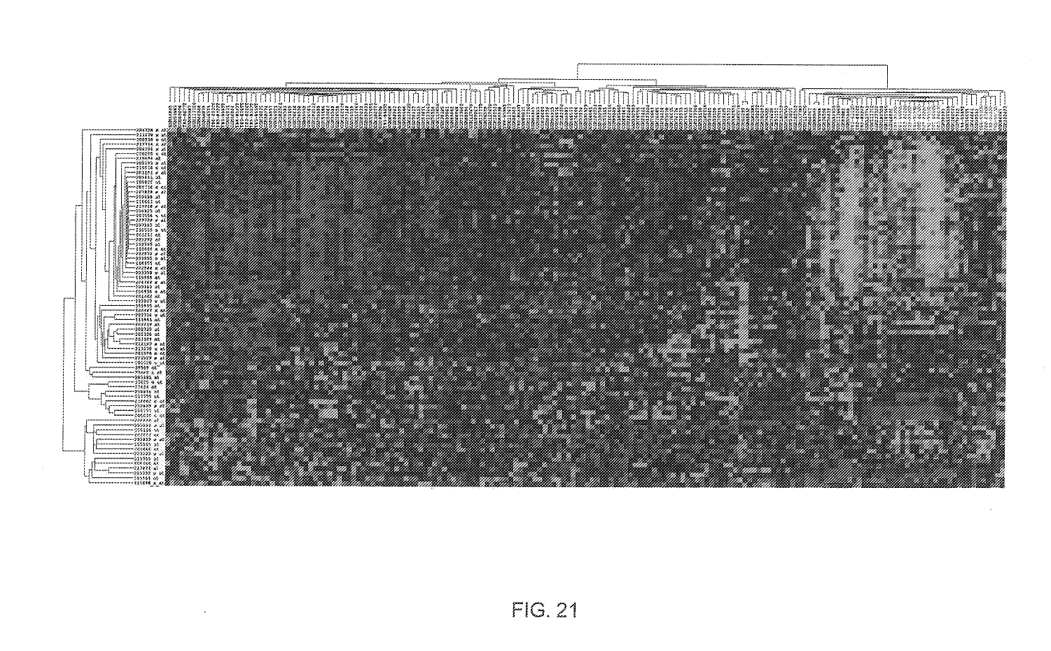

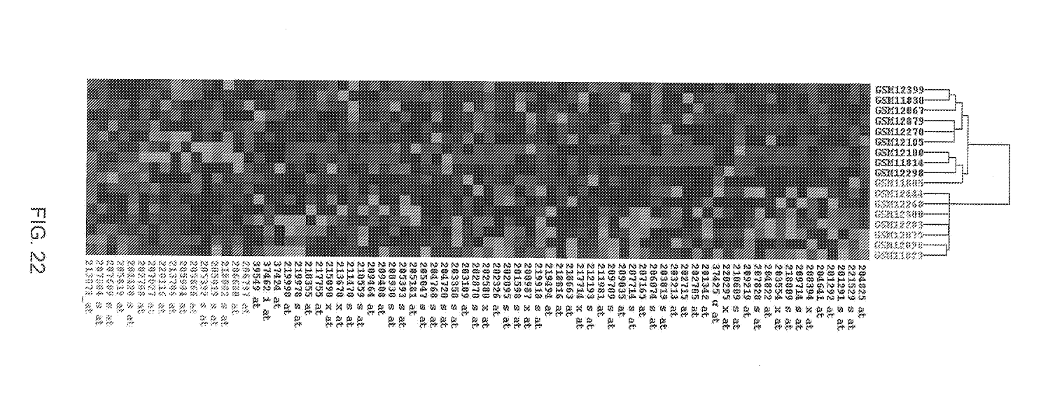

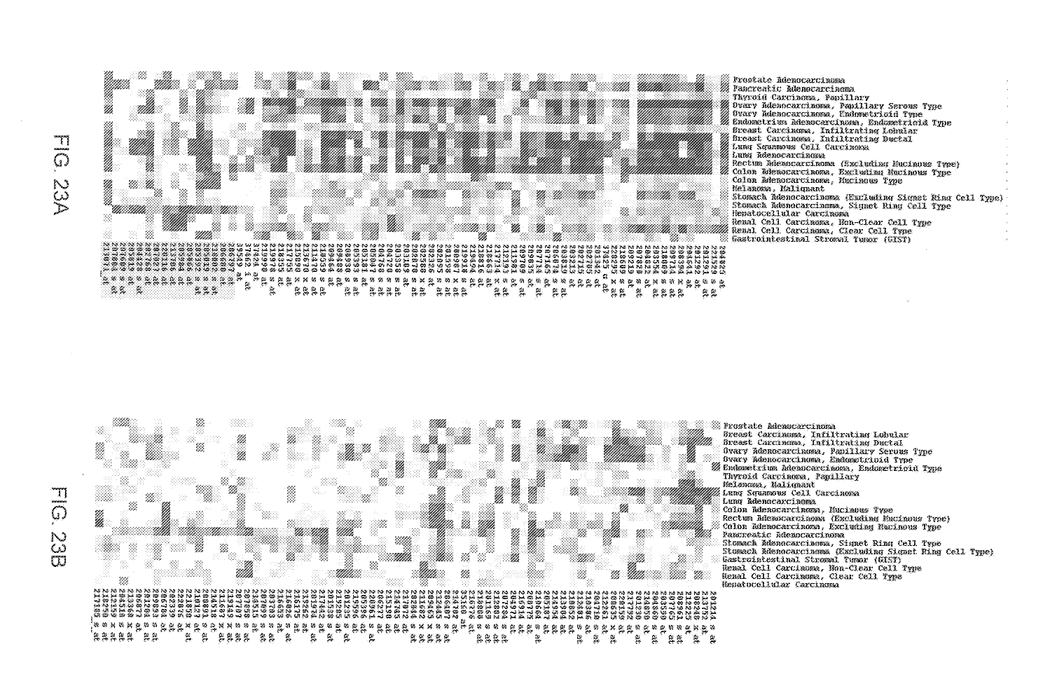

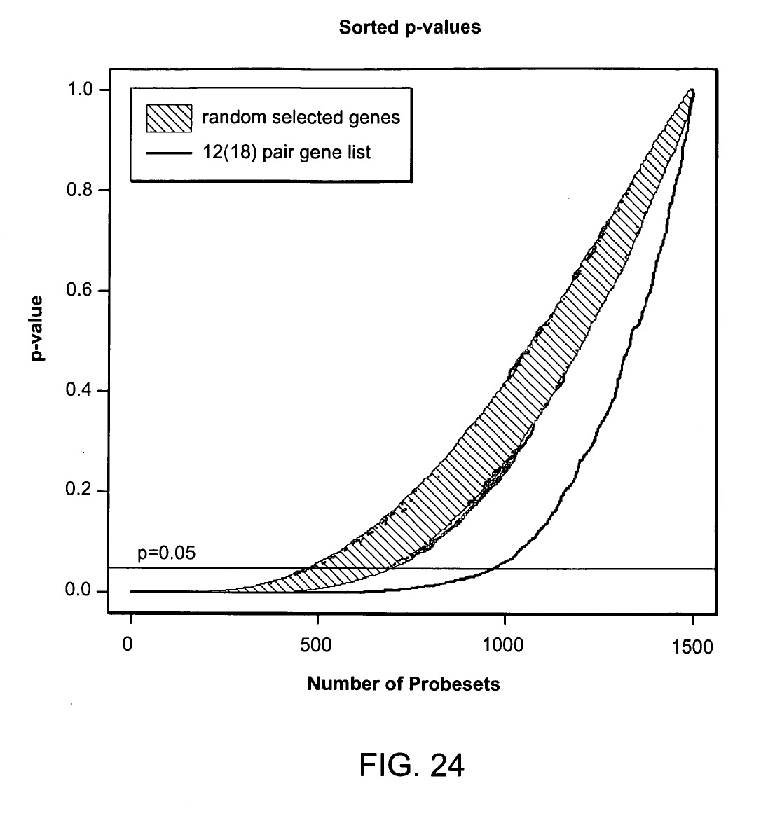

Accordingly, a control study was performed in which seventy-five (75) probe-sets were randomly selected 10,000 times. Gene expression intensities in cancer and normal tissues were compared for each gene represented in the randomly selected probe-sets using the SCIANTIS™ gene expression datasets for the 20 different subtypes of cancer and corresponding normal tissues selected for this study, as described in Example 1. The results demonstrated that genes represented by the 75 probe-sets identified in our study as being differentially expressed between HCC and corresponding normal tissues significantly outnumber the number of randomly selected 75 probe-sets that were differentially expressed between HCC and corresponding normal tissues (FIG. 24).

These results support the conclusion that the genes represented by the 75 probe-sets identified in this study (see Example 1) constitute a common neoplastic signature (CNS), and that expression of these genes and their products (e.g., proteins, peptides, mRNA) can be used as universal markers for cancer.

### Example 4

### Correlation of Expression of 75 Probe-Sets with Cellular Proliferation

### Materials and Methods

### Hierarchical Clustering

Hierarchical clustering analyses were performed as described in Example 1.

### Statistical Analyses

Statistical analyses, including Chi-square test, Fisher's exact test, t-test, and survival analyses (log-rank and Wilcoxon tests), were conducted using SAS software (Version 9.1.3). To assess how the expression of each tumor-specific gene in the common neoplastic signature was correlated with time-dependent overall or distant metastasis-free survival, Cox regression analysis based on proportional hazards model was performed using S-plus software (Version 6) for the datasets of HCC, NPC or breast cancer.

### Results

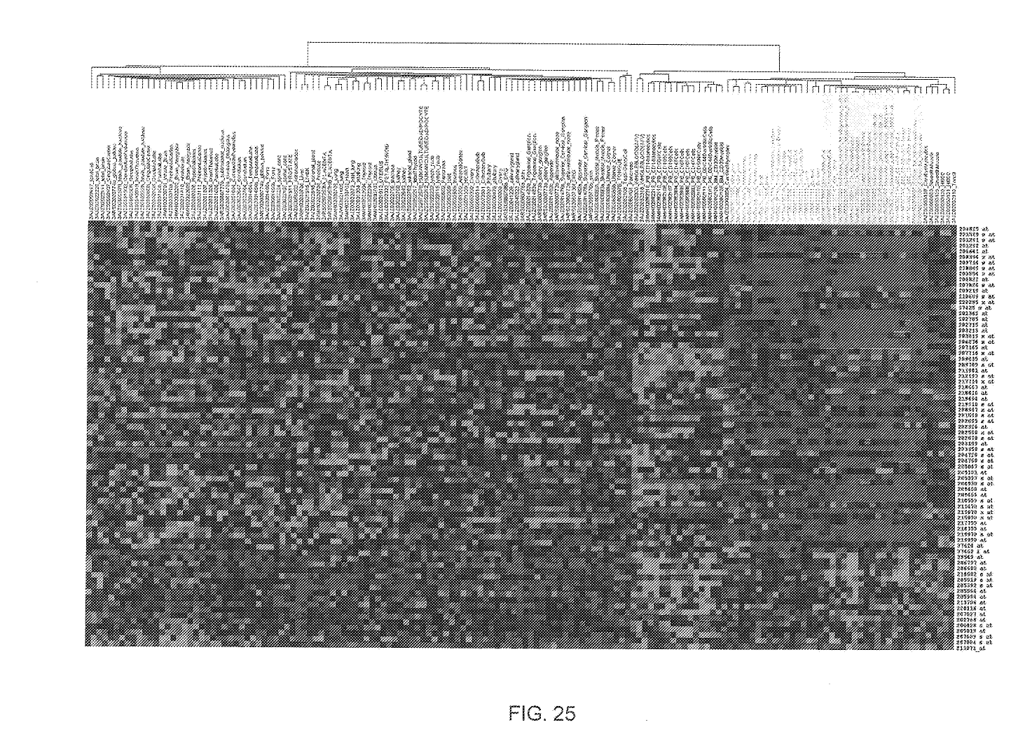

If expression of the genes in the common neoplastic signature is associated with cellular proliferation, hierarchical cluster analysis should reveal elevated expression of these genes in different types of normal tissues and organs that have high proliferation activities. The heat map of hierarchical clustering analysis revealed that genes represented by the 59 tumor-specific probe-sets had elevated expression in highly proliferative normal tissues and organs including bone marrow (hematopoietic organ), thymus, uterus and testis (FIG. 25). Organs and tissues from central nervous system known to be proliferatively quiescent showed significantly reduced expression of most of the tumor-specific probe-sets (FIG. 25).

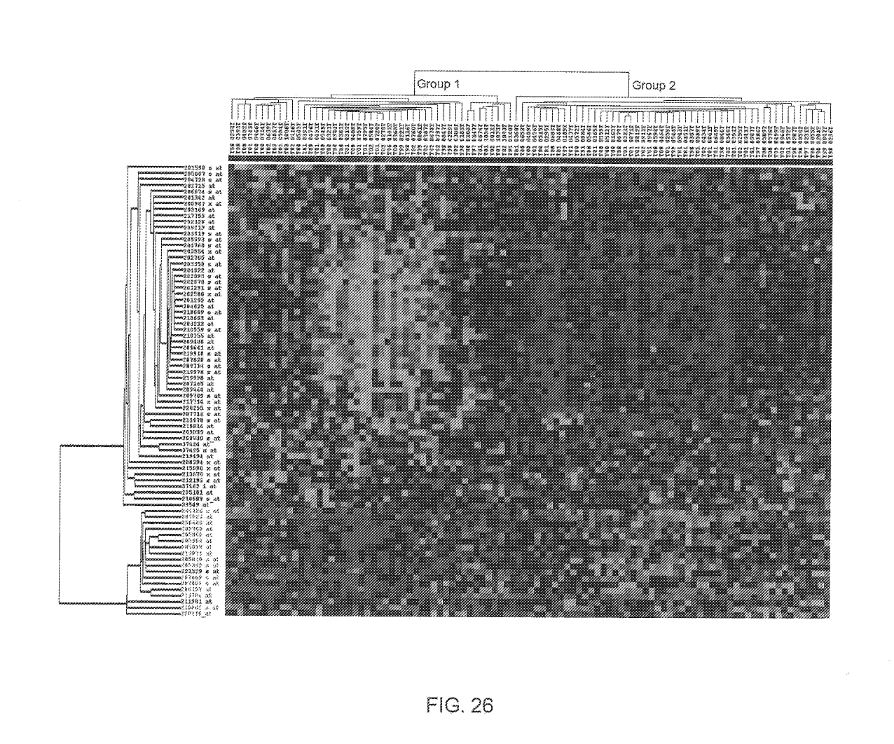

Based on these results, it was hypothesized that cancers with much higher expression of the 59 tumor-specific probe-sets genes would be more proliferative and correlate with larger tumor size and/or a more advanced TNM stage of patients. To test this hypothesis, hierarchical cluster analyses were conducted on breast cancer (n=295), HCC (n=100) and nasopharyngeal carcinomas (n=260), because data regarding tumor size and TNM stage were available for these types of cancer. Each type of cancer was classified into two groups according to gene expression of the 75 probe-sets (FIGS. 26-28). One group had high expression, and the other group had lower expression, of the 55 tumor-specific probe-sets genes (FIGS. 26-28). The two groups of each type of cancer were then correlated with tumor sizes or TNM stages. The results showed that increased expression of the 59 tumor-specific probe-sets correlated with massive HCC tumors (diameter of a tumor ≧10 cm versus nodular types of ≦10 cm) (p=0.009), larger breast cancer tumors (diameter>2 cm versus≦2 cm) (p=0.0005) and more advanced TNM stage of nasopharyngeal carcinoma (stages III+IV versus stages I+II) (p=0.027) (Table 7). All these findings support the conclusion that expression of the 59 tumor-specific probe-sets in the common neoplastic signature reflects the cell proliferation activity of both neoplastic and normal tissues.

TABLE 7
Correlation of hierarchical clusters of HCC, NPC and breast cancer
with different clinical parameters by Fisher's exact test.

| | |
|---|---|
| Clinical Variate | P-values |

Hepatocellular Carcinoma (n = 100)

| | |
|---|---|
| Differentiation Grade (I vs. II vs. III) | 0.0069 |
| Tumor size (≧10 cm vs <10 cm) | 0.0093 |
| Death | 0.0297 |

Nasopharyngeal Carcinoma (n = 168)

| | |
|---|---|
| Distant Metastasis | 0.00098 |
| Stage (1 vs. 2 vs. 3 vs. 4) | 0.1075 |
| Death | 0.1244 |

Breast Cancer (n = 295)

| | |
|---|---|
| Differentiation Grade (I vs. II vs. III) | <.0001 |
| Tumor size (≦2 cm vs >2 cm) | 0.0005 |
| Death | <.0001 |

### Example 5

### Expression of Common Neoplastic Signature Genes Correlates with Survival

### Materials and Methods

### Hierarchical Clustering

Hierarchical clustering analyses were performed as described in Example 1.

### Statistical Analyses

Statistical analyses were performed as described in Example 4.

### Results

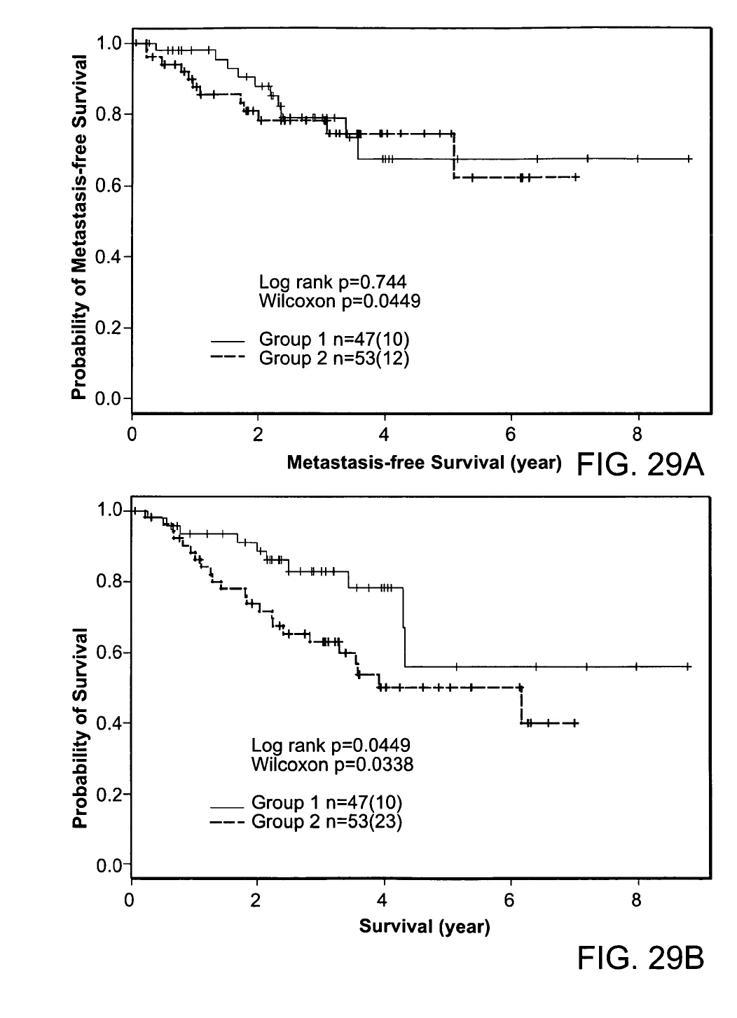

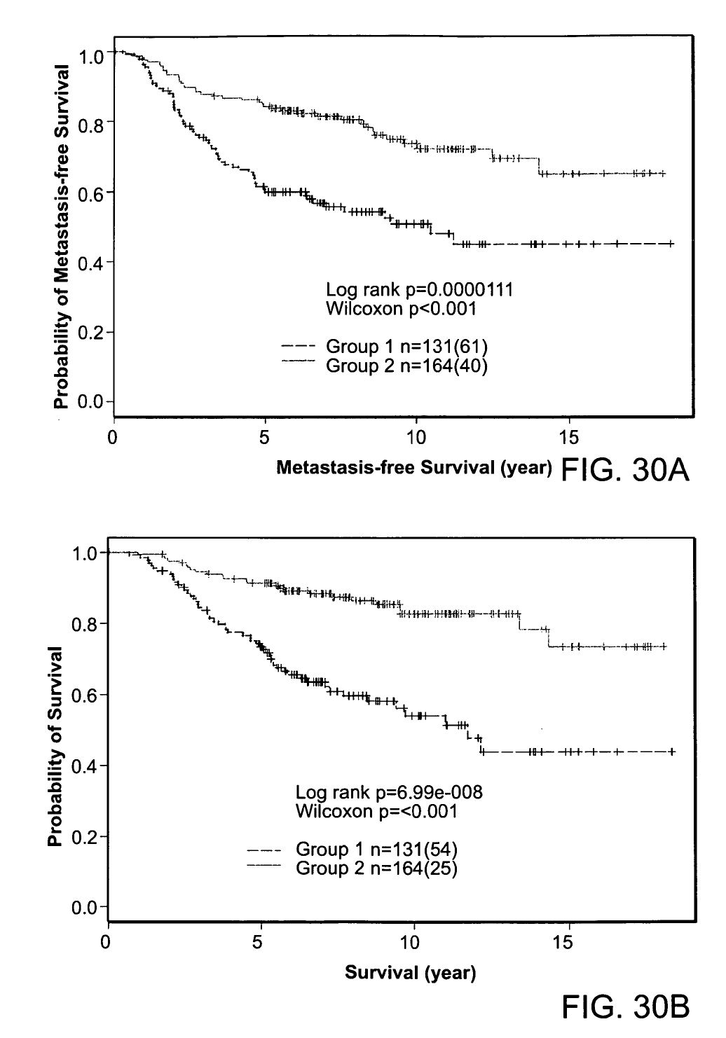

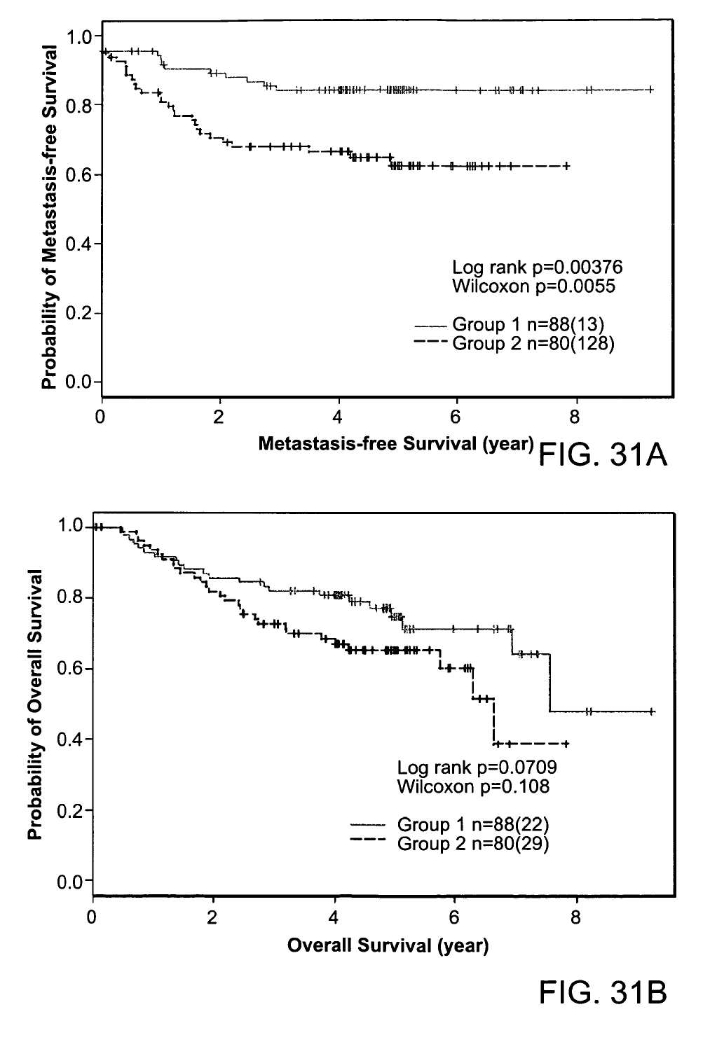

To determine whether tumors displaying increased expression of the 55 genes represented by the 59 tumor-specific probe-sets, and reduced expression of the 16 genes represented by the 16 normal tissue-specific probe-sets, are associated with a poor survival outcome relative to other tumors, the same HCC, breast cancer and nasopharyngeal carcinoma samples described in Example 4 were classified by hierarchical clustering analysis (FIGS. 26-28) with respect to distant-metastasis free survival and overall survival. The results of this analysis showed that HCC and breast cancer patients with increased expression of the 59 tumor-specific probe-sets had significantly reduced overall survival with p-values of 0.037 and 6.9×10⁻⁸, respectively (FIGS. 29 and 30). Nasopharyngeal carcinoma and breast cancer patients with increased expression of the 59 tumor-specific probe-sets exhibited shorter distant metastasis free survival with log-rank test p-values of 0.0038 and 1.1×10⁻⁵, respectively (FIGS. 30 and 31). These results indicate that the 75-probe-set gene signature, and, in particular, the 59 tumor-specific probe-sets, have prognostic value for different subtypes of cancers.

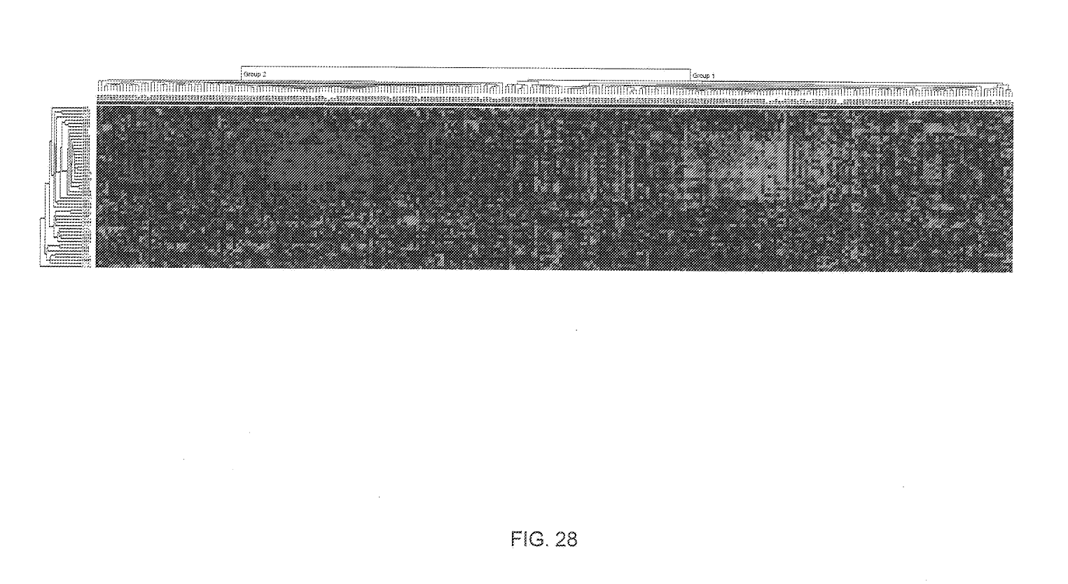

Notably, expression of the genes represented by these 75-probe sets, which were identified by gene expression differences between hepatocellular carcinoma and non-tumorous liver tissues, could be used successfully to classify breast cancers according to survival and risk for distant metastasis (FIGS. 28 and 30) based on a breast cancer dataset generated using a different, non-Affymetrix microarray platform. This cross-platform application further suggests that these genes represent a common neoplastic signature genes with clinical relevance.

### Example 6

### Expression of Common Neoplastic Signature Genes Correlates with Tumor Differentiation

### Materials and Methods

### Hierarchical Clustering

Hierarchical clustering analyses were performed as described in Example 1.

### Statistical Analyses

Statistical analyses were performed as described in Example 4.

### Results

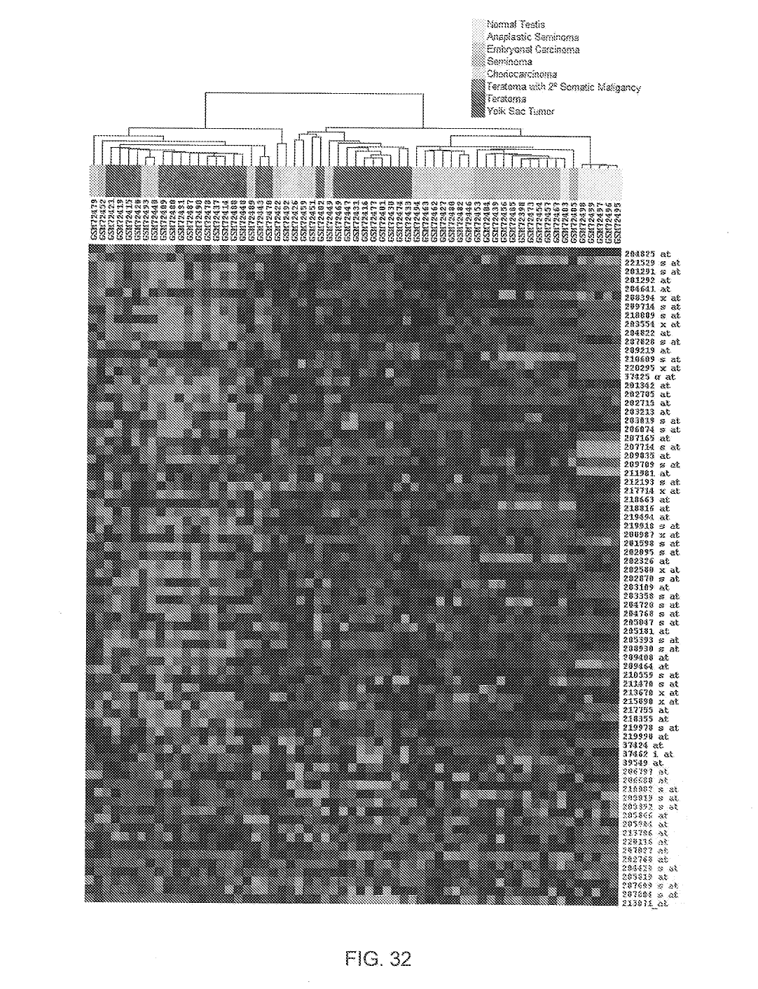

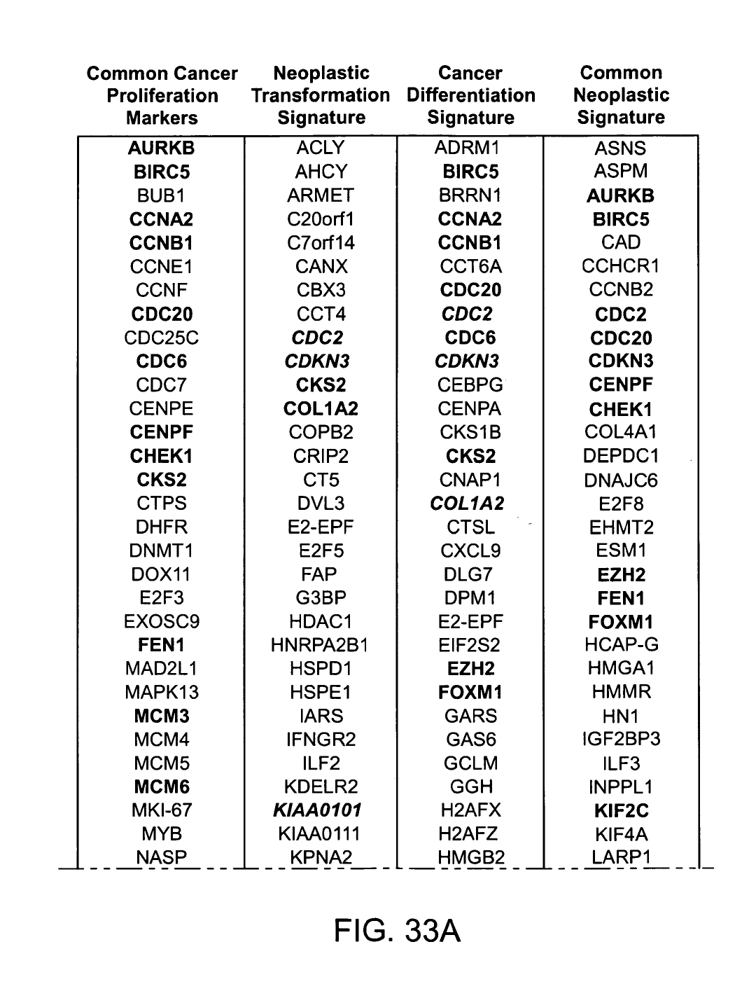

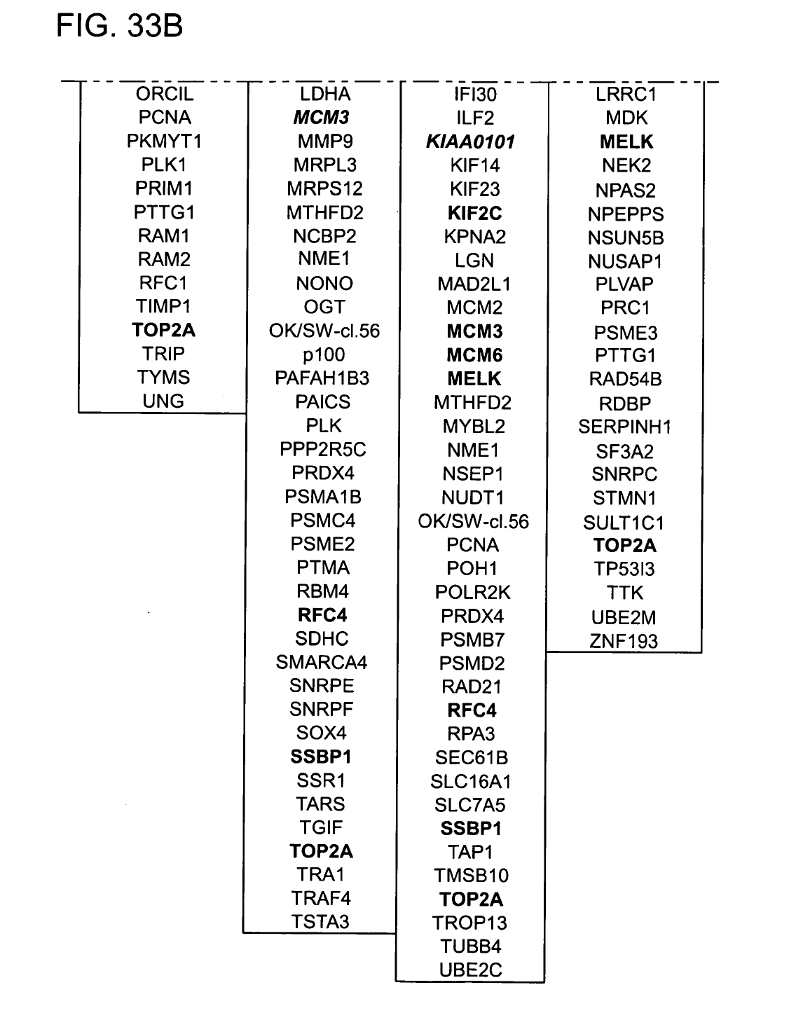

It is well known that tumors having poor clinical outcomes are frequently poorly differentiated. To determine whether increased expression of the 55 genes represented by the 59 tumor-specific probe-sets are associated with poor tumor differentiation, hierarchical clustering analysis was conducted on adult male germ cell tumors with different degrees of differentiation. The results showed that “teratomas” known to contain highly differentiated mature tissues were clustered together with reduced expression of the 59 tumor-specific probe-sets and increased expression of the 16 normal tissue-specific probe-sets (FIG. 32). In contrast, the much less differentiated embryonal carcinoma, yolk sac tumor and seminoma were clustered together with increased expression of the 59 tumor-specific probe-sets and reduced expression of the 16 normal tissue-specific probe-sets (FIG. 32). Normal testis tissue was clustered together with less differentiated germ cell tumors because it contains highly proliferative germ cells.

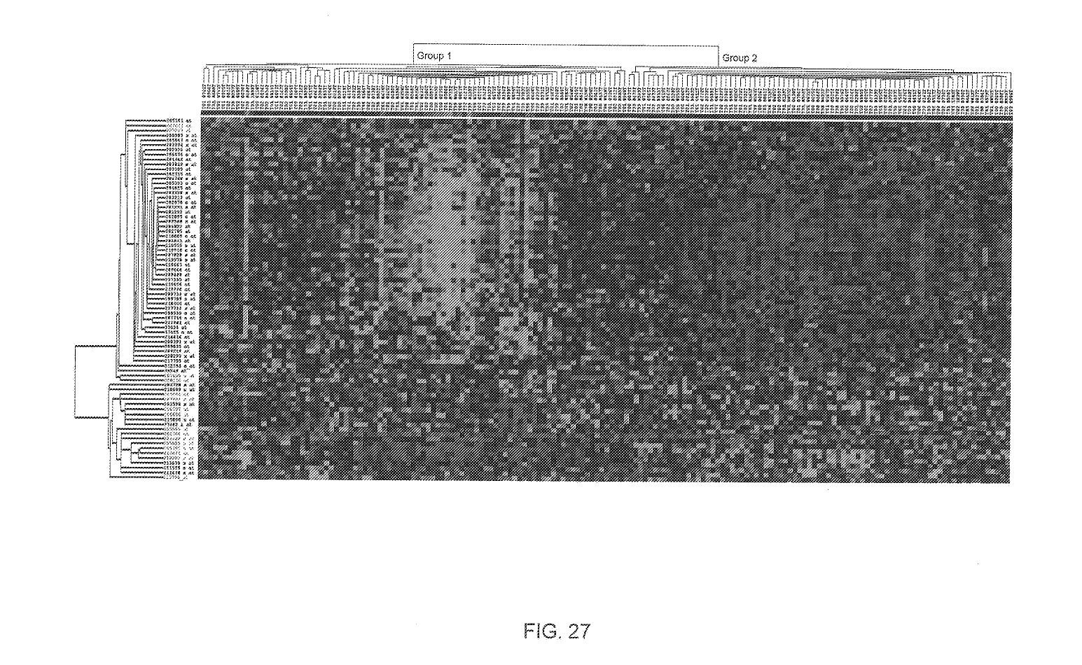

To determine whether differentiation grades of HCC and breast cancer tumors clustered according to the gene expression intensities of the 75 probe-sets identified in Example 1, a statistical correlation study was conducted (FIGS. 26 and 27). These two types of cancer were chosen because tumor differentiation grade data were available. The p-values for correlation between differentiation grades (i.e., well, moderate and poor) and tumor subsets were 0.007 and <0.0001 for HCC and breast cancer, respectively, as determined by hierarchical clustering analysis using the 75 probe-sets (Table 7). These results indicate that increased expression of the 59-tumor-specific probe-sets is associated with reduced tumor differentiation.

### Example 7

### Identification of Genes Associated with Distant Metastasis or Survival

As discussed in Example 5, 55 different genes represented by 59 tumor-specific probe-sets were closely associated with survival and/or distant metastasis in three very different types of cancers (FIGS. 29-31). To identify which of the 55 tumor-specific genes were involved in survival and metastasis for these three types of cancers, the expression intensities of the 55 genes were correlated with time to development of first distant metastasis and time to death of HCC, NPC and breast cancer patients. Genes that showed a significant association (p<0.05) with distant-metastasis free survival or overall survival in each of these three types of cancer are listed in Tables 8A and 8B. Specifically, increased expression of PRC1, CENPF, RDBP, CCNB2 and RAD54B was associated with increased risk of distant metastasis in all three different types of cancers (Table 8A), while increased expression of CDC2, CCHCR1, and HMGA1 were associated with shorter survival in all three different types of cancers (Table 8B). These results suggest that these particular genes play pivotal roles in distant metastasis and/or determination of survival in a variety of different cancers, and could serve as therapeutic targets for control of distant metastasis and/or improvement of survival. Thus, products and functional pathways of the aforementioned genes could also serve as targets for development of new drugs to control cancer growth and metastasis.

TABLE 8A
Genes associated with distant metastasis-free survival
in hepatocellular carcinoma (HCC), nasopharyngeal
carcinoma (NPC) and breast cancer (BRC).

| | Genes Associated with Distant Metastasis | | | | |
|---|---|---|---|---|---|
| Cancer Type | PRC1 | CENPF | RDBP | CCNB2 | RAD54B |
| HCC | + | + | + | + | + |
| NPC | + | + | + | + | + |
| BRC | + | + | + | + | + |

TABLE 8B
Genes associated with overall survival in hepatocellular carcinoma
(HCC), nasopharyngeal carcinoma (NPC) and breast cancer (BRC).

| | Genes Associated with Survival | | |
|---|---|---|---|
| Cancer Type | CDC2 | CCHCR1 | HMGA1 |
| HCC | + | + | + |
| NPC | + | + | + |
| BRC | + | * | * |

HCC: hepatocellular carcinoma (n = 100)
NPC: Nasopharyngeal carcinoma (n = 168)
BRC: Breast cancer (n = 295)
* CCHCR1 and HMGA1 genes were not present in the microarrays used to study BRC.

The relevant teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A method of diagnosing whether a subject has a cancer, comprising detecting the level of expression of a subset of genes in a sample from the subject, wherein the genes in said subset:
a) are selected from the group consisting of MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARP1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1 and NUSAP1; and
b) are overexpressed in the cancer,
wherein increased levels of expression of the genes of the subset in the sample from the subject, relative to a control, indicate that the subject has the cancer.

2. The method of claim 1, wherein the subset consists of at least about twenty genes of said group.

3. The method of claim 1, wherein the cancer is selected from the group consisting of breast cancer, colon cancer, endometrial cancer, renal cell carcinoma, liver cancer, lung cancer, ovarian cancer, pancreatic cancer, prostate cancer, rectal cancer, skin cancer, stomach cancer, and thyroid cancer.

4. The method of claim 1, wherein the cancer is selected from the group consisting of hepatocellular carcinoma, nasopharyngeal cancer, breast cancer, lung cancer, renal cell carcinoma and colon cancer.

5.-12. (canceled)

13. A method of providing a subject that has a cancer with a prognosis for risk of metastasis, comprising:
a) detecting the level of expression of one or more genes selected from the group consisting of PRC1, CENPF, RDBP, CCNB2 and RAD54B in a sample from the subject, and
b) comparing said level of expression to a control,
wherein an increased level of expression of said one or more genes in the sample from the subject, relative to the control, indicates a prognosis for increased risk of metastasis of said cancer.

14. The method of claim 13, wherein the subject has a cancer selected from the group consisting of hepatocellular carcinoma, nasopharyngeal cancer, and breast cancer.

15. The method of claim 13, wherein the risk of metastasis is a risk of distant metastasis.

16.-22. (canceled)

23. A method of providing a survival prognosis for a subject that has a cancer, comprising:
a) detecting the level of expression of one or more genes selected from the group consisting of CDC2, CCHCR1 and HMGA1 in a sample from the subject, and
b) comparing said level of expression to a control,
wherein an increased level of expression of said one or more genes in the sample from the subject, relative to the control, indicates a prognosis for shorter survival.

24. The method of claim 23, wherein the subject has a cancer selected from the group consisting of hepatocellular carcinoma, nasopharyngeal cancer, and breast cancer.

25.-30. (canceled)

31. A kit for diagnosing whether a subject has a cancer, comprising a collection of probes capable of detecting the level of expression of at least about ten genes selected from the group consisting of MELK, PLVAP, TOP2A, NEK2, CDKN3, PRC1, ESM1, PTTG1, TTK, CENPF, RDBP, CCHCR1, DEPDC1, TP5313, CCNB2, CAD, CDC2, HMMR, STMN1, HCAP-G, MDK, RAD54B, ASPM, HMGA1, SNRPC, IGF2BP3, SERPINH1, COL4A1, LARP1, LRRC1, FOXM1, CDC20, UBE2M, DNAJC6, FEN1, ASNS, CHEK1, KIF2C, AURKB, NPEPPS, KIF4A, E2F8, EZH2, ZNF193, ILF3, EHMT2, SF3A2, NPAS2, PSME3, INPPL1, BIRC5, SULT1C1, NSUN5B, HN1 and NUSAP1.

32. The kit of claim 31, wherein the probes comprise nucleic acid probes or antibody probes.

33.-35. (canceled)

36. The kit of claim 31, wherein the collection of probes is capable of detecting the level of expression of all genes in said group.

37. A kit for providing a subject that has a cancer with a prognosis for risk of metastasis of said cancer, comprising a probe that is capable of detecting the level of expression of one or more genes selected from the group consisting of PRC1, CENPF, RDBP, CCNB2 and RAD54B.

38. The kit of claim 37, wherein the probe comprises a nucleic acid probe that specifically hybridizes to an mRNA encoded by said one or more genes, or an antibody probe that specifically binds to a protein encoded by said one or more genes.

39.-40. (canceled)

41. A kit for determining a survival prognosis for a subject that has a cancer, comprising a probe that is capable of detecting the level of expression of one or more genes selected from the group consisting of CDC2, CCHCR1 and HMGA1.

42. The kit of claim 41, wherein the probe comprises a nucleic acid probe that specifically hybridizes to an mRNA encoded by said one or more genes, or an antibody probe that specifically binds to a protein encoded by said one or more genes.

43.-45. (canceled)

46. A method of diagnosing whether a subject has a cancer, comprising detecting the level of expression of a subset of genes in a sample from the subject, wherein the genes in said subset:
a) are selected from the group consisting of NAT2, CD5L, CXCL14, VIPR1, CCL14/15, FCN3, CRHBP, GPD1, KCNN2, HGFAC, FOSB, LCAT, MARCO, CYP1A2, FCN2, and DPT; and
b) are underexpressed in the cancer,
wherein decreased levels of expression of the genes of the subset in the sample from the subject, relative to a control, indicate that the subject has the cancer.

47. The method of claim 46, wherein the cancer is selected from the group consisting of breast cancer, colon cancer, endometrial cancer, renal cell carcinoma, liver cancer, lung cancer, ovarian cancer, pancreatic cancer, prostate cancer, rectal cancer, skin cancer, stomach cancer, and thyroid cancer.

48. The method of claim 46, wherein the cancer is selected from the group consisting of hepatocellular carcinoma, nasopharyngeal cancer, breast cancer, lung cancer, renal cell carcinoma and colon cancer.

49.-56. (canceled)

57. A kit for diagnosing whether a subject has a cancer, comprising a collection of probes capable of detecting the level of expression of at least about five genes selected from the group consisting of NAT2, CD5L, CXCL14, VIPR1, CCL14/15, FCN3, CRHBP, GPD1, KCNN2, HGFAC, FOSB, LCAT, MARCO, CYP1A2, FCN2, and DPT.

58. The kit of claim 57, wherein the probes comprises nucleic acid probes or antibody probes.

59.-61. (canceled)

62. The kit of claim 57, wherein the collection of probes is capable of detecting the level of expression of all genes in said group.
